(19) United States
(12) Patent Application Publication
(10) Pub. No.: US 2014/0249301 A1
(43) Pub. Date: Sep. 4, 2014

(54) METHODS FOR ENABLING FARNESENE ACCUMULATION IN PLANTS AND RELATED COMPOSITIONS

(71) Applicant: Chromatin, Inc., Chicago, IL (US)

(72) Inventor: John C. Steffens, Chapel Hill, NC (US)

(73) Assignee: Chromatin, Inc., Chicago, IL (US)

(21) Appl. No.: 14/191,067

(22) Filed: Feb. 26, 2014

Related U.S. Application Data
(60) Provisional application No. 61/769,196, filed on Feb. 26, 2013.

(51) Int. Cl. C12P 19/44; C12P 7/04; C12P 5/00

(52) U.S. Cl. CPC C12P 19/44; C12P 5/007; C12P 7/04
USPC 536/18.5; 435/167; 435/157; 435/74; 435/419; 800/298; 568/913

(57) ABSTRACT

The invention provides novel methods and compositions directed to farnesol production, accumulation and cellular sequestration in plants. More specifically, the methods of the invention comprise modifying plant cells that express farnesene to convert the farnesene to farnesol, and in some cases, to farnesol glycoside, such as farnesol glucoside. In other embodiments, carbon flux is shunted towards sesquiterpene production by applying certain plant growth regulators and herbicides to increase sesquiterpene production.

### CROSS REFERENCE TO RELATED APPLICATION

This application claims priority to Steffens, J., U.S. Provisional Application No. 61/769,196, “METHODS FOR ENABLING FARNESENE ACCUMULATION IN PLANTS AND RELATED COMPOSITIONS” filed Feb. 26, 2013, incorporated by reference herein in its entirety.

### GOVERNMENT SUPPORT

The subject matter of this application was in part funded by the Department of Energy, the Advanced Research Projects Agency—Energy under the award “Plant Based Sesquiterpene Biofuels,” DE-AR0000208. The government may have certain rights in this invention.

### FIELD OF THE INVENTION

The present invention relates to methods and compositions directed to accumulating terpenoids in plant cells through their hydroxylation and glycosylation in plants, such as in sorghum, including sweet sorghum, sugarcane, guayule, and the like.

### COMPACT DISC FOR SEQUENCE LISTINGS AND TABLES

Not applicable.

### BACKGROUND OF THE INVENTION

All citations are incorporated herein by reference.

Sustainable Energy

Agricultural and aquacultural crops have the potential to meet escalating global demands for affordable and sustainable production of food, fuels, fibers, therapeutics, and biofeedstocks.

Development of sustainable sources of domestic energy is crucial for the US to achieve energy independence. In 2010, the US produced 13.2 billion gallons of ethanol from corn grain and 315 million gallons of biodiesel from soybeans as the predominant forms of liquid biofuels (Board, 2011; RFA, 2011). It is expected that biofuels based on corn grain and soybeans will not exceed 15.8 billion gallons in the long term. Although efforts to convert biomass to biofuel by either enzymatic or thermochemical processes will continue to contribute towards energy independence (Lin and Tanaka, 2006; Nigam and Singh, 2011), this process alone is not enough to achieve the target goals of biofuel production. It is projected that only 12% of all liquid fuels produced in the US can be derived from renewable sources by 2035, far below the mandated 30% (Newell, 2011). To reach the target levels of 30% of all liquid fuels consumed in US by 2035, new and innovative biofuel production methodologies must be employed.

Terpenoids

Because of their abundance and high energy content, terpenoids provide an attractive alternative to current biofuels (Bohlmann and Keeling, 2008; Pourbafrani et al., 2010; Wu et al., 2006). The terpenoid biosynthetic pathway is ubiquitous in plants and produces over 40,000 structures, forming the largest class of plant metabolites (Bohlmann and Keeling, 2008). Research on terpenoids has focused primarily on uses as flavor components or scent compounds (Cheng et al., 2007a). Terpene-based biofuel production has focused on the use of micro-organisms, including yeast and bacterial systems, to generate poly-terpenoid fuels (Fischer et al., 2008; Nigam and Singh, 2011; Peralta-Yahya and Keasling, 2010). However, it is unclear whether this microorganism-based approach will allow production of isoprenoid resins at sufficient quantities, or sufficiently low cost, to supplement and/or replace liquid fossil fuel consumption. Further, this process is energy-intensive, requiring a supply of plant-based sugars for large scale fermentation, constant maintenance of temperature and nutrition to micro-organism cultures, and the development of immense infrastructure to support meaningful, large-scale micro-organism growth. Attempts have been made to overcome these obstacles by engineering the production of biodiesel hydrocarbons in algal systems and thus defray some of the energy cost by harnessing the photosynthetic capacity of these organisms. Algal systems still require significant inputs of energy to maintain temperature and salt equilibria and have so far not produced biodiesel in sufficient quantities to offset the costs of building the large-scale bio-reactors necessary for algal biodiesel production.

Sorghum and Guayule

Sorghum, as well as other carbon-reservoir-plants, such as sugarcane and guayule, have been shown to be amenable to genetic engineering to increase terpenoid production, including farnesene (Blakeslee et al., 2013).

Guayule, a dicotyledonous desert shrub native to the Southwestern US and Mexico thrives in semi-arid desert environments and marginal lands not currently used for food production (Bonner, 1943; Hammond, 1965; Tipton and Gregg, 1982). Guayule has long been established as a source of natural rubber, resins, and bioactive terpenoid compounds. In addition to producing hydrocarbon rubber polymers during the winter (Cornish and Backhaus, 2003), guayule produces and stores a high-energy hydrocarbon terpenoid resin in specialized resin vessels throughout the year (Coffelt et al., 2009). Further, guayule can be grown with greatly reduced inputs of water (Dierig et al., 2001) and pesticides (compared to traditional crops such as nuts, alfalfa, and cotton), and on lands in the Southwestern US not currently utilized for food production (Whitworth, 1991).

Guayule has been successfully transformed to express several genes involved in the synthesis of terpenoid precursors; mono-, sesqui- and di-terpenoid molecules; and isoprenoid rubber polymers using Agrobacterium-mediated transformation (Veatch et al., 2005). Further, methods have been developed for the optimal extraction of resin and terpenoid moieties from harvested guayule tissues (Pearson et al., 2010; Salvucci et al., 2009). Finally, transgenic guayule lines have been successfully brought to field trials, where they have been demonstrated to accumulate increased accumulations of terpenoid-rich resins (Veatch et al., 2005).

Sorghum, a C4 monocotyledonous grass grown in the southwestern, central and Midwestern US, has high photosynthetic efficiency, water and nutrient efficiency, stress tolerance, and is unmatched in its diversity of germplasm including starch (grain) types, high sugar (sweet) types, and high-biomass photoperiod sensitive (forage) types. Sorghum outperforms corn in regions with low annual rainfall, making it an ideal crop for the semi-arid regions (Zhan et al., 2003). Sorghum is suited to acreage where corn, soybean and cotton grow.

In both guayule and sorghum, as in many other plants, terpenoid synthesis (FIG. 1) occurs through the cytosolic mevalonic acid pathway (MVA) and the methylerythritol phosphate pathway (MEP), the latter of which is localized to the plastidic compartment (Cheng et al., 2007a).

Plants that accumulate β-farnesene-rich terpene resins are useful in that the β-farnesene can be converted to liquid fuels. Such crops yield liquid fuel requiring little external processing (Connor and Atsumi, 2010).

Even though engineering sorghum, sugarcane and guayule to accumulate β-farnesene is now possible, prior attempts to achieve sesquiterpene accumulation in plants show that, for compounds less volatile than farnesene, about 50% of the sesquiterpene synthesized is lost via volatilization under greenhouse conditions. Furthermore, adverse phenotypes are also known to accompany modifications to cause sesquiterpene accumulation. One source of adverse phenotypes may arise from partitioning of free farnesene into membrane bilayers, altering fluidity and function.

### SUMMARY OF THE INVENTION

In a first aspect, the invention is directed to methods of accumulating at least one sesquiterpene in a plant cell comprising a) expressing a transgene encoding an exogenous polypeptide that hydroxylates at least one sesquiterpene in the plant cell, and b) accumulating the hydroxylated sesquiterpene within the plant cell, wherein the hydroxylated sesquiterpene is less volatile than at least one unhydroxylated sesquiterpene and thereby accumulates within the plant cells. In such aspect, the plant cell may produce a greater amount of the at least one sesquiterpene when compared to that produced by a non-transgenic cell of the same genotype that does not express the transgene. The plant cell may also be a transgenic plant cell engineered to produce elevated amounts of the at least one sesquiterpene when compared to the amount of at least one sesquiterpene produced by a non-transgenic cell of the same genotype in the absence of expression of the exogenous polypeptide. In some cases, the at least one sesquiterpene is farnesene, and the hydroxylated sesquiterpene is farnesol. In such aspect, the exogenous polypeptide can be a farnesene synthase having a carbocation reaction intermediate quenchable by water (a farnesol synthase) or a cytochrome P450 enzyme. The farnesol synthase can comprise an amino acid sequence having at least 70%-100%, including 70%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity to SEQ ID NO:2 or SEQ ID NO:10, or an active fragment thereof. In some case, the farnesene synthase is encoded by the nucleic acid sequence of SEQ ID NO:1 or SEQ ID NO:9. The plant cell may be from any plant, such as sorghum (especially sweet sorghum), sugar cane, and guayule.

In a second aspect, the invention is directed to methods of accumulating farnesol in a plant cell comprising a) expressing a transgene encoding an exogenous farnesyl diphosphate synthase polypeptide, and b) accumulating the farnesol within the plant cell. The exogenous farnesyl diphosphate synthase polypeptide can comprise an amino acid sequence having at least 70%-100%, including 70%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity to SEQ ID NO:4, or an active fragment thereof. In some embodiments, the exogenous farnesyl diphosphate synthase polypeptide is encoded by a polynucleotide of SEQ ID NO:3. The plant cell may be from any plant, such as sorghum (especially sweet sorghum), sugar cane, and guayule.

In a third aspect, the invention is directed to methods of accumulating farnesol glycoside in a plant cell comprising a) expressing a first transgene encoding an exogenous farnesyl diphosphate synthase polypeptide to accumulate farnesol, b) expressing a second transgene encoding an exogenous glycosyl transferase that modifies the farnesol to a farnesol glycoside, and c) accumulating the farnesol glycoside within the plant cell, wherein the farnesol glycoside is less volatile than farnesol. In some aspects, the exogenous glycosyl transferase is a UDPG:glucosyl transferase polypeptide. In yet other aspects, the UDPG:glucosyl transferase polypeptide can comprise an amino acid sequence having at least 70%-100%, including 70%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity to SEQ ID NOs:6, 12, 14, 16, 18, 20, 22, or 24, or an active fragment thereof. In yet other aspects, the UDPG:glucosyl transferase polypeptide is encoded by the nucleic acid sequence of SEQ ID NOs:5, 11, 13, 15, 17, 19, 21, or 23. In further aspects, a third transgene is expressed that encodes an exogenous linalool synthase. Such linalool synthase can comprise an amino acid sequence having at least 70%-100%, including 70%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity to SEQ ID NO:8, or an active fragment thereof. In some aspects, the exogenous linalool synthase polypeptide is encoded by a nucleic acid sequence of SEQ ID NO:7. The plant cell may be from any plant, such as sorghum (especially sweet sorghum), sugar cane, and guayule.

In yet a fourth aspect, the invention is directed to methods of protecting a plant cell from phytotoxicity of at least one hydroxylated sesquiterpene in a plant cell comprising a) expressing a transgene encoding an exogenous glycosyl transferase that modifies the at least one hydroxylated sesquiterpene to a hydroxylated sesquiterpene glycoside in the plant cell, and b) the hydroxylated sesquiterpene glycoside has a less phytotoxic effect on the plant cell than the at least one hydroxylated sesquiterpene. In some aspects, the hydroxylated sesquiterpene glycoside is less volatile than a non-glycoside hydroxylated sesquiterpene. The at least one sesquiterpene can be farnesene, and the at least one hydroxylated sesquiterpene can be farnesol. In further aspects, the methods comprise expressing an exogenous glycosyl transferase which may comprise expressing a UDPG:glucosyl transferase. Such UDPG:glucosyl transferase polypeptide can comprise an amino acid sequence having at least 70%-100%, including 70%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity to SEQ ID NOs:6, 12, 14, 16, 18, 20, 22, or 24, or an active fragment thereof. In some aspects, the UDPG:glucosyl transferase polypeptide is encoded by a nucleic acid sequence of SEQ ID NOs:5, 11, 13, 15, 17, 19, 21, or 23. The methods can further comprise expressing a second transgene encoding an exogenous linalool synthase polypeptide, wherein the exogenous linalool synthase polypeptide can comprise an amino acid sequence having at least 70%-100%, including 70%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity to SEQ ID NO:8, or an active fragment thereof. In some aspects, the exogenous linalool synthase polypeptide is encoded by a nucleic acid sequence of SEQ ID NO:7. In some aspects, the method comprises transgenic plant cells that produce a greater amount of the at least one sesquiterpene when compared to that produced by a non-transgenic cell of the same genotype that does not express the transgene(s). The plant cell may be from any plant, such as sorghum (especially sweet sorghum), sugar cane, and guayule.

In a fifth aspect, the invention is directed to methods of sequestering at least one sesquiterpene in vacuole of a plant cell comprising a) expressing a transgene encoding an exogenous glycosyl transferase polypeptide that modifies the at least one sesquiterpene to a sesquiterpene glycoside in the plant cell, and b) accumulating the sesquiterpene glycoside in a vacuole of the plant cell and thereby sequestering the sesquiterpene glycoside in the plant cell. In such aspects, the exogenous glycosyl transferase polypeptide can be a UDPG:glucosyl transferase. In yet other aspects, the UDPG:glucosyl transferase can comprise an amino acid sequence having at least 0%-100%, including 70%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity with SEQ ID NOS:6, 12, 14, 16, 18, 20, 22, or 24. In yet other aspects, the UDPG:glucosyl transferase is encoded by a nucleic acid sequence of SEQ ID NOs:5, 11, 13, 15, 17, 19, 21, or 23. In further aspects, the methods further comprise expressing a second transgene encoding an exogenous linalool synthase polypeptide. Such exogenous linalool synthase polypeptide can comprise an amino acid sequence having at least 70%-100%, including 70%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity to SEQ ID NO:8, or an active fragment thereof. In some aspects, the exogenous linalool synthase polypeptide is encoded by a nucleic acid sequence of SEQ ID NO:7. The plant cell may be from any plant, such as sorghum (especially sweet sorghum), sugar cane, and guayule.

In a sixth aspect, the invention is directed to transgenic plant cells comprising at least one transgene encoding for at least one selected from the group consisting of an exogenous: farnesol synthase, farnesyl diphosphate synthase polypeptide, a cytochrome P450 enzyme, a glycosyl transferase polypeptide, and a linalool synthase polypeptide. When the at least one transgene comprises exogenous farnesol synthase, the farnesol synthase can comprise an amino acid sequence having at least 70%-100%, including 70%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity to SEQ ID NO:2 or SEQ ID NO:10, or an active fragment thereof. In some aspects, the exogenous farnesol synthase is encoded by a nucleic acid sequence of SEQ ID NO:1 or SEQ ID NO:9. When the at least one transgene comprises exogenous farnesyl diphosphate synthase, the farnesyl diphosphate synthase can comprise an amino acid sequence having at least 70%-100%, including 70%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity to SEQ ID NO:4, or an active fragment thereof. In some aspects, the exogenous farnesyl diphosphate synthase is encoded by a polynucleotide of SEQ ID NO:3. When the transgene comprise an exogenous glycosyl transferase, such glycosyl transferase can be UDPG:glucosyl transferase. In some aspects, the exogenous UDPG:glucosyl transferase can comprise an amino acid sequence having at least 70%-100%, including 70%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity to SEQ ID NOs:6, 12, 14, 16, 18, 20, 22, or 24, or an active fragment thereof. In some aspects, the exogenous UDPG:glucosyl transferase is encoded by a nucleic acid sequence of SEQ ID NOs:5, 11, 13, 15, 17, 19, 21, or 23. When the transgene comprises linalool synthase, such linalool synthase can comprise an amino acid sequence having at least 70%-100%, including 70%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity to SEQ ID NO:8, or an active fragment thereof. In some aspects, the exogenous linalool synthase is encoded by a nucleic acid sequence of SEQ ID NO:7. In yet further aspects, the transgenic cell comprises at least two, three, four, five, six, seven, eight, nine, ten or more transgenes. In yet further aspects, the transgenic plant cell produces a greater amount of the at least one sesquiterpene when compared to that produced by a non-transgenic cell of the same genotype that does not express the transgene. In additional aspects, the invention is directed to transgenic plant tissue comprising the transgenic plant cells of the invention, transgenic plants, and transgenic plant parts. The transgenic plant cell may be from any plant, such as sorghum (especially sweet sorghum), sugar cane, and guayule.

In a seventh aspect, the invention is directed to methods of harvesting farnesol from a transgenic plant cell comprising chopping or grinding plant tissue comprising transgenic cells that have accumulated farnesol. Such methods can comprise harvesting farnesol from any previously described transgenic cell set forth in the sixth aspect.

In an eighth aspect, the invention is directed to methods of harvesting a farnesol glycoside from a transgenic plant cell comprising chopping or grinding plant tissue comprising transgenic cells that have accumulated a farnesol glycoside. The method can include harvesting from plant tissue comprising transgenic cells comprising a transgene that comprise an exogenous glycosyl transferase, such glycosyl transferase can be UDPG:glucosyl transferase. In some aspects, the exogenous UDPG:glucosyl transferase can comprise an amino acid sequence having at least 70%-100%, including 70%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity to SEQ ID NOs:6, 12, 14, 16, 18, 20, 22, or 24, or an active fragment thereof. In some aspects, the exogenous UDPG:glucosyl transferase is encoded by a nucleic acid sequence of SEQ ID NOs:5, 11, 13, 15, 17, 19, 21, or 23. The transgenic cell may further comprise an exogenous linalool synthase, such linalool synthase can comprise an amino acid sequence having at least 70%-100%, including 70%, 80%, 81%, 82%, 83%, 84%, 85%, 86%, 87%, 88%, 89%, 90%, 91%, 92%, 93%, 94%, 95%, 96%, 97%, 98%, 99%, and 100% sequence identity to SEQ ID NO:8, or an active fragment thereof. In some aspects, the exogenous linalool synthase is encoded by a nucleic acid sequence of SEQ ID NO:7. In such methods, endogenous glycosidases convert the farnesol glycoside to farnesol; in additional aspects, at least one exogenous glycosidase is added to the chopped or ground plant tissue, such as before, during or after chopping or grinding. The plant cells may be from any plant, such as sorghum (especially sweet sorghum), sugar cane, and guayule.

In a ninth aspect, the invention is directed to methods of channeling carbon flux in a plant cell comprising a) down-regulating carotenoid or sterol biosynthetic branch point enzymes, and b) channeling carbon flux towards sesquiterpene production. The plant cell may be from any plant, such as sorghum (especially sweet sorghum), sugar cane, and guayule. In some aspects, the plant cell is a transgenic plant cell the produces a greater amount of at least one sesquiterpene when compared to a non-transgenic plant cell of the same genotype. In yet other aspects, channeling carbon flux comprises down-regulating carotenoid and sterol biosynthetic branch point enzymes. Such down-regulation can comprise introducing into the plant cell at least one construct comprising a transgene; and which down-regulation is constitutive or conditional. The methods can further comprise contacting the plant cell with a plant growth regulator or an herbicide. Examples of plant growth regulators are ethylene stimulators, phytoene synthase inhibitors, squalene epoxidase inhibitors of sterol biosynthesis, and compounds stimulating systemic acquired resistance. Examples of ethylene stimulators are ethephon, etacelasil, glyoxime, and 1-aminocyclopropane-I-carboxylic acid. Examples of phytoene synthase inhibitors are gerfelin or a bisphosphonate analog of geranyl pyrophosphate. Examples of squalene epoxidase inhibitors of sterol biosynthesis are monooxygenase inhibitor-based herbicides in the allylamine and thiocarbamate classes, including pyributicarb.

### BRIEF DESCRIPTION OF SEVERAL VIEWS OF THE DRAWINGS

FIG. 1 shows a schema of β-farnesene production strategies. Glycolysis breaks sucrose into pyruvate which is processed into the terpenoid precursors dimethylallyl pyrophosphate/isopentenyl pyrophosphate (DMAPP/IPP) via the mevalonic acid (MVA) (cytosol) or methylerythritol phosphate (MEP) (chloroplast) pathway. IPP subunits are assembled into farnesyl-pyrophosphate (FPP), which is then converted into β-farnesene. Proteins catalyzing rate-limiting steps are 3-hydroxy-3-methyl-glutaryl-CoA reductase (HMG-CoA reductase), FPP synthase, β-farnesene synthase, and 1-deoxy-D-xylulose-5-phosphate synthase.

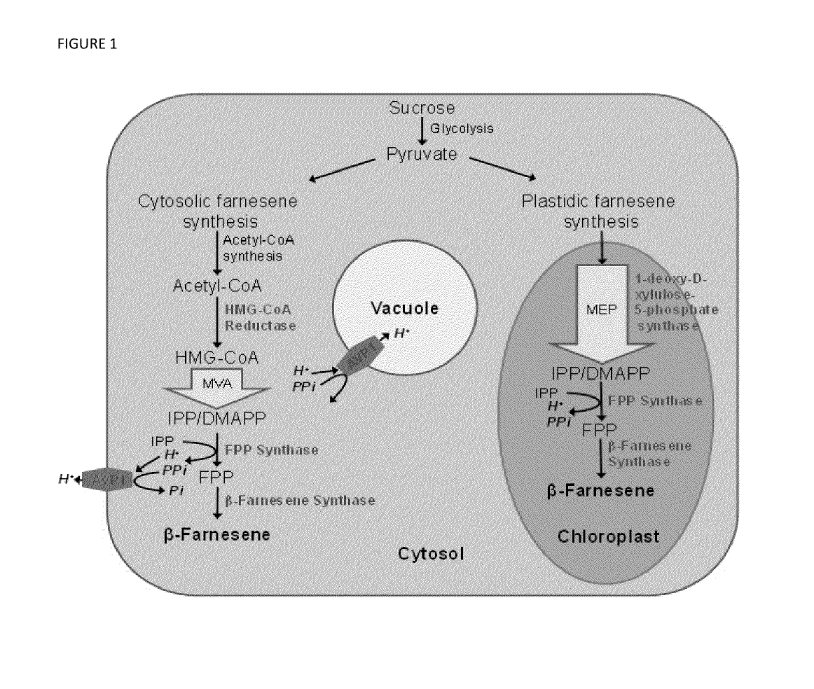

FIG. 2 shows a schematic of a plasmid, “Construct 1,” that can be used in the methods of the invention (in conjunction with “Construct 2”). Construct 1 bears rate-limiting genes HMG-CoA reductase and FPP synthase in the production of β-farnesene. Hb-HMGR,

FIG. 3 shows a schematic of a plasmid, “Construct 2,” that can be used in the methods of the invention (also can be in conjunction with “Construct 1”). Construct 2 bears a farnesol synthase (Os (Oryza sativa)-TPS13) and a glycosyl transferase (At (Arabidopsis thaliana)-UGT85A1). Zm-Ubi, Zea mays ubiquitin; OsActin, Oryza sativa actin. See Example 1 for further details.

### DETAILED DESCRIPTION OF THE INVENTION

I. Introduction

The present invention solves the problems of volatilization of target sesquiterpenes and minimizes phenotypic effects from the over-production of sesquiterpenes. The present invention provides methods for enabling accumulation of farnesene at high levels that include: (1) reducing volatilization losses by producing a less-volatile hydroxylated derivative, such as farnesol; (2) further reduction of volatility and reduction of phytotoxicity by conversion of farnesol to a farnesol glycoside; (3) further reduction of toxicity by accumulation of farnesol glycoside in vacuoles; (4) further, conferring the ability to circumvent feedback regulation and accumulate to high levels by sequestration in vacuoles; and (5) liberation of free farnesol upon disruption of plant tissue by means such as chopping, grinding, etc., that result in mixing of vacuolar compartments with endogenous non-specific cytosolic glycosidases.

The present invention further provides for methods that circumvent the default regulation of plant terpenoid pathways (synthesis of carotenoids and sterols at the expense of sesquiterpenes) by conditional, tissue-specific, or constitutive genetic down-regulation of carotenoid and/or sterol biosynthetic branch point enzymes, which can also include treating plants with plant growth regulators or herbicides possessing specific modes of action that lead to inhibition of carotenoid or sterol biosynthesis (or both) and allow channeling of carbon flux toward sesquiterpenes instead of into higher-order terpenes.

In some embodiments of the invention, a guayule, sugarcane or sorghum (such as sweet sorghum) cell is modified to produce large quantities of terpenoids, such as farnesene, is further modified according to the methods of the invention.

In one embodiment, a plant cell, such as a sorghum, sugar cane, or guayule cell, is modified to produce a hydroxylated derivative of farnesene, such as farnesol. In some embodiments, the plant cell is modified to express a farnesene synthase having a carbocation intermediate that is quenchable by water. The farnesene synthase comprises a farnesol synthase activity (“farnesene synthase having farnesol synthase activity” and “farnesol synthase” are used interchangeably herein). In yet other embodiments, the plant cell is modified to express a transgene that results in accumulating farnesyl pyrophosphate (FPP). These transgenic cells accumulate farnesol in greater quantities than a cell of the same genotype not carrying the transgene(s).

In some embodiments, the farnesol produced by the modified cell is converted to a farnesol glycoside; in some of these embodiments, a UDPG:glucosyl transferase transgene is expressed to convert the farnesol to a farnesol glucoside. In other embodiments, farnesol is produced by modifying a plant cell to express a linalool synthase transgene. In some such embodiments, the farnesol glycoside accumulates in plant cell vacuoles. In yet other embodiments, the farnesol glycoside is harvested by grinding or chopping plant tissues comprising modified plant cells to synthesize farnesol glycoside, releasing endogenous glycosidases that convert the farnesol glycoside to farnesol. In yet other such embodiments, an exogenous glycosidase is added, either before grinding and chopping, during grinding and chopping, after grinding and chopping or some combination of timing for adding the glycosidase.

In other embodiments, carbon flux in a plant cell is channeled to sesquiterpenoid production by down-regulating carotenoid or sterol biosynthetic branch point enzymes, or both. Such down-regulation is achieved by introducing one or more transgenes, or contacting the plant cell with a plant growth regulator or an herbicide that has such an effect, or both. Such channeling results in increased production of sesquiterpenes.

Some farnesene synthases are known to follow a reaction mechanism that results in a carbocation reaction intermediate being quenched by water, resulting in formation of farnesol rather than the olefinic farnesene. Alternatively, engineering the accumulation of FPP can lead to accumulation of farnesol via phosphatase or pyrophosphatase action on FPP. Another embodiment includes engineering of cytochrome P450 or other hydroxylating enzyme activities directed against farnesene to lead to production of farnesol. Farnesol is much less volatile than farnesene, and higher levels of sesquiterpene accumulation can thereby be achieved by targeting accumulation of this product rather than farnesene. Moreover there may be higher fuel value inherent in the oxygenated form of farnesene.

Cellular compartmentalization of farnesol brings the advantages of sequestering large quantities of farnesol and removing feedback inhibition of farnesol production. For example, in some embodiments, engineering farnesol accumulation, in conjunction with expression of a farnesol-specific UDPG:glucosyl transferase, results in accumulating farnesol glucoside. The default cellular pathway for glycosides is to be transported across the tonoplast and accumulate in vacuoles. In addition to rendering the product nonvolatile, formation of the glycoside and its removal to the vacuole removes the ability of the compound to participate in the feedback inhibition of cytosolic terpene pathway enzymes, as well as removes its ability to diffuse freely in the cytoplasm. Thus a higher level of sesquiterpene accumulation is achieved by vacuolar or extracellular targeting such as is afforded by glycosylation, and any phytotoxic effects of the compound may also be minimized by its extracellular sequestration.

Synthesis of farnesene derivatives may also require modified crop processing relative to that anticipated for farnesene alone. When cells are disrupted sufficiently to mix vacuolar and cytoplasmic compartments, endogenous non-specific glycosidases can be sufficient to convert farnesol glucoside to farnesol. However, catalytic hydrogenation processing steps, such as that previously suggested for the final step of converting farnesene to C15 alkane, can be sufficient also to simultaneously deglucosylate farnesol glycoside.

To favor the flow of carbon toward sesquiterpenes and away from carotenoids and sterol synthesis, a range of plant growth regulators (PGRs) and herbicides can be applied at various stages of crop development, prior to cutting, or prior to final harvest. For example ethylene-based PGRs (ethephon, etacelasil, glyoxime, 1-aminocyclopropane-1-carboxylic acid (ACC) or other stimulators of ethylene synthesis or production), Geranylgeranyl pyrophosphate (GGPP) inhibitors acting as inhibitors of phytoene synthase (such as the natural product gerfelin or bisphosphonate analogs of geranyl pyrophosphate (GPP), squalene epoxidase inhibitors of sterol biosynthesis (e.g., monooxygenase inhibitor-based herbicides in the allylamine and thiocarbamate classes (such as pyributicarb)), or compounds stimulating systemic acquired resistance (e.g., benzo (1,2,3) thiadiazole-7-carbothioic acid S-methyl ester (also known as acibenzolar-5-methyl, BION®), salicylic acid) are examples of PGRs, herbicides or other chemical classes which can be used to channel carbon flow away from “housekeeping” terpenoids such as carotenoids and sterols and towards sesquiterpenes. Moreover, the application of herbicidal compounds, in cases in which the herbicide ultimately leads to crop death, has additional utility in coordinating the accumulation of sesquiterpenes with crop harvest, or effecting more rapid or consistent dry-down of the crop.

II. Waking and Using the Invention

(Note: definitions are found at the end of the Detailed Description, before the Examples; a Table of Selected Abbreviations is found at the end of the Examples)

The methods of the invention are applicable to any plant or plant cell that produces sesquiterpenoids that includes farnesene. In some cases, a plant or plant cell is modified or engineered to produce farnesene or increased levels of farnesene, through mutation, genetic engineering, or selection of such plants or plant cells. In some embodiments, the plant or the plant cell is further engineered to produce higher amounts of farnesene than when compared to non-modified plants or plant cells of the same genotype.

First, farnesol production in a plant cell is addressed; secondly, a discussion of useful types of vectors for transgenic approaches of the invention, and thirdly, the introduction of such engineered vectors into cells. Fourth, regeneration of transgenic plants is addressed, and then analysis of the transgenic plants. Finally, non-transgenic approaches are discussed for some embodiments of the invention.

Forming Farnesol in Plant Cells

Farnesol is much less volatile than farnesene, and higher levels of sesquiterpene accumulation can thereby be achieved by accumulation this product rather than farnesene. Moreover a higher fuel value inherent in the oxygenated form of farnesene may be realized.

In one embodiment, a plant cell or a plant is modified to have a farnesene synthase which reaction mechanism results in a carbocation reaction intermediate being quenched by water, resulting in farnesol formation. In some embodiments, the plant cell is engineered to produce elevated levels of sesquiterpenes, such as farnesene. Any farnesene synthase that has a reaction mechanism that results in the carbocation reaction intermediate being quenched by water can be used in the methods of the invention (“farnesol synthase”), such as rice farnesol synthase (OsTPS13; SEQ ID NOs:1, 2 (Tables 1 and 2)) (Cheng et al., 2007b) and maize farnesol synthase (SEQ ID NOs:9, 10 (Tables 1 and 2)). Alternatively, engineering the accumulation of FPP can lead to accumulation of farnesol via phosphatase or pyrophosphatase action on FPP; such as expressing a farnesyl diphosphate synthase such as encoded by, for example, ispA from E. coli (SEQ ID NOs:3, 4 (Tables 1 and 2)) (Wang et al.). Another embodiment includes engineering of cytochrome P450 or other hydroxylating enzyme activities directed against farnesene to lead to production of farnesol.

Engineering of farnesol accumulation, in conjunction with expression of a farnesol-specific UDPG:glucosyl transferase (such as UGT72E1 (genomic polynucleotide sequence is shown in Table 1; SEQ ID NOs:5, 6 (and Table 2)) (Lanot et al., 2008), UGT88A1 (SEQ ID NOs:11, 12 (Tables 1 and 2)), UTG85A4 (SEQ ID NOs:13, 14 (Tables 1 and 2)), UTG85A2 (SEQ ID NOs:15, 16 (Tables 1 and 2)), UTG85A1 (SEQ ID NOs:17, 18 (Tables 1 and 2)), UTG85A7 (SEQ ID NOs:19, 20 (Tables 1 and 2)), UTG73C6 (SEQ ID NOs:21, 22 (Tables 1 and 2)), and UTG73C5 (SEQ ID NOs:23, 24) (Tables 1 and 2)) or other genes that increase the concentration of glycosylated products (such as linalool synthase (Aharoni et al., 2003) or S-linalool synthase (Lucker et al., 2001) exemplified as Arabidopsis thaliana linalool synthase in Tables 1 and 2, result in accumulation of farnesol glycosides. The default cellular path for glycosides is for their transport across the tonoplast and accumulation in vacuoles. In addition to rendering the product nonvolatile, formation of the glycoside and its removal to the vacuole removes ability of the compound to participate in the feedback inhibition of cytosolic terpene pathway enzymes, as well as removes its ability to diffuse freely in the cytoplasm. Thus a higher level of sesquiterpene accumulation can be achieved by vacuolar or extracellular targeting such as is afforded by glycosylation (Lim, 2005; Pulido et al., 2012; Wang et al., 2010), and any phytotoxic effects of the compound can be minimized by its cellular sequestration.

TABLE 1
Exemplary polynucleotides

Farnesol synthase (OsTPS13) (Oryza sativa) (SEQ ID NO: 1)

| | |
|---|---|
| atggcgccgg ctttccaccc tgcgatattc ggtgacttct tcatcaacaa cgtccaacca | 60 |
| tcacccaaag agtcagatga atggatggaa gagagagtag atcagttggt tgaggaggta | 120 |
| ggtagaatgc ttgaggtttg caaggatgat gtggtaaagc aaatgaacct ggtggatgtg | 180 |
| ctccaacgtt tgggaataga tcatcatttt gaggagcaga tcgacaccat tctaaaaaat | 240 |
| attcatagag ctgagttcaa tagctctgac ctttacgagg ttgcccttcg gtttcgctta | 300 |
| cttaggaaac aagggtattg ggtctcgcca gatgaattca acaaattcaa agctgaagat | 360 |
| gggagcttta gtagtgatga cataactaat gatccgaagg gcttgttaag tttatacaac | 420 |
| gcagctcacc ttctaactca caacgagaaa gcacttgaag aagctatatt gtttgcaagg | 480 |
| catcacctac aattattgag aggcaacctc gcgtacccat tggatgaaca agtaacacgt | 540 |
| gcccttgaga taccgttacc aaggaccatg aagagagtag aggtgctaaa ttatatcttc | 600 |
| gagtatagcg ctgaagagaa aatgttcaat ccctctattt tggagctagc cgtgcttgat | 660 |
| ttcaatattc tacaaaaagt tcaccaaaat gaactcaagg aaatttgtca gtggtgggag | 720 |
| aatctttcaa gtgacattag actcgactac gtccgtgaac gtgtggttga gtgctatttt | 780 |
| tgtgcatacg ctgcctacta tgaaaaagag cacgcacggg cccgtatgat attcgccaag | 840 |
| aggtgtatgc tattttcact gctcgatgat acatacgatg tgcgtgcaac tttagaggag | 900 |
| gctcgcaagt ttaacgatgc tctgcaaaga tgggacaaga gtgacgtttc acttctacca | 960 |
| gaggacctga agagattttt tctgagcata ataagtaact ttagggagtt tgaggatgaa | 1020 |
| ttggaaccac atgagaaata tcgtaattct tacaacatta aagcgtttca aatactatcg | 1080 |
| agcaatttcc tccaagaagc agaatggttt catcaaaact atattccatg ctttactgat | 1140 |
| catgtgactg tttccctcca gaccggaggt gcaatagagt tacctgttag tttaattgtt | 1200 |
| ggcatgggtg atatagcaac taaggaggtg ttggattggg ccttggctaa ccctgatgct | 1260 |
| ggtagggcct ttgcagaggt ggcacgattc atggatgact tagctgcatc acacagtggg | 1320 |
| agggacaaga tggatgtggc gagcactgtg gagtgttaca tgaacgagca cggggtgacg | 1380 |
| agagaggtcg ccgaggcgaa gatcgctgga atggctgagg acgggtggaa aagcatgaac | 1440 |
| cagatacgct tcaagcaccg cgcgttcctc ccgttcgtgc agcggatcgc caacctgtgc | 1500 |
| atgtccgcca ccctcctata ccatggcaag aagaatggct tcagcaatag cctggagcta | 1560 |
| aaggatatgt tcgagagcca ttttgttaac ccaatcccgc ttaatcatat agattacgat | 1620 |
| taa 1623 | |

Farnesol synthase (AF529266) (Zea mays) (SEQ ID NO: 9)

| | |
|---|---|
| atggccatgc cagtgaagct gactcctgcc tccctctcgc tgaaggcggt ctgctgccgc | 60 |
| ttcagctccg gagggcatgc gctgcgcttc ggctcgtcgc taccgtgctg gaggaggacg | 120 |
| ccgacgcaac ggagcacgtc gtcgtctacg acgcgccctg cggctgaggt tagctctggc | 180 |
| aaaagcaagc agcacgatca agaagcatcg gaggctacga taagacagca gctccagcta | 240 |
| gtcgatgtgc ttgagaacat ggggatttct cggcattttg ctgctgaaat caaatgcatc | 300 |
| cttgacagga catacagaag ttggttacag agacatgagg aaattatgct ggacacaatg | 360 |
| acctgtgcga tggcatttcg tattctaagg ttgaatggat acaatgtctc ttctgatgag | 420 |
| ttgtatcatg ttgttgaagc ttccggactc cataattcac ttggaggata tctcaatgat | 480 |
| acaagaacct tgttagaatt acacaaggcc tcgacagtta gtatctctga agatgagtct | 540 |
| atcctggata gcataggctc aaggtcacgt accttactga gggaacaact agagtctggt | 600 |
| ggtgctctac gaaaaccttc actctttaaa gaggtggaac atgctctgga cggtcccttc | 660 |
| tacaccacat tggaccgtct acaccatagg tggaacatcg aaaatttcaa tattatagag | 720 |
| cagcacatgc tagagacacc atacttgtca aatcaacata ccagtagaga tattctagcg | 780 |
| ttgagtatta gagacttcag ttcctctcag tttacttacc agcaagaact tcaacatctt | 840 |
| gaaagctggg tgaaagagtg caggttagac cagctacaat ttgcgcgaca gaagttggca | 900 |
| tacttctact tgtctgctgc tggcaccatg ttctctcctg agctgtctga tgctcgaact | 960 |
| ttgtgggcca aaaatggtgt gctcacaact attgttgacg acttctttga tgttgcggga | 1020 |
| tcaaaagaag aacttgaaaa ccttgtcatg ttggttgaga tgtgggacga gcatcacaaa | 1080 |
| gttgagttct actcagaaca agtagagatt atattttctt caatttatga ctcagttaac | 1140 |
| caacttggtg aaaaggcttc tttggtacaa gaccgcagta ttaccaaaca cctagtagaa | 1200 |
| atatggttgg atttgctaaa gtctatgatg acagaggtag agtggcgttt gagcaaatat | 1260 |
| gtgccaacag agaaggaata catgataaat gcatctttaa tatttggact aggccccatt | 1320 |
| gtattgccag cattatattt tgttgggcca aagatctcag agtctattgt taaagatcca | 1380 |
| gaatatgatg aattgttcaa actgatgagc acatgtggtc gcctcttgaa tgatgttcag | 1440 |
| acttttgaga gggagtacaa cgagggcaag ttgaatagtg tttctctcct cgttcttcat | 1500 |
| ggtggcccca tgtccatatc agacgccaaa aggaaattac agaagcccat agacacatgc | 1560 |
| agaagagacc tcctaagttt agttcttcgt gaagaaagtg ttgttcctag gccctgcaag | 1620 |
| gaattatttt ggaaaatgtg caaggtgtgc tacttcttct actcgacgac ggatgggttt | 1680 |
| agctcacaag tggagagggc taaagaagtg gatgcggtga tcaatgagcc actaaagcta | 1740 |
| caaggaagtc atacgctggt gtctgatgtg tga | 1773 |

ispA from E. coli (SEQ ID NO: 3)

| | |
|---|---|
| atggactttc cgcagcaact cgaagcctgc gttaagcagg ccaaccaggc gctgagccgt | 60 |
| tttatcgccc cactgccctt tcagaacact cccgtggtcg aaaccatgca gtatggcgca | 120 |
| ttattaggtg gtaagcgcct gcgacctttc ctggtttatg ccactggtca tatgttcggc | 180 |
| gttagcacaa acacactgga cgcacccgct gccgccgtag agtgtatcca cgcttactca | 240 |
| ttaattcatg atgatttacc ggcgatggat gatgacgatc tgcgtcgcgg tttgccgacc | 300 |
| tgccatgtga agtttggcga agcaaacgcg attctcgctg gtgacgcttt acaaacgctg | 360 |
| gcgttctcga ttctaagcga tgccgatatg ccggaagtgt cagatcgcga cagaatttcg | 420 |
| atgatttctg agctggcgag cgccagcggt attgccggaa tgtgcggtgg tcaggcatta | 480 |
| gatttagacg ctgaaggcaa acacgtacct ctggacgcgc ttgagcgtat tcatcgtcat | 540 |
| aaaaccggcg cattgattcg cgccgccgtt cgccttggtg cattaagcgc cggagataaa | 600 |
| ggacgtcgtg ctctgccagt actcgacaag tacgcagaga gcatcggcct tgccttccag | 660 |
| gttcaggatg acatcctgga tgtggtggga gatactgcaa cgttgggaaa acgccagggt | 720 |
| gccgaccagc aacttggtaa aagtacctac cctgcacttc tgggtcttga gcaagcccgg | 780 |
| aagaaagccc gggatctgat cgacgatgcc cgtcagtcgc tgaaacaact ggctgaacag | 840 |
| tcactcgata cctcggcact ggaagcgcta gcggactaca tcatccagcg taataaataa | 900 |

UGT72E1 (Arabidopsis thaliana) (SEQ ID NO: 5)

| | |
|---|---|
| aaaccgacac aataacaaca acaacaacaa caaaaaaaaa aaaaaaaaag gccaacgttc | 60 |
| agtgatctag gctatgaaga ttacaaaacc acatgtggcc atgttcgcta gccccggaat | 120 |
| gggccacatc atcccggtga tcgagctcgg aaaacgctta gctggttccc acggcttcga | 180 |
| tgtcaccatt ttcgtccttg aaaccgacgc agcctcagct caatctcaat tccttaactc | 240 |
| accaggctgc gacgcggccc ttgttgatat cgttggcctc ccaacgcccg atatctccgg | 300 |
| tttagtcgac ccatcagcct tttttgggat caagctcttg gtcatgatgc gtgagaccat | 360 |
| tcctaccatc cggtcaaaga tagaggagat gcaacacaaa ccaacggctc tgatcgtaga | 420 |
| cttgtttggt ttggacgcga taccgctcgg tggtgagttc aacatgttga cttatatctt | 480 |
| catcgcttca aacgcacgtt ttctcgcggt ggctttgttt ttcccaacgt tggacaaaga | 540 |
| catggaagaa gagcacataa tcaagaagca acctatggtt atgcctggat gtgaaccggt | 600 |
| tcggtttgaa gatacacttg aaacattcct tgacccaaac agccaactct accgggaatt | 660 |
| tgttcctttc ggttcggttt tcccaacgtg tgatggtatt attgtgaata catgggatga | 720 |
| tatggagccc aaaactttga aatctcttca agacccaaag ctcttgggtc gaattgctgg | 780 |
| tgtaccggtt tatccaattg gtcctttgtc tagaccggtt gatccatcta aaactaatca | 840 |
| tccggttttg gattggttaa acaaacagcc ggacgagtcg gtactttaca tttcatttgg | 900 |
| aagcggtggc tctctctcgg ctaaacaact aaccgaattg gcttggggac ttgagatgag | 960 |
| tcagcaacgg ttcgtttggg tggttcgacc cccggtggac ggttcagctt gcagtgcata | 1020 |
| tttatccgct aacagtggta aaatacgaga cggtacacct gattatctcc cggaaggttt | 1080 |
| tgttagccgg actcatgaga gaggctttat ggtctcttct tgggctcccc aagcggagat | 1140 |
| cttggcccac caagccgtag gtgggtttct aactcactgc ggttggaatt cgattctcga | 1200 |
| gagcgtcgtt ggtggcgttc cgatgatcgc gtggccactt tttgcggagc agatgatgaa | 1260 |
| cgcgacactc ctcaacgaag agcttggcgt tgccgtccgc tctaagaaac taccgtcgga | 1320 |
| gggagtgatt acgagggcgg agatcgaggc gttggtgaga aagatcatgg tggaggagga | 1380 |
| aggtgctgag atgagaaaga agataaagaa gctgaaagag accgctgccg aatcgctgag | 1440 |
| ttgcgacggt ggagtggcgc atgaatcgtt gtcaagaatc gccgacgaga gcgagcatct | 1500 |
| tttggagcgt gtcaggtgca tggcacgtgg tgcctaggaa cgcttaccgt ttatagtatg | 1560 |
| ttacttataa actgcgtgtc gttttaccct tttgggtatc ttgtattata tagtttccga | 1620 |
| gtcttggttg ccacatggta tcacgtggtt ttgttttggt caatgctagt ccctatcagt | 1680 |
| aatgtatttt acaatgtaaa ataaataatt agtgtttaat ttgt | 1724 |

UGT88A1 (At3g16520/AY037255) (Arabidopsis thaliana) (SEQ ID NO: 11)

| | |
|---|---|
| aaccttagga agagccatgg gtgaagaagc tatagttctg tatcctgcac caccaatagg | 60 |
| tcacttagtg tccatggttg agttaggtaa aaccatcctc tccaaaaacc catctctctc | 120 |
| catccacatt atcttagttc caccgcctta tcagccggaa tcaaccgcca cttacatctc | 180 |
| ctccgtctcc tcctccttcc cttcaataac cttccaccat cttcccgccg tcacaccgta | 240 |
| ctcctcctcc tccacctctc gccaccacca cgaatctctc ctcctagaga tcctctgttt | 300 |
| tagcaaccca agtgtccacc gaactctttt ctcactctct cggaatttca atgtccgagc | 360 |
| aatgatcatc gatttcttct gcaccgccgt tttagacatc accgctgact tcacgttccc | 420 |
| ggtttacttc ttctacacct ctggagccgc atgtctcgcc ttttccttct atctcccgac | 480 |
| catcgacgaa acaacccccg gaaaaaacct caaagacatt cctacagttc atatccccgg | 540 |
| cgttcctccg atgaagggct ccgatatgcc taaggcggtg ctcgaacgag acgatgaggt | 600 |
| ctacgatgtt tttataatgt tcggtaaaca gctctcgaag tcgtcaggga ttattatcaa | 660 |
| tacgtttgat gctttagaaa acagagccat caaggccata acagaggagc tctgttttcg | 720 |
| caatatttat ccaattggac cgctcattgt aaacggaaga atcgaagata gaaacgacaa | 780 |
| caaggcagtt tcttgtctca attggctgga ttcgcagccg gaaaagagtg ttgtgtttct | 840 |
| ctgttttgga agcttaggtt tgttctcaaa agaacaggtg atagagattg ctgttggttt | 900 |
| agagaaaagt gggcagagat tcttgtgggt ggtccgtaat ccacccgagt tagaaaagac | 960 |
| agaactggat ttgaaatcac tcttaccaga aggattctta agccgaaccg aagacaaagg | 1020 |
| gatggtcgtg aaatcatggg ctccgcaagt tccggttctg aatcataaag cagtcggggg | 1080 |
| attcgtcact cattgcggtt ggaattcaat tcttgaagct gtttgtgctg gtgtgccgat | 1140 |
| ggtggcttgg ccgttgtacg ctgagcagag gtttaataga gtgatgattg tggatgagat | 1200 |
| caagattgcg atttcgatga atgaatcaga gacgggtttc gtgagctcta cagaggtgga | 1260 |
| gaaacgagtc caagagataa ttggggagtg tccggttagg gagcgaacca tggctatgaa | 1320 |
| gaacgcagcc gaattagcct tgacagaaac tggttcgtct cataccgcat taactacttt | 1380 |
| actccagtcg tggagcccaa agtgatcttt gaaattaagt cttgcatcaa ttaataactt | 1440 |
| tgtagtctct cagaatcatg tctttggggg ttattcgttt tactagttca aagttatgtc | 1500 |
| tctgagaccg cagatggaaa gaacaattta tacc | 1534 |

UTG85A4 (At1g78270) (Arabidopsis thaliana) (SEQ ID NO: 13)

| | |
|---|---|
| atggaacaac atggcggttc tagctcacag aaacctcacg caatgtgcat accttatcca | 60 |
| gcacaaggcc acatcaaccc aatgctgaaa ctagccaagc tcctccacgc tagaggcttc | 120 |
| cacgtcactt tcgtcaacac cgactacaac caccgccgta tcctccaatc acgtggccct | 180 |
| cacgctctca acggtctccc ctcgtttcgc ttcgagacta tccccgacgg tcttccttgg | 240 |
| acagacgtcg acgctaagca agacatgctc aagcttattg actccacaat aaacaactgt | 300 |
| ttagctccat tcaaagacct catcctccgg ttaaactccg gttctgatat accaccggtt | 360 |
| agctgtatca tctccgacgc ttcaatgagc ttcacaattg acgcagcgga ggagcttaaa | 420 |
| attccggtag ttctcctctg gaccaacagt gctactgctt taatcttgta tctccattac | 480 |
| caaaaactca tcgagaaaga gataattccc ctcaaagatt cgagtgactt gaagaagcat | 540 |
| ttagagacgg agattgattg gataccgtcg atgaagaaga ttaagcttaa ggattttcca | 600 |
| gatttcgtca ccacgacgaa tcctcaagat ccgatgatta gtttcatcct tcatgtaacc | 660 |
| ggaagaatca aaagagcttc tgcgatcttc atcaacactt tcgaaaaact cgagcataac | 720 |
| gttctcttat ctctgcgatc tcttctccct cagatctact ccgttggacc gttccagatt | 780 |
| ctggagaatc gcgaaatcga taagaacagc gaaatcagaa agctaggatt gaatctctgg | 840 |
| gaagaagaga cggagtcttt ggattggcta gatactaaag ctgagaaagc tgtgatttac | 900 |
| gtcaacttcg ggagtctaac ggttttgact agtgagcaga tcttagagtt cgcttggggt | 960 |
| ttagcgagga gcgggaaaga gtttctctgg gtggtgagat ctggtatggt cgacggagat | 1020 |
| gattcgattc ttccggcgga gtttttatcg gagacgaaga atcgaggaat gttaattaaa | 1080 |
| ggatggtgtt ctcaggagaa ggtactttcg catccggcga ttggaggatt tttgactcac | 1140 |
| tgtggatgga attcgacgtt ggagagtttg tacgccggtg ttccgatgat ctgttggcca | 1200 |
| ttttttgctg atcagttgac gaatcgaaag ttctgttgcg aggattgggg gattgggatg | 1260 |
| gagatcggcg aggaggtgaa gagggagaga gtggagacgg tggttaaaga gctcatggac | 1320 |
| ggagagaagg gaaagaggtt aagagagaag gtggtggagt ggcggcgctt ggcggaagaa | 1380 |
| gcttcggcgc caccgttggg atcatcgtac gtgaattttg aaacggtggt taataaagtc | 1440 |
| cttacatgtc acacgattag atcgacctaa ttgggctttc cgtatccaaa taggcccatt | 1500 |
| agaaaaggcc tgtttgtttc cactatcatt tggg | 1534 |

UTG85A2 (At1g22360/AF332418) (Arabidopsis thaliana) (SEQ ID NO: 15)

| | |
|---|---|
| atgggatctc atgtcgcaca aaaacaacac gtagtttgcg ttccttatcc ggctcaaggc | 60 |
| cacatcaacc caatgatgaa agtggctaaa ctcctttacg ccaaaggctt ccatattacc | 120 |
| ttcgtcaaca ccgtctacaa ccacaaccgt ctcctccggt cccgtgggcc taacgccgtt | 180 |
| gacgggcttc cttctttccg gtttgagtcc atccctgacg gtctacccga gactgacgta | 240 |
| gacgtcactc aggacatccc tactctttgc gagtccacaa tgaagcactg tctcgctcca | 300 |
| ttcaaggagc ttctccggca gatcaacgca agggatgatg ttcctcctgt gagctgtatc | 360 |
| gtatccgacg gttgtatgag cttcacactt gatgctgcgg aggagctcgg tgtcccggag | 420 |
| gttctttttt ggacaactag tgcttgtggc ttcttggctt acctttacta ctatcgcttc | 480 |
| atcgagaagg gattatcacc aataaaagat gagagttact taaccaagga acacttggac | 540 |
| acaaaaatag actggatacc atcgatgaag gacctaagac taaaagacat ccctagcttc | 600 |
| atccgaacga ctaatcctga cgacatcatg ctcaacttta tcatccgtga ggctaaccga | 660 |
| gccaaacgcg cttcagctat cattctcaac acgtttgatg atctcgaaca cgacgttatc | 720 |
| caatctatga aatccattgt acctccggtt tattctattg gaccgttaca tttactagag | 780 |
| aaacaagaga gcggcgagta tagtgaaatc ggacggacag gatcgaatct ttggagagag | 840 |
| gagactgagt gtctggactg gctaaacacg aaagctagaa acagtgttgt gtacgttaac | 900 |
| ttcgggagta taactgtttt gagcgcaaaa cagcttgtgg agtttgcatg gggtttggct | 960 |
| gcaacgggga aagagttttt gtgggtgatc cggccggatt tagtagccgg ggatgaggca | 1020 |
| atggttccac cggagttttt aacggctacg gcggaccgga ggatgttggc aagttggtgt | 1080 |
| cctcaagaga aagtcctttc tcatccggcc attggagggt tcttgacgca ttgcgggtgg | 1140 |
| aactcgacgt tggaaagtct atgcggtgga gttccaatgg tgtgttggcc gttttttgca | 1200 |
| gagcaacaaa ctaattgtaa gttttctcgt gacgaatggg aggttgggat tgagattggt | 1260 |
| ggagatgtga agagagaaga ggttgaggcg gtggttaggg agttgatgga tgaagagaag | 1320 |
| ggaaagaata tgagagagaa ggcggaagag tggcggcgct tggcgaatga agcgacggag | 1380 |
| cataagcatg gttcttctaa attgaacttt gagatgctcg ttaataaggt tcttttaggg | 1440 |
| gagtagagac tagtaaatat ggatttcaag aagaaagg | 1478 |

UTG85A1 (At1g22400) (Arabidopsis thaliana) (SEQ ID NO: 17)

| | |
|---|---|
| aaaactcaca tttgttcatt acaaaaagag gaaactgaca actgatttcg cgcaagagaa | 60 |
| agaaaaatgg gatctcagat cattcataac tcacaaaaac cacatgtagt ttgtgttcca | 120 |
| tatccggctc aaggccacat caaccctatg atgagagtgg ctaaactcct ccacgccaga | 180 |
| ggcttctacg tcaccttcgt caacaccgtc tacaaccaca atcgtttcct tcgttctcgt | 240 |
| gggtccaatg ccctagatgg acttccttcg ttccgatttg agtccattgc tgacggtcta | 300 |
| ccagagacag acatggatgc cacgcaggac atcacagctc tttgcgagtc caccatgaag | 360 |
| aactgtctcg ctccgttcag agagcttctc cagcggatca acgctggaga taatgttcct | 420 |
| ccggtaagct gtattgtatc tgacggttgt atgagcttta ctcttgatgt tgcggaggag | 480 |
| cttggagtcc cggaggttct tttttggaca accagtggct gtgcgttcct ggcttatcta | 540 |
| cacttttatc tcttcatcga gaagggctta tgtccgctaa aagatgagag ttacttgacg | 600 |
| aaggagtact tagaagacac ggttatagat tttataccaa ccatgaagaa tgtgaaacta | 660 |
| aaggatattc ctagcttcat acgtaccact aatcctgatg atgttatgat tagtttcgcc | 720 |
| ctccgcgaga ccgagcgagc caaacgtgct tctgctatca ttctaaacac atttgatgac | 780 |
| cttgagcatg atgttgttca tgctatgcaa tctatcttac ctccggttta ttcagttgga | 840 |
| ccgcttcatc tcttagcaaa ccgggagatt gaagaaggta gtgagattgg aatgatgagt | 900 |
| tcgaatttat ggaaagagga gatggagtgt ttggattggc ttgatactaa gactcaaaat | 960 |
| agtgtcattt atatcaactt tgggagcata acggttttga gtgtgaagca gcttgtggag | 1020 |
| tttgcttggg gtttggcggg aagtgggaaa gagtttttat gggtgatccg gccagattta | 1080 |
| gtagcgggag aggaggctat ggttccgccg gactttttaa tggagactaa agaccgcagt | 1140 |
| atgctagcga gttggtgtcc tcaagagaaa gtactttctc atcctgctat tggagggttt | 1200 |
| ttgacgcatt gcgggtggaa ctcgatattg gaaagtcttt cgtgtggagt tccgatggtg | 1260 |
| tgttggccat tttttgctga ccagcaaatg aattgtaagt tttgttgtga cgagtgggat | 1320 |
| gttgggattg agataggtgg agatgtgaag agagaggaag ttgaggcggt ggttagagag | 1380 |
| ctcatggatg gagagaaggg aaagaaaatg agagaaaagg cggtagagtg gcagcgctta | 1440 |
| gccgagaaag cgacggaaca taaacttggt tcttccgtta tgaattttga gacggttgtt | 1500 |
| agcaagtttc ttttgggaca aaaatcacag gattaactaa aatataagat gatcatcgaa | 1560 |
| atgtattatt ttttctataa tgattactaa gtaagtgttt atttt | 1605 |

UTG85A7 (At1g22340) (Arabidopsis thaliana) (SEQ ID NO: 19)

| | |
|---|---|
| atggaatctc atgttgttca taacgcacaa aagccacacg tagtttgcgt gccttacccg | 60 |
| gctcaaggcc acatcaatcc gatgctgaaa gtggctaaac tcctctacgc taaaggcttt | 120 |
| cacgtcacct tcgttaacac tctctacaac cacaaccgtc tcctccggtc ccgtggtccc | 180 |
| aacgcgctcg acgggtttcc ttcattccgg ttcgagtcca tccctgacgg tctaccggag | 240 |
| actgatggcg ataggacgca gcatactcct accgtttgca tgtccattga gaaaaactgt | 300 |
| ctcgctccat tcaaagagat tctgcgccgg atcaacgata aagatgatgt tcctccagtg | 360 |
| agttgtattg tatcggacgg tgtgatgagt tttactcttg acgcagccga ggaactaggt | 420 |
| gtcccagagg ttattttttg gaccaatagt gcttgtggtt tcatgactat tctacacttt | 480 |
| tatcttttca tcgagaaggg tctatctcct tttaaagacg aaagttacat gtcaaaggag | 540 |
| catctagaca cagttataga ttggatacca tcaatgaaga atcttaggtt aaaggacatc | 600 |
| cctagctata tacgtaccac aaatcctgac aacataatgc ttaatttcct cattcgagaa | 660 |
| gttgagcgat ctaaacgcgc tagtgctatc attctcaaca cgtttgatga actcgagcat | 720 |
| gatgttatcc agtctatgca atctatttta cctccggttt attctattgg gccactccat | 780 |
| ctccttgtga aggaagaaat aaacgaggct agtgaaatag gacagatggg attaaatttg | 840 |
| tggagagagg agatggaatg tttggattgg ctcgatacaa aaactccaaa cagtgttctt | 900 |
| tttgttaact ttggatgcat aacggtgatg agtgcaaaac agcttgaaga atttgcttgg | 960 |
| ggtttggcgg caagtaggaa agagttttta tgggtgatcc gtcctaattt agtggtggga | 1020 |
| gaggcgatgg tggttcttcc acaagagttt ttagcggaga cgatagaccg gagaatgtta | 1080 |
| gctagttggt gtcctcagga gaaagttctt tctcatcccg cgataggagg gttcttgacg | 1140 |
| cattgcgggt ggaactcaac attggagagt ctcgctggtg gtgttccgat gatatgttgg | 1200 |
| ccatgttttt cggagcaacc gacgaattgt aagttttgtt gtgacgagtg gggagtgggt | 1260 |
| atagagattg gtaaagatgt gaagagagag gaggtcgaga cggtggttag agaacttatg | 1320 |
| gatggagaaa aggggaaaaa gctgagagaa aaggcggaag agtggcggcg gttggccgag | 1380 |
| gaagcgacga ggtataaaca tggttcgtcg gtcatgaatc ttgagacgct tatacataaa | 1440 |
| gttttcttag aaaatcttag atga | 1464 |

UTG73C6 (At2g36790) (Arabidopsis thaliana) (SEQ ID NO: 21)

| | |
|---|---|
| gaaacaaaac ttgagaggtt cttactaaag ttgcatcgtc atggctttcg aaaaaaacaa | 60 |
| cgaacctttt cctcttcact ttgttctctt ccctttcatg gctcaaggcc acatgattcc | 120 |
| catggttgat attgcaaggc tcttggctca gcgaggtgtg cttataacaa ttgtcacgac | 180 |
| gcctcacaat gcagcaaggt tcaagaatgt cctaaaccgt accattgagt ctggtttgcc | 240 |
| catcaaccta gtgcaagtca agtttccata tcaagaagct ggtctgcaag aaggacaaga | 300 |
| aaatatggat ttgcttacca cgatggagca gataacatct ttctttaaag cggttaactt | 360 |
| actcaaagaa ccagtccaga accttattga agagatgagc ccgcgaccaa gctgtctaat | 420 |
| ctctgatatg tgtttgtcgt atacaagcga aatcgccaag aagttcaaaa taccaaagat | 480 |
| cctcttccat ggcatgggtt gcttttgtct tctgtgtgtt aacgttctgc gcaagaaccg | 540 |
| tgagatcttg gacaatttaa agtctgataa ggagtacttc attgttcctt attttcctga | 600 |
| tagagttgaa ttcacaagac ctcaagttcc ggtggaaaca tatgttcctg caggctggaa | 660 |
| agagatcttg gaggatatgg tagaagcgga taagacatct tatggtgtta tagtcaactc | 720 |
| atttcaagag ctcgaacctg cgtatgccaa agacttcaag gaggcaaggt ctggtaaagc | 780 |
| atggaccatt ggacctgttt ccttgtgcaa caaggtagga gtagacaaag cagagagggg | 840 |
| aaacaaatca gatattgatc aagatgagtg ccttgaatgg ctcgattcta aggaaccggg | 900 |
| atctgtgctc tacgtttgcc ttggaagtat ttgtaatctt cctctgtctc agctccttga | 960 |
| gctgggacta ggcctagagg aatcccaaag acctttcatc tgggtcataa gaggttggga | 1020 |
| gaaatacaaa gagttagttg agtggttctc ggaaagcggc tttgaagata gaatccaaga | 1080 |
| tagaggactt ctcatcaaag gatggtcccc tcaaatgctt atcctttcac atccttctgt | 1140 |
| tggagggttc ttaacgcact gcggatggaa ctcgactctt gaggggataa ctgctggtct | 1200 |
| accaatgctt acatggccac tatttgcaga ccaattctgc aacgagaaac tggtcgtaca | 1260 |
| aatactaaaa gtcggtgtaa gtgccgaggt taaagaggtc atgaaatggg gagaagaaga | 1320 |
| gaagatagga gtgttggtgg ataaagaagg agtgaagaag gcagtggaag aactaatggg | 1380 |
| tgagagtgat gatgcaaaag agagaagaag aagagccaaa gagcttggag aatcagctca | 1440 |
| caaggctgtg gaagaaggag gctcctctca ttctaatatc actttcttgc tacaagacat | 1500 |
| aatgcaacta gcacagtcca ataattgagt atatgtcata ttttcaaagg aatttaaaca | 1560 |
| ttctatagtt tttgttttct gtatttgtga aatttaaaac agagtcttag tt | 1612 |

UTG73C5 (At2g36800) (Arabidopsis thaliana) (SEQ ID NO: 23)

| | |
|---|---|
| atggtttccg aaacaaccaa atcttctcca cttcactttg ttctcttccc tttcatggct | 60 |
| caaggccaca tgattcccat ggttgatatt gcaaggctct tggctcagcg tggtgtgatc | 120 |
| ataacaattg tcacgacgcc tcacaatgca gcgaggttca agaatgtcct aaaccgtgcc | 180 |
| attgagtctg gcttgcccat caacttagtg caagtcaagt ttccatatct agaagctggt | 240 |
| ttgcaagaag gacaagagaa tatcgattct cttgacacaa tggagcggat gatacctttc | 300 |
| tttaaagcgg ttaactttct cgaagaacca gtccagaagc tcattgaaga gatgaaccct | 360 |
| cgaccaagct gtctaatttc tgatttttgt ttgccttata caagcaaaat cgccaagaag | 420 |
| ttcaatatcc caaagatcct cttccatggc atgggttgct tttgtcttct gtgtatgcat | 480 |
| gttttacgca agaaccgtga gatcttggac aatttaaagt cagataagga gcttttcact | 540 |
| gttcctgatt ttcctgatag agttgaattc acaagaacgc aagttccggt agaaacatat | 600 |
| gttccagctg gagactggaa agatatcttt gatggtatgg tagaagcgaa tgagacatct | 660 |
| tatggtgtga tcgtcaactc atttcaagag ctcgagcctg cttatgccaa agactacaag | 720 |
| gaggtaaggt ccggtaaagc atggaccatt ggacccgttt ccttgtgcaa caaggtagga | 780 |
| gccgacaaag cagagagggg aaacaaatca gacattgatc aagatgagtg ccttaaatgg | 840 |
| ctcgattcta agaaacatgg ctcggtgctt tacgtttgtc ttggaagtat ctgtaatctt | 900 |
| cctttgtctc aactcaagga gctgggacta ggcctagagg aatcccaaag acctttcatt | 960 |
| tgggtcataa gaggttggga gaagtacaaa gagttagttg agtggttctc ggaaagcggc | 1020 |
| tttgaagata gaatccaaga tagaggactt ctcatcaaag gatggtcccc tcaaatgctt | 1080 |
| atcctttcac atccatcagt tggagggttc ctaacacact gtggttggaa ctcgactctt | 1140 |
| gaggggataa ctgctggtct accgctactt acatggccgc tattcgcaga ccaattctgc | 1200 |
| aatgagaaat tggtcgttga ggtactaaaa gccggtgtaa gatccggggt tgaacagcct | 1260 |
| atgaaatggg gagaagagga gaaaatagga gtgttggtgg ataaagaagg agtgaagaag | 1320 |
| gcagtggaag aattaatggg tgagagtgat gatgcaaaag agagaagaag aagagccaaa | 1380 |
| gagcttggag attcagctca caaggctgtg gaagaaggag gctcttctca ttctaacatc | 1440 |
| tctttcttgc tacaagacat aatggaactg gcagaaccca ataattgagt atacgtcatc | 1500 |
| tttttaaagg aatttaaaaa ttaaatagtt ttgttttctg tatttgtgaa | 1550 |

Linalool synthase (Arabidopsis thaliana) (SEQ ID NO: 7)

| | |
|---|---|
| atggccttaa tagctaccaa aataagttca cgttcttgtt ttgtttctgc ttatccaaac | 60 |
| aattctccaa cgtttctgat atccaaattt cccaatacgg tcgattcttt gtcgcctgca | 120 |
| aatactgcta aaagatcgat cttgagaaac gtccatgctt cagtttctaa cccttcgaag | 180 |
| cagtttcata ataagacatc cttagaatat tcacacgagt tgaatatcaa gaagatcaag | 240 |
| aatatactaa gtgcaaacgt agatgttcca tccgagaacc tggagatgat cgatgtcatt | 300 |
| caaagtttag gcactgatct ccattttcga caagggatcg agcaaaccct tcacatgatt | 360 |
| tacaaagaag gcctccaatt caacggtgat ctccatgaga tagcgcttcg ctttcgattg | 420 |
| ctgagacaag agggtcacta tgttcaagaa agtattttca aaaacatcct agacaagaag | 480 |
| ggtggattta aagacgtagt aaaaaatgac gtcaagggtc taacagaatt gtttgaagct | 540 |
| tctgagctcc gtgtagaagg tgaagaaaca ctcgacggtg ccagagaatt cacatatagc | 600 |
| cgccttaatg agctttgctc aggtagagaa agtcatcaaa agcaagagat aatgaagtct | 660 |
| ttggcgcaac ctcgccacaa aaccgtaaga ggattaacgt ccaagaggtt cacaagcatg | 720 |
| atcaaaatcg cgggtcaaga agatccagaa tggttacagt ctctattacg agtggcggag | 780 |
| atcgattcca ttaggctaaa gtcattgact caaggagaaa tgtctcaaac atttaaatgg | 840 |
| tggacagaac ttggtttaga aaaagatgtg gagaaggcaa gaagccagcc gttaaaatgg | 900 |
| catacgtggt ccatgaaaat tcttcaagat ccgaccttaa ccgaacaaag gcttgatctt | 960 |
| accaaaccaa tatcgcttgt ttatgttata gatgacattt tcgatgtcta tggggagcta | 1020 |
| gaagaactaa ccatcttcac acgagttgtt gagagatggg atcataaggg gcttaagacg | 1080 |
| ctacccaaat acatgagggt ttgttttgaa gctctagata tgatcacaac ggagattagc | 1140 |
| atgaagatct acaaatcaca tggttggaac ccgacatacg ctcttcgaca atcgtgggca | 1200 |
| agtttgtgta aagcattctt ggtagaagca aagtggttta attcgggtta cttacccacc | 1260 |
| actgaagagt atatgaagaa tggggttgtg agttcaggtg ttcatttagt gatgcttcat | 1320 |
| gcctatatct tgttaggcga agaactaaca aaagagaaag tcgaactaat agagagtaac | 1380 |
| ccggggattg tatcatctgc agctacaatt ctcaggctct gggatgatct cggaagtgcc | 1440 |
| aaggatgaga accaagatgg aactgatgga tcatatgtag agtgttacct gaacgagtac | 1500 |
| aagggatcaa ctgttgatga agcaagaaca catgttgccc agaagatatc tagagcatgg | 1560 |
| aaacgcttga acagggagtg tctgaatcca tgtccattct caagatcatt ctcaaaagct | 1620 |
| tgtctcaaca ttgcaagaac agttccttta atgtacagct atgatgatga tcaacgactt | 1680 |
| cccgacgaat atctcaagtc tctaatgtaa | 1710 |

TABLE 2
Exemplary polypeptides

Farnesol synthase (OsTPS13) (Oryza sativa) (SEQ ID NO: 2)

| | |
|---|---|
| MAPAFHPAIF GDFFINNVQP SPKESDEWME ERVDQLVEEV GRMLEVCKDD VVKQMNLVDV | 60 |
| LQRLGIDHHF EEQIDTILKN IHRAEFNSSD LYEVALRFRL LRKQGYWVSP DEFNKFKAED | 120 |
| GSFSSDDITN DPKGLLSLYN AAHLLTHNEK ALEEAILFAR HHLQLLRGNL AYPLDEQVTR | 180 |
| ALEIPLPRTM KRVEVLNYIF EYSAEEKMFN PSILELAVLD FNILQKVHQN ELKEICQWWE | 240 |
| NLSSDIRLDY VRERVVECYF CAYAAYYEKE HARARMIFAK RCMLFSLLDD TYDVRATLEE | 300 |
| ARKFNDALQR WDKSDVSLLP EDLKRFFLSI ISNFREFEDE LEPHEKYRNS YNIKAFQILS | 360 |
| SNFLQEAEWF HQNYIPCFTD HVTVSLQTGG AIELPVSLIV GMGDIATKEV LDWALANPDA | 420 |
| GRAFAEVARF MDDLAASHSG RDKMDVASTV ECYMNEHGVT REVAEAKIAG MAEDGWKSMN | 480 |
| QIRFKHRAFL PFVQRIANLC MSATLLYHGK KNGFSNSLEL KDMFESHFVN PIPLNHIDYD | 540 |

Farnesol synthase (AF529266) (Zea mays) (SEQ ID NO: 10)

| | |
|---|---|
| MAMPVKLTPA SLSLKAVCCR FSSGGHALRF GSSLPCWRRT PTQRSTSSST TRPAAEVSSG | 60 |
| KSKQHDQEAS EATIRQQLQL VDVLENMGIS RHFAAEIKCI LDRTYRSWLQ RHEEIMLDTM | 120 |
| TCAMAFRILR LNGYNVSSDE LYHVVEASGL HNSLGGYLND TRTLLELHKA STVSISEDES | 180 |
| ILDSIGSRSR TLLREQLESG GALRKPSLFK EVEHALDGPF YTTLDRLHHR WNIENFNIIE | 240 |
| QHMLETPYLS NQHTSRDILA LSIRDFSSSQ FTYQQELQHL ESWVKECRLD QLQFARQKLA | 300 |
| YFYLSAAGTM FSPELSDART LWAKNGVLTT IVDDFFDVAG SKEELENLVM LVEMWDEHHK | 360 |
| VEFYSEQVEI IFSSIYDSVN QLGEKASLVQ DRSITKHLVE IWLDLLKSMM TEVEWRLSKY | 420 |
| VPTEKEYMIN ASLIFGLGPI VLPALYFVGP KISESIVKDP EYDELFKLMS TCGRLLNDVQ | 480 |
| TFEREYNEGK LNSVSLLVLH GGPMSISDAK RKLQKPIDTC RRDLLSLVLR EESVVPRPCK | 540 |
| ELFWKMCKVC YFFYSTTDGF SSQVERAKEV DAVINEPLKL QGSHTLVSDV | 590 |

ispA (E. coli) (SEQ ID NO: 4)

| | |
|---|---|
| MDFPQQLEAC VKQANQALSR FIAPLPFQNT PVVETMQYGA LLGGKRLRPF LVYATGHMFG | 60 |
| VSTNTLDAPA AAVECIHAYS LIHDDLPAMD DDDLRRGLPT CHVKFGEANA ILAGDALQTL | 120 |
| AFSILSDADM PEVSDRDRIS MISELASASG IAGMCGGQAL DLDAEGKHVP LDALERIHRH | 180 |
| KTGALIRAAV RLGALSAGDK GRRALPVLDK YAESIGLAFQ VQDDILDVVG DTATLGKRQG | 240 |
| ADQQLGKSTY PALLGLEQAR KKARDLIDDA RQSLKQLAEQ SLDTSALEAL ADYIIQRNK | 299 |

UGT72E1 (Arabidopsis thaliana) (SEQ ID NO: 6)

| | |
|---|---|
| MKITKPHVAM FASPGMGHII PVIELGKRLA GSHGFDVTIF VLETDAASAQ SQFLNSPGCD | 60 |
| AALVDIVGLP TPDISGLVDP SAFFGIKLLV MMRETIPTIR SKIEEMQHKP TALIVDLFGL | 120 |
| DAIPLGGEFN MLTYIFIASN ARFLAVALFF PTLDKDMEEE HIIKKQPMVM PGCEPVRFED | 180 |
| TLETFLDPNS QLYREFVPFG SVFPTCDGII VNTWDDMEPK TLKSLQDPKL LGRIAGVPVY | 240 |
| PIGPLSRPVD PSKTNHPVLD WLNKQPDESV LYISFGSGGS LSAKQLTELA WGLEMSQQRF | 300 |
| VWVVRPPVDG SACSAYLSAN SGKIRDGTPD YLPEGFVSRT HERGFMVSSW APQAEILAHQ | 360 |
| AVGGFLTHCG WNSILESVVG GVPMIAWPLF AEQMMNATLL NEELGVAVRS KKLPSEGVIT | 420 |
| RAEIEALVRK IMVEEEGAEM RKKIKKLKET AAESLSCDGG VAHESLSRIA DESEHLLERV | 480 |
| RCMARGA | 487 |

UGT88A1 (At3g16520/AY037255 ) (Arabidopsis thaliana) (SEQ ID NO: 12)

| | |
|---|---|
| MGEEAIVLYP APPIGHLVSM VELGKTILSK NPSLSIHIIL VPPPYQPEST ATYISSVSSS | 60 |
| FPSITFHHLP AVTPYSSSST SRHHHESLLL EILCFSNPSV HRTLFSLSRN FNVRAMIIDF | 120 |
| FCTAVLDITA DFTFPVYFFY TSGAACLAFS FYLPTIDETT PGKNLKDIPT VHIPGVPPMK | 180 |
| GSDMPKAVLE RDDEVYDVFI MFGKQLSKSS GIIINTFDAL ENRAIKAITE ELCFRNIYPI | 240 |
| GPLIVNGRIE DRNDNKAVSC LNWLDSQPEK SVVFLCFGSL GLFSKEQVIE IAVGLEKSGQ | 300 |
| RFLWVVRNPP ELEKTELDLK SLLPEGFLSR TEDKGMVVKS WAPQVPVLNH KAVGGFVTHC | 360 |
| GWNSILEAVC AGVPMVAWPL YAEQRFNRVM IVDEIKIAIS MNESETGFVS STEVEKRVQE | 420 |
| IIGECPVRER TMAMKNAAEL ALTETGSSHT ALTTLLQSWS PK | 462 |

UTG85A4 (At1g78270) (Arabidopsis thaliana) (SEQ ID NO: 14)

| | |
|---|---|
| MEQHGGSSSQ KPHAMCIPYP AQGHINPMLK LAKLLHARGF HVTFVNTDYN HRRILQSRGP | 60 |
| HALNGLPSFR FETIPDGLPW TDVDAKQDML KLIDSTINNC LAPFKDLILR LNSGSDIPPV | 120 |
| SCIISDASMS FTIDAAEELK IPVVLLWTNS ATALILYLHY QKLIEKEIIP LKDSSDLKKH | 180 |
| LETEIDWIPS MKKIKLKDFP DFVTTTNPQD PMISFILHVT GRIKRASAIF INTFEKLEHN | 240 |
| VLLSLRSLLP QIYSVGPFQI LENREIDKNS EIRKLGLNLW EEETESLDWL DTKAEKAVIY | 300 |
| VNFGSLTVLT SEQILEFAWG LARSGKEFLW VVRSGMVDGD DSILPAEFLS ETKNRGMLIK | 360 |
| GWCSQEKVLS HPAIGGFLTH CGWNSTLESL YAGVPMICWP FFADQLTNRK FCCEDWGIGM | 420 |
| EIGEEVKRER VETVVKELMD GEKGKRLREK VVEWRRLAEE ASAPPLGSSY VNFETVVNKV | 480 |
| LTCHTIRST | 489 |

UTG85A2 (At1g22360/AF332418) (Arabidopsis thaliana) (SEQ ID NO: 16)

| | |
|---|---|
| MGSHVAQKQH VVCVPYPAQG HINPMMKVAK LLYAKGFHIT FVNTVYNHNR LLRSRGPNAV | 60 |
| DGLPSFRFES IPDGLPETDV DVTQDIPTLC ESTMKHCLAP FKELLRQINA RDDVPPVSCI | 120 |
| VSDGCMSFTL DAAEELGVPE VLFWTTSACG FLAYLYYYRF IEKGLSPIKD ESYLTKEHLD | 180 |
| TKIDWIPSMK DLRLKDIPSF IRTTNPDDIM LNFIIREANR AKRASAIILN TFDDLEHDVI | 240 |
| QSMKSIVPPV YSIGPLHLLE KQESGEYSEI GRTGSNLWRE ETECLDWLNT KARNSVVYVN | 300 |
| FGSITVLSAK QLVEFAWGLA ATGKEFLWVI RPDLVAGDEA MVPPEFLTAT ADRRMLASWC | 360 |
| PQEKVLSHPA IGGFLTHCGW NSTLESLCGG VPMVCWPFFA EQQTNCKFSR DEWEVGIEIG | 420 |
| GDVKREEVEA VVRELMDEEK GKNMREKAEE WRRLANEATE HKHGSSKLNF EMLVNKVLLG | 480 |
| E | 481 |

UTG85A1 (At1g22400) (Arabidopsis thaliana) (SEQ ID NO: 18)

| | |
|---|---|
| MGSQIIHNSQ KPHVVCVPYP AQGHINPMMR VAKLLHARGF YVTFVNTVYN HNRFLRSRGS | 60 |
| NALDGLPSFR FESIADGLPE TDMDATQDIT ALCESTMKNC LAPFRELLQR INAGDNVPPV | 120 |
| SCIVSDGCMS FTLDVAEELG VPEVLFWTTS GCAFLAYLHF YLFIEKGLCP LKDESYLTKE | 180 |
| YLEDTVIDFI PTMKNVKLKD IPSFIRTTNP DDVMISFALR ETERAKRASA IILNTFDDLE | 240 |
| HDVVHAMQSI LPPVYSVGPL HLLANREIEE GSEIGMMSSN LWKEEMECLD WLDTKTQNSV | 300 |
| IYINFGSITV LSVKQLVEFA WGLAGSGKEF LWVIRPDLVA GEEAMVPPDF LMETKDRSML | 360 |
| ASWCPQEKVL SHPAIGGFLT HCGWNSILES LSCGVPMVCW PFFADQQMNC KFCCDEWDVG | 420 |
| IEIGGDVKRE EVEAVVRELM DGEKGKKMRE KAVEWQRLAE KATEHKLGSS VMNFETVVSK | 480 |
| FLLGQKSQD | 489 |

UTG85A7 (At1g22340) (Arabidopsis thaliana) (SEQ ID NO: 20)

| | |
|---|---|
| MESHVVHNAQ KPHVVCVPYP AQGHINPMLK VAKLLYAKGF HVTFVNTLYN HNRLLRSRGP | 60 |
| NALDGFPSFR FESIPDGLPE TDGDRTQHTP TVCMSIEKNC LAPFKEILRR INDKDDVPPV | 120 |
| SCIVSDGVMS FTLDAAEELG VPEVIFWTNS ACGFMTILHF YLFIEKGLSP FKDESYMSKE | 180 |
| HLDTVIDWIP SMKNLRLKDI PSYIRTTNPD NIMLNFLIRE VERSKRASAI ILNTFDELEH | 240 |
| DVIQSMQSIL PPVYSIGPLH LLVKEEINEA SEIGQMGLNL WREEMECLDW LDTKTPNSVL | 300 |
| FVNFGCITVM SAKQLEEFAW GLAASRKEFL WVIRPNLVVG EAMVVLPQEF LAETIDRRML | 360 |
| ASWCPQEKVL SHPAIGGFLT HCGWNSTLES LAGGVPMICW PCFSEQPTNC KFCCDEWGVG | 420 |
| IEIGKDVKRE EVETVVRELM DGEKGKKLRE KAEEWRRLAE EATRYKHGSS VMNLETLIHK | 480 |
| VFLENLR | 487 |

UTG73C6 (At2g36790) (Arabidopsis thaliana) (SEQ ID NO: 22)

| | |
|---|---|
| MAFEKNNEPF PLHFVLFPFM AQGHMIPMVD IARLLAQRGV LITIVTTPHN AARFKNVLNR | 60 |
| TIESGLPINL VQVKFPYQEA GLQEGQENMD LLTTMEQITS FFKAVNLLKE PVQNLIEEMS | 120 |
| PRPSCLISDM CLSYTSEIAK KFKIPKILFH GMGCFCLLCV NVLRKNREIL DNLKSDKEYF | 180 |
| IVPYFPDRVE FTRPQVPVET YVPAGWKEIL EDMVEADKTS YGVIVNSFQE LEPAYAKDFK | 240 |
| EARSGKAWTI GPVSLCNKVG VDKAERGNKS DIDQDECLEW LDSKEPGSVL YVCLGSICNL | 300 |
| PLSQLLELGL GLEESQRPFI WVIRGWEKYK ELVEWFSESG FEDRIQDRGL LIKGWSPQML | 360 |
| ILSHPSVGGF LTHCGWNSTL EGITAGLPML TWPLFADQFC NEKLVVQILK VGVSAEVKEV | 420 |
| MKWGEEEKIG VLVDKEGVKK AVEELMGESD DAKERRRRAK ELGESAHKAV EEGGSSHSNI | 480 |
| TFLLQDIMQL AQSNN | 495 |

UTG73C5 (At2g36800) (Arabidopsis thaliana) (SEQ ID NO: 24)

| | |
|---|---|
| MVSETTKSSP LHFVLFPFMA QGHMIPMVDI ARLLAQRGVI ITIVTTPHNA ARFKNVLNRA | 60 |
| IESGLPINLV QVKFPYLEAG LQEGQENIDS LDTMERMIPF FKAVNFLEEP VQKLIEEMNP | 120 |
| RPSCLISDFC LPYTSKIAKK FNIPKILFHG MGCFCLLCMH VLRKNREILD NLKSDKELFT | 180 |
| VPDFPDRVEF TRTQVPVETY VPAGDWKDIF DGMVEANETS YGVIVNSFQE LEPAYAKDYK | 240 |
| EVRSGKAWTI GPVSLCNKVG ADKAERGNKS DIDQDECLKW LDSKKHGSVL YVCLGSICNL | 300 |
| PLSQLKELGL GLEESQRPFI WVIRGWEKYK ELVEWFSESG FEDRIQDRGL LIKGWSPQML | 360 |
| ILSHPSVGGF LTHCGWNSTL EGITAGLPLL TWPLFADQFC NEKLVVEVLK AGVRSGVEQP | 420 |
| MKWGEEEKIG VLVDKEGVKK AVEELMGESD DAKERRRRAK ELGDSAHKAV EEGGSSHSNI | 480 |
| SFLLQDIMEL AEPNN | 495 |

Linalool synthase (Arabidopsis thaliana) (SEQ ID NO: 8)

| | |
|---|---|
| MALIATKISS RSCFVSAYPN NSPTFLISKF PNTVDSLSPA NTAKRSILRN VHASVSNPSK | 60 |
| QFHNKTSLEY SHELNIKKIK NILSANVDVP SENLEMIDVI QSLGTDLHFR QGIEQTLHMI | 120 |
| YKEGLQFNGD LHEIALRFRL LRQEGHYVQE SIFKNILDKK GGFKDVVKND VKGLTELFEA | 180 |
| SELRVEGEET LDGAREFTYS RLNELCSGRE SHQKQEIMKS LAQPRHKTVR GLTSKRFTSM | 240 |
| IKIAGQEDPE WLQSLLRVAE IDSIRLKSLT QGEMSQTFKW WTELGLEKDV EKARSQPLKW | 300 |
| HTWSMKILQD PTLTEQRLDL TKPISLVYVI DDIFDVYGEL EELTIFTRVV ERWDHKGLKT | 360 |
| LPKYMRVCFE ALDMITTEIS MKIYKSHGWN PTYALRQSWA SLCKAFLVEA KWFNSGYLPT | 420 |
| TEEYMKNGVV SSGVHLVMLH AYILLGEELT KEKVELIESN PGIVSSAATI LRLWDDLGSA | 480 |
| KDENQDGTDG SYVECYLNEY KGSTVDEART HVAQKISRAW KRLNRECLNP CPFSRSFSKA | 540 |
| CLNIARTVPL MYSYDDDQRL PDEYLKSLM | 569 |

In addition to the genes and polypeptides contemplated in Tables 1 and 2, one of skill in the art will understand that other sequences can be used in addition to those exemplified in Tables 1 and 2. Furthermore, nucleic acid sequences encoding functional polypeptides, or the active domains (active fragments or active portions), wherein the sequences have sequence identity of at least 70%, 80%, 90%, 95%, 96%, 97%, 98%, or 99% with the polypeptide sequences listed in Table 1 can be used. The polynucleotides shown in Table 1, and those having at least approximately 70%-99% nucleic acid sequence identity to such polynucleotides, including those having at least approximately 70%, 75%, 80%, 81%, 82%, 83%, 84%, 85%, 86%, 87%, 88%, 89%, 90%, 91%, 92%, 93%, 94%, 95%, 96%, 97%, 98%, 99%, and 100% nucleic acid sequence identity to any of the sequences disclosed in the present invention, wherein the polypeptide retains the enzymatic activity, can be used. Furthermore, the genomic and non-genomic forms of the polynucleotide sequences can be used. Additionally, and preferably, plant-optimized polynucleotide sequences can be used, which are generated from the amino acid sequences shown in Table 2, for example, and, for example, such sequences can be codon optimized for expression plants, using for example, the OptimumGene™ Gene Design system (GenScript, New Jersey, US; see also (Burgess-Brown et al., 2008)).

The invention can use mutant or variant polypeptides any of whose residues may be changed from the corresponding residues shown in SEQ ID NOs:2, 4, 6, 8, 10, 12, 14, 16, 18, 20, 22, and 24 while still encoding active polypeptides, or functional fragments thereof.

In general, a variant that preserves function includes any variant in which residues at a particular position in the sequence have been substituted by other amino acids, and further includes the possibility of inserting an additional residue or residues between two residues of the parent polypeptide as well as the possibility of deleting one or more residues from the parent sequence. Preferably, the substitution is a conservative substitution (Table 3).

TABLE 3
Amino acid substitutions

| | | Preferred |
|---|---|---|
| Original residue | Exemplary substitutions | substitutions |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, Norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, Norleucine | Leu |

A “polypeptide variant” means an active polypeptide having at least about 70% amino acid sequence identity with a full-length native polypeptide sequence, and any fragment of a full-length polypeptide sequence. For example, polypeptide variants include those wherein one or more amino acid residues are added or deleted at the N- or C-terminus of the full-length native amino acid sequence. A polypeptide variant will have at least about 70% amino acid sequence identity, preferably at least about 80% amino acid sequence identity, more preferably at least about 81% amino acid sequence identity, more preferably at least about 82%-98% amino acid sequence identity and most preferably at least about 99% amino acid sequence identity with a full-length native sequence. Ordinarily, variant polypeptides are at least about 10 amino acids in length, often at least about 20 amino acids in length, more often at least about 30, 40, 50, 60, 70, 80, 90, 100, 150, 200, or 300 amino acids in length, or more.

Biologically active portions of a polypeptide are peptides comprising amino acid sequences sufficiently homologous to, or derived from, the amino acid sequences of the polypeptide that include fewer amino acids than the full-length polypeptide, and exhibit at least one activity of the full-length polypeptide. Biologically active portions (active fragments) comprise a domain or motif with at least one activity of native polypeptide. A biologically active portion of a polypeptide can be 10, 25, 50, 100 or more amino acid residues in length. Other biologically active portions, in which other regions of the polypeptide are deleted, can be prepared by recombinant techniques and evaluated for one or more of the functional activities of the native polypeptide.

Biologically active portions of a polypeptide can have an amino acid sequence shown in SEQ ID NOs:2, 4, 6, 8, 10, 12, 14, 16, 18, 20, 22, and 24 or be substantially homologous to those sequences, and retain the functional activity of the polypeptide, yet differs in amino acid sequence due to natural allelic variation or mutagenesis. Other biologically active polypeptide may comprise an amino acid sequence at least 45% homologous to the amino acid sequence of the parent polypeptide, and retains the functional activity of native polypeptide.

Vectors

Vectors are tools used to shuttle DNA between host cells or as a means to express a polynucleotide sequence, such as SEQ ID NOs:1, 3, 5, 7, 9, 11, 13, 15, 17, 19, 21, and 23. Some vectors function only in prokaryotes, while others function in both prokaryotes and eukaryotes, enabling large-scale DNA preparation from prokaryotes for expression in eukaryotes. Inserting the DNA of interest is accomplished by ligation techniques and/or mating protocols well known to the skilled artisan. Such DNA is inserted such that its integration does not disrupt any necessary components of the vector. In the case of vectors that are used to express the inserted DNA as a polypeptide, the introduced DNA is operably-linked to the vector elements that govern its transcription and translation.

Vectors can be divided into two general classes: Cloning vectors are replicating plasmid or phage with regions that are non-essential for propagation in an appropriate host cell, and into which foreign DNA can be inserted; the foreign DNA is replicated and propagated as if it were a component of the vector. An expression vector is used to introduce foreign genetic material into a host cell or tissue in order to transcribe and translate the foreign DNA. In expression vectors, the introduced DNA is operably-linked to elements, such as promoters, that signal to the host cell to transcribe the inserted DNA.

Vectors have many manifestations. A plasmid is a circular double stranded DNA molecule that can accept additional DNA fragments. Certain vectors are capable of autonomous replication in a host cell (e.g., bacterial vectors having a bacterial origin of replication). Other vectors integrate into the genome of a host cell and replicate as part of the host genome. In general, useful expression vectors are plasmids and Agrobacterium-based; other expression vectors can also be used.

In general, vectors comprise signal sequences, origins of replication, marker genes, enhancer elements, promoters, and transcription termination sequences. Vectors often use a selectable marker to facilitate identifying those cells that have incorporated the vector. Many selectable markers are well known in the art for the use with prokaryotes, usually antibiotic-resistance genes or the use of autotrophy and auxotrophy mutants, as are those selectable markers for use with plant material. Other screenable markers may be used.

“Host cell” and “recombinant host cell” are used interchangeably. Such terms refer not only to a particular subject cell but also to the progeny or potential progeny of such a cell. Because certain modifications may occur in succeeding generations due to either mutation or environmental influences, such progeny may not, in fact, be identical to the parent cell, but are still included within the scope of the term.

Some promoters are exceptionally useful, such as inducible promoters that control gene transcription in response to specific factors. Similarly, tissue-specific promoters relegate expression to specific tissues in the plant. Operably-linking a transgene to an inducible or tissue-specific promoter can control the expression of the expressed molecule. Exemplary classes of plant promoters are described below.

Exemplary constitutive expression promoters include the ubiquitin promoter, the CaMV 35S promoter (U.S. Pat. Nos. 5,858,742 and 5,322,938); and the actin promoter (e.g., rice; U.S. Pat. No. 5,641,876).

Exemplary inducible expression promoters include the chemically regulatable tobacco PR-1 promoter (e.g., tobacco, U.S. Pat. No. 5,614,395; maize, U.S. Pat. No. 6,429,362). Various chemical regulators can be used to induce expression, including the benzothiadiazole, isonicotinic acid, and salicylic acid compounds disclosed in U.S. Pat. Nos. 5,523,311 and 5,614,395. Other promoters inducible by certain alcohols or ketones, such as ethanol, include the alcA gene promoter from Aspergillus nidulans. Glucocorticoid-mediated induction systems can also be used (Aoyama and Chua, 1997). Another class of useful promoters are water-deficit-inducible promoters, e.g., promoters that are derived from the 5′ regulatory region of genes identified as a heat shock protein 17.5 gene (HSP 17.5), an HVA22 gene (HVA22), and a cinnamic acid 4-hydroxylase gene (CA4H) of Zea mays. Another water-deficit-inducible promoter is derived from the rab-17 promoter. U.S. Pat. No. 6,084,089 discloses cold inducible promoters, U.S. Pat. No. 6,294,714 discloses light inducible promoters, U.S. Pat. No. 6,140,078 discloses salt inducible promoters, U.S. Pat. No. 6,252,138 discloses pathogen inducible promoters, and U.S. Pat. No. 6,175,060 discloses phosphorus deficiency inducible promoters. Wound-inducible promoters can also be used.

Exemplary promoters that express genes only in certain tissues (tissue-specific promoters) are useful. For example, root-specific expression can be attained using the promoter of the maize metallothionein-like (MTL) gene (U.S. Pat. No. 5,466,785). U.S. Pat. No. 5,837,848 discloses a root-specific promoter. Another exemplary promoter confers pith-preferred expression (maize trpA gene and promoter; WO 93/07278). Leaf-specific expression can be attained, for example, by using the promoter for a maize gene encoding phosphoenol carboxylase. Pollen-specific expression can be conferred by the promoter for the maize calcium-dependent protein kinase (CDPK) gene that is expressed in pollen cells (WO 93/07278). U.S. Pat. Appl. Pub. No. 20040016025 describes tissue-specific promoters. Pollen-specific expression can also be conferred by the tomato LAT52 pollen-specific promoter. U.S. Pat. No. 6,437,217 discloses a root-specific maize RS81 promoter, U.S. Pat. No. 6,426,446 discloses a root specific maize RS324 promoter, U.S. Pat. No. 6,232,526 discloses a constitutive maize A3 promoter, U.S. Pat. No. 6,177,611 that discloses constitutive maize promoters, U.S. Pat. No. 6,433,252 discloses a maize L3 oleosin promoter that is aleurone and seed coat-specific, U.S. Pat. No. 6,429,357 discloses a constitutive rice actin 2 promoter and intron, U.S. patent application Pub. No. 20040216189 discloses an inducible constitutive leaf-specific maize chloroplast aldolase promoter.

Optionally a plant transcriptional terminator can be used in place of the plant-expressed gene native transcriptional terminator. Exemplary transcriptional terminators are those that are known to function in plants and include the CaMV 35S terminator, the tml terminator, the nopaline synthase terminator and the pea rbcS E9 terminator. These can be used in both monocotyledons and dicotyledons.

Various intron sequences have been shown to enhance expression. For example, the introns of the maize Adh1 gene can significantly enhance expression, especially intron 1 (Callis et al., 1987). The intron from the maize bronzel gene also enhances expression. Intron sequences have been routinely incorporated into plant transformation vectors, typically within the non-translated leader. U.S. Patent Application Publication 2002/0192813 discloses 5′, 3′ and intron elements useful in the design of effective plant expression vectors.

A number of non-translated leader sequences derived from viruses are also known to enhance expression, and these are particularly effective in dicotyledonous cells. Specifically, leader sequences from Tobacco Mosaic Virus (TMV, the “omega-sequence”), Maize Chlorotic Mottle Virus (MCMV), and Alfalfa Mosaic Virus (AMV) can enhance expression. Other leader sequences known and include: picornavirus leaders, for example, EMCV leader (Encephalomyocarditis 5′ noncoding region); potyvirus leaders, for example, TEV leader (Tobacco Etch Virus); MDMV leader (Maize Dwarf Mosaic Virus); human immunoglobulin heavy-chain binding protein (BiP) leader; untranslated leader from the coat protein mRNA of AMV (AMV RNA 4); TMV virus leader; or MCMV leader (MCMV).

A minimal promoter can also be incorporated. Such a promoter has low background activity in plants when there is no transactivator present or when enhancer or response element binding sites are absent. An example is the Bzl minimal promoter, obtained from the bronze/gene of maize. A minimal promoter can also be created by use of a synthetic TATA element. The TATA element allows recognition of the promoter by RNA polymerase factors and confers a basal level of gene expression in the absence of activation.

Sequences controlling the targeting of gene products also can be included. For example, the targeting of gene products to the chloroplast is controlled by a signal sequence found at the amino terminal end of various proteins that is cleaved during chloroplast import to yield the mature protein. These signal sequences can be fused to heterologous gene products to import heterologous products into the chloroplast. DNA encoding for appropriate signal sequences can be isolated from the 5′ end of the cDNAs encoding the RUBISCO protein, the CAB protein, the EPSP synthase enzyme, the GS2 protein or many other proteins that are known to be chloroplast localized. Other gene products are localized to other organelles, such as the mitochondrion and the peroxisome (e.g., (Unger et al., 1989)). Examples of sequences that target to such organelles are the nuclear-encoded ATPases or specific aspartate amino transferase isoforms for mitochondria. Amino terminal and carboxy-terminal sequences are responsible for targeting to the ER, the apoplast, and extracellular secretion from aleurone cells. Amino terminal sequences in conjunction with carboxy terminal sequences can target to the vacuole.

Another element that can be introduced is a matrix attachment region element (MAR), such as the chicken lysozyme A element that can be positioned around an expressible gene of interest to effect an increase in overall expression of the gene and diminish position dependent effects upon incorporation into the plant genome.

The promoter can be derived from plant or non-plant species. For example, the nucleotide sequence of the promoter is derived from non-plant species for the expression of genes in plant cells, such as dicotyledonous plant cells, such as cotton. Non-plant promoters can be constitutive or inducible promoters derived from insects, e.g., Drosophila melanogaster, or from yeast, e.g., Saccharomyces cerevisiae. Other desirable inducible promoters include those that are not endogenous to the cells in which the construct is being introduced, but, however, are responsive in those cells when the induction agent is exogenously supplied.

Genetic Transformation

In some embodiments, DNA constructs are used to introduce genes into the genome of a plant cell or plant and are exploited to express the target transgenes of interest. Any plant, including bryophytes, algae, seedless vascular plants, monocots, dicots, gymnosperm, field crops, vegetable crops, fruit and vine crops, can be modified. Plant parts or plant tissues, including pollen, silk, endosperm, ovule, seed, embryo, pods, roots, cuttings, tubers, stems, stalks, fiber (lint), square, boll, fruit, berries, nuts, flowers, leaves, bark, epidermis, vascular tissue, whole plant, plant cell, plant organ, protoplast, crown, callus culture, petiole, petal, sepal, stamen, stigma, style, bud, meristem, cambium, cortex, pith, sheath, cell culture, or any group of plant cells organized into a structural and functional unit, any cells of plants can carry recombinant constructs.

A related aspect of the invention is plant parts or plant tissues, including pollen, silk, endosperm, ovule, seed, embryo, pods, roots, cuttings, tubers, stems, stalks, crown, fiber (lint), square, boll, callus culture, petiole, petal, sepal, stamen, stigma, style, bud, fruit, berries, nuts, flowers, leaves, bark, wood, whole plant, plant cell, plant organ, protoplast, cell culture, or any group of plant cells organized into a structural and functional unit comprising the nucleic acid of interest, whether maintained autonomously (such as on a plant artificial chromosome, such as a mini-chromosome) or integrated into the host plant cell chromosomes. In one preferred embodiment, the exogenous nucleic acid is primarily expressed in a specific location or tissue of a plant, for example, epidermis, fiber (lint), boll, square, vascular tissue, meristem, cambium, cortex, pith, leaf, sheath, flower, root or seed. Tissue-specific expression can be accomplished with, for example, promoters that drive tissue-specific expression.

Suitable methods include any method by which DNA can be introduced into a cell, such as by Agrobacterium or viral infection, direct delivery of DNA such as, for example, by PEG-mediated transformation of protoplasts (Omirulleh et al., 1993), by desiccation/inhibition-mediated DNA uptake, by electroporation, by agitation with silicon carbide fibers, by acceleration of DNA coated particles, etc. In certain embodiments, acceleration methods are preferred and include, for example, microprojectile bombardment.

Technology for introduction of DNA into cells is well-known to those of skill in the art. Four general methods for delivering a gene into cells have been described: (1) chemical methods (Graham and van der Eb, 1973; Zatloukal et al., 1992); (2) physical methods such as microinjection (Capecchi, 1980), electroporation (Fromm et al., 1985; Wong and Neumann, 1982) and the gene gun (Fynan et al., 1993; Johnston and Tang, 1994); (3) viral vectors (Clapp, 1993; Eglitis and Anderson, 1988; Eglitis et al., 1988; Lu et al., 1993); and (4) receptor-mediated mechanisms (Curiel et al., 1991; Curiel et al., 1992; Wagner et al., 1992).

Electroporation can be extremely efficient and can be used both for transient expression of cloned genes and for establishment of cell lines that carry integrated copies of the gene of interest. The introduction of DNA by electroporation is well-known to those of skill in the art. In this method, certain cell wall-degrading enzymes, such as pectin-degrading enzymes, are employed to render the target recipient cells more susceptible to transformation by electroporation than untreated cells. Alternatively, recipient cells are made susceptible to transformation by mechanical wounding. To effect transformation by electroporation, one can use either friable tissues, such as a suspension culture of cells or embryogenic callus, or alternatively one can transform immature embryos or other organized tissues directly.

Microprojectile bombardment shoots particles coated with the DNA of interest into to plant cells. Exemplary particles include tungsten, gold, and platinum, preferably 1 micron gold particles. Specialized biolistics devices, such as are available from Bio-Rad Laboratories (Hercules, Calif.; US) can be used. An advantage of microprojectile bombardment is that protoplast isolation is unnecessary, and a requirement for susceptibility to Agrobacterium infection is not required. For bombardment, cells in suspension are preferably concentrated on filters or solid culture medium. Alternatively, immature embryos or other target cells can be arranged on solid culture medium. The cells are positioned below a macroprojectile stopping plate. If desired, one or more screens are also positioned between the acceleration device and the cells to be bombarded.

Various biolistics protocols have been described that differ in the type of particle or the manner in that DNA is coated onto the particle. Any technique for coating microprojectiles that allows for delivery of transforming DNA to the target cells can be used. For example, particles can be prepared by functionalizing the surface of a gold oxide particle by providing free amine groups. DNA, having a strong negative charge, binds to the functionalized particles.

Parameters such as the concentration of DNA used to coat microprojectiles can influence the recovery of transformants containing a single copy of the transgene. For example, a lower concentration of DNA may not necessarily change the efficiency of the transformation but can instead increase the proportion of single copy insertion events. Ranges of approximately 1 ng to approximately 10 pg, approximately 5 ng to 8 μg or approximately 20 ng, 50 ng, 100 ng, 200 ng, 500 ng, 1 pg, 2 μg, 5 μg, or 7 μg of transforming DNA can be used per each 1.0-2.0 mg of starting 1.0 micron gold particles.

Other physical and biological parameters can be varied, such as manipulation of the DNA/microprojectile precipitate, factors that affect the flight and velocity of the projectiles, manipulation of the cells before and immediately after bombardment (including osmotic state, tissue hydration and the subculture stage or cell cycle of the recipient cells), the orientation of an immature embryo or other target tissue relative to the particle trajectory, and also the nature of the transforming DNA, such as linearized DNA or intact supercoiled plasmids. Physical parameters such as DNA concentration, gap distance, flight distance, tissue distance, and helium pressure, can be optimized.

The particles delivered via biolistics can be “dry” or “wet.” In the “dry” method, the DNA-coated particles such as gold are applied onto a macrocarrier (such as a metal plate, or a carrier sheet made of a fragile material, such as mylar) and dried. The gas discharge then accelerates the macrocarrier into a stopping screen that halts the macrocarrier but allows the particles to pass through. The particles are accelerated at, and enter, the plant tissue arrayed below on growth media. The media supports plant tissue growth and development and are suitable for plant transformation and regeneration. These tissue culture media can either be purchased as a commercial preparation, or custom prepared and modified. Examples of such media include Murashige and Skoog (MS), N6, Linsmaier and Skoog, Uchimiya and Murashige, Gamborg's B5 media, D medium, McCown's Woody plant media, Nitsch and Nitsch, and Schenk and Hildebrandt. Those of skill in the art are aware that media and media supplements such as nutrients and growth regulators for use in transformation and regeneration and other culture conditions such as light intensity during incubation, pH, and incubation temperatures can be optimized.

Those of skill in the art can use, devise, and modify selective regimes, media, and growth conditions depending on the plant system and the selective agent. Typical selective agents include antibiotics, such as GENETICIN® (G418), kanamycin, paromomycin; or other chemicals, such as glyphosate or other herbicides.

Agrobacterium-mediated transfer is a widely applicable system for introducing genes into plant cells because the DNA can be introduced into whole plant tissues, thereby bypassing the need for regeneration of an intact plant from a protoplast. Dafny-Yelin et al. provide an overview of Agrobacterium transformation (Dafny-Yelin and Tzfira, 2007). Agrobacterium plant integrating vectors to introduce DNA into plant cells is well known in the art, such as those described above, as well as others (Rogers et al., 1987). Further, the integration of the Ti-DNA is a relatively precise process resulting in few rearrangements. The region of DNA to be transferred is defined by the border sequences (Jorgensen et al., 1987; Spielmann and Simpson, 1986).

A transgenic plant formed using Agrobacterium transformation methods typically contains a single gene on one chromosome. Homozygous transgenic plants can be obtained by sexually mating (selfing) an independent segregant transgenic plant that contains a single added gene, germinating some of the seed produced and analyzing the resulting plants for the targeted trait or insertion.

Several Agrobacterium species mediate the transfer of T-DNA that can be genetically engineered to carry a desired piece of DNA into many plant species. Plasmids used for delivery contain the T-DNA flanking the nucleic acid to be inserted into the plant. The major events marking the process of T-DNA mediated pathogenesis are induction of virulence genes, processing and transfer of T-DNA.

There are three common methods to transform plant cells with Agrobacterium. The first method is co-cultivation of Agrobacterium with cultured isolated protoplasts. This method requires an established culture system that allows culturing protoplasts and plant regeneration from cultured protoplasts. The second method is transformation of cells or tissues with Agrobacterium. This method requires (1) that the plant cells or tissues can be modified by Agrobacterium and (2) that the modified cells or tissues can be induced to regenerate into whole plants. The third method is transformation of seeds, apices or meristems with Agrobacterium. This method requires exposure of the meristematic cells of these tissues to Agrobacterium and micropropagation of the shoots or plant organs arising from these meristematic cells.

Those of skill in the art are familiar with procedures for growth and suitable culture conditions for Agrobacterium, as well as subsequent inoculation procedures. Liquid or semi-solid culture media can be used. The density of the Agrobacterium culture used for inoculation and the ratio of Agrobacterium cells to explant can vary from one system to the next, as can media, growth procedures, timing and lighting conditions.

Transformation of dicotyledons using Agrobacterium has long been known in the art, and transformation of monocotyledons using Agrobacterium has also been described (WO 94/00977; U.S. Pat. No. 5,591,616; US20040244075).

A number of wild-type and disarmed strains of Agrobacterium tumefaciens and Agrobacterium rhizogenes harboring Ti or Ri plasmids can be used for gene transfer into plants. Preferably, the Agrobacterium hosts contain disarmed Ti and Ri plasmids that do not contain the oncogenes that cause tumorigenesis or rhizogenesis. Exemplary strains include Agrobacterium tumefaciens strain CSS, a nopaline-type strain that is used to mediate the transfer of DNA into a plant cell, octopine-type strains such as LBA4404, or succinamopine-type strains, e.g., EHA101 or EHA105.

The efficiency of transformation by Agrobacterium can be enhanced by using a number of methods known in the art. For example, the inclusion of a natural wound response molecule such as acetosyringone (AS) to the Agrobacterium culture can enhance transformation efficiency with Agrobacterium tumefaciens. Alternatively, transformation efficiency can be enhanced by wounding the target tissue to be modified or transformed. Wounding of plant tissue can be achieved, for example, by punching, maceration, bombardment with microprojectiles, etc.

Transformation with Selectable Marker Gene

Transgene-modified cells in bombarded calluses or explants can be isolated using a selectable marker gene. The bombarded tissues are transferred to a medium containing an appropriate selective agent. Tissues are transferred into selection between 0 and about 7 days or more after bombardment. Selection of transgene-modified cells can be further monitored by tracking screenable markers, such as fluorescent marker genes, or by the appearance of modified explants (modified cells on explants can be green under light in selection medium, while surrounding non-modified cells are weakly pigmented). In plants that develop through shoot organogenesis (e.g., Brassica, tomato or tobacco), the modified cells can form shoots directly, or alternatively, can be isolated and expanded for regeneration of multiple transgenic shoots. In plants that develop through embryogenesis (e.g., corn or soybean), additional culturing steps may be necessary to induce the modified cells to form an embryo and to regenerate in the appropriate media.

For selection to be effective, the plant cells or tissue need to be grown on selective medium containing the appropriate concentration of antibiotic or killing agent, and the cells need to be plated at a defined and constant density. The concentration of selective agent and cell density are generally chosen to cause complete growth inhibition of wild type plant tissue that does not express the selectable marker gene; but allowing cells containing the introduced DNA to grow and expand into clones. This critical concentration of selective agent typically is the lowest concentration at that there is complete growth inhibition of wild type cells, at the cell density used in the experiments.

Regeneration of Transgenic Plants from Explants to Mature, Rooted Plants

For plants that develop through shoot organogenesis (e.g., Brassica, tomato and tobacco), regeneration of a whole plant involves culturing of regenerable explant tissues taken from sterile organogenic callus tissue, seedlings or mature plants on a shoot regeneration medium for shoot organogenesis, and rooting of the regenerated shoots in a rooting medium to obtain intact whole plants with a fully developed root system.

For some plant species, such as cotton, corn, and soybean, regeneration of a whole plant occurs via an embryogenic step that is not necessary for plant species where shoot organogenesis is efficient. In these plants, the explant tissue is cultured on an appropriate media for embryogenesis, and the embryo is cultured until shoots form. The regenerated shoots are cultured in a rooting medium to obtain intact whole plants with a fully developed root system.

Explants are obtained from any tissues of a plant suitable for regeneration. Exemplary tissues include hypocotyls, internodes, roots, cotyledons, petioles, cotyledonary petioles, leaves and peduncles, prepared from sterile seedlings or mature plants.

Explants are wounded (for example with a scalpel or razor blade) and cultured on a shoot regeneration medium (SRM) containing MS medium as well as a cytokinin, e.g., 6-benzylaminopurinc (BA), and an auxin, e.g., α-naphthaleneacetic acid (NAA), and an anti-ethylene agent, e.g., silver nitrate (AgNO₃). For example, 2 mg/L of BA, 0.05 mg/L of NAA, and 2 mg/L of AgNO₃ can be added to MS medium for shoot organogenesis. The most efficient shoot regeneration is often obtained from longitudinal sections of internode explants.

Shoots regenerated via organogenesis are rooted in a MS medium containing low concentrations of an auxin, such as NAA.

To regenerate a whole plant with the transgene of interest, explants are pre-incubated for 1 to 7 days (or longer) on the shoot regeneration medium prior to bombardment. Following bombardment, explants are incubated on the same shoot regeneration medium for a recovery period up to 7 days (or longer), followed by selection for transformed shoots or clusters on the same medium but with a selective agent appropriate for a particular selectable marker gene.

Analyses of Transformed Plants

Determination of Gene Expression Levels

The expression level of any gene present in the transformant can be determined by several methods, such as for RNA, Northern Blot hybridization, Reverse Transcriptase-PCR, binding levels of a specific RNA-binding protein, in situ hybridization, or dot blot hybridization; or for proteins, Western blot hybridization, Enzyme-Linked Immunosorbant Assay (ELISA), fluorescent quantitation of a fluorescent gene product, enzymatic quantitation of an enzymatic gene product, immunohistochemical quantitation, or spectroscopic quantitation of a gene product that absorbs a specific wavelength of light.

Processing of Transgenic Plants for Terpenoid Biofuel

Farnesene has been extracted from plant tissues using solid-phase microextraction (SPME) (Demyttenaere et al., 2004; Zini et al., 2003), subcritical CO₂ extraction (Rout et al., 2008), microwave-assisted solvent extraction (Serrano and Gallego, 2006), and two-stage solvent extraction (Pechous et al., 2005). Ionic liquid methods to extract aromatic and aliphatic hydrocarbons (Arce et al., 2008; Arce et al., 2007) can also be used for farnesene extraction. While chipped and ground dry plants, sometimes coupled with pelletization, have been effectively extracted using solvents, further disruption or poration of plant cell walls can increase extraction efficiency. The effect of various low cost pretreatment methods can be tested, including mild alkali or acid treatment, ammonia explosion, and steam explosion on extraction efficiency and product purity. Ultrasound-assisted extraction (Hernanz et al., 2008), liquid-liquid extraction at high pressure, and/or high temperature also can assist in solvent penetration (into the cell wall) and improve farnesene extraction.

The present invention permits farnesol harvest by chopping or grinding the transgenic plant material that has accumulated farnesol or farnesol glucosides, and in the case of farnesol glucosides, having endogenous glycosidases act on the farnesol glucosides to yield farnesol. In other embodiments, glycosidases are added to the material, either before, during, or after chopping or grinding. Examples of glycosidases include: arabinases, fucosidases, galactosidases, galactanases, arabico-galactan-galactosidases, mannanases (also called mannosidases or mannases), glucuronosidases, agarase, carrageenases, pullulanases, xyloglucanases (xylanases), xanthanases, and pectin-degrading enzymes (pectinases).

Field Trials with Transgenic Plants

Transgenic plant cell lines are regenerated, proliferated (to make genetically-identical replicates of each transgenic line), rooted, acclimated and used in field trials. For seed-bearing plants, seed is collected and segregated.

Descriptor data from typical plants of each transgenic accession plus tissue-cultured and regenerated from wild type and empty vector lines is collected at regular intervals over at least a year or more, depending on the type of plant transformed and is easily determined by one of skill in the art. Descriptors for which data can be collected include:
a. Morphological: flower color and size, seed size and weight, leaf color, leaf size, leaf margin teeth, number of branches from the main stem.
b. Growth: plant height and width, fresh and dry weight.
c. Chemical: farnesol, total resin, and total hydrocarbon content.
d. Phenology: first flower date, 50% bloom date, and seed maturity date (first seed harvest).
e. Seed production: total seed mass and weight
f. Imaging: digital images of entire plants, and of the leaves, flowers and seeds.
   Descriptor data (morphological, chemical, phonological, growth, production, and imaging) are collected, descriptive statistics performed and results analyzed. Seeds from selected transgenic lines that approach or meet a predetermined target are further propagated for large scale field trials. In this experiment, secondary input targets such as water requirements fertilizer requirement, and management practices are typically evaluated.

NIR can be used to follow farnesol accumulation during the growing season. Plants from the field trials can also provide the materials needed for the initial extraction scale-up. Experiments can also be conducted to determine the stability of farnesol post-harvest in whole, chopped and chipped plants, and under a range of storage conditions varying time, temperature and humidity.

Channeling Carbon Flux Toward Sesquiterpene Synthesis Using PGRs and Herbicides

In some embodiments of the invention, carbon flux is directed toward sesquiterpene production (and away from carotenoid and sterol synthesis) using PGRs and herbicides. The PGRs and herbicides can be used preferably on the modified plants of the present invention (having increased production of farnesene and/or farnesol), or on un-modified plants.

In one embodiment, ethylene stimulators of ethylene synthesis are used, such as ethephon, etacelasil, glyoxime, ACC, or others. Inhibitors of phytoene synthase, such as gerfelin or bisphosphonate analogs of geranylpyrophosphate can be used, as well as squalene epoxidase inhibitors of sterol biosynthesis (such as monooxygenase inhibitor-based herbicides in the allylamine and thiocarbamate classes, such as pyributicar. Finally, compounds that engender systemic acquired resistance can be used, such as benzo (1,2,3) thiadiazole-7-carbothioic acid S-methyl ester (BION®) and salicylic acid.

The modes of application, rates of application, and the timing (before cutting, prior to harvest, post harvest, or all) can readily be determined by one of skill in the art.

### DEFINITIONS

“Consisting essentially of a polynucleotide having a % sequence identity” means that the polynucleotide does not substantially differ in length, but may differ substantially in sequence. Thus, a polynucleotide “A” consisting essentially of a polynucleotide having at least 80% sequence identity to a known sequence “B” of 100 nucleotides means that polynucleotide “A” is about 100 nts long, but up to 20 nts can vary from the “B” sequence. The polynucleotide sequence in question can be longer or shorter due to modification of the termini, such as, for example, the addition of 1-15 nucleotides to produce specific types of probes, primers and other molecular tools, etc., such as the case of when substantially non-identical sequences are added to create intended secondary structures. Such non-identical nucleotides are not considered in the calculation of sequence identity when the sequence is modified by “consisting essentially of.”

The specificity of single stranded DNA to hybridize complementary fragments is determined by the stringency of the reaction conditions. Hybridization stringency increases as the propensity to form DNA duplexes decreases. In nucleic acid hybridization reactions, the stringency can be chosen to favor specific hybridizations (high stringency). Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide, which decrease DNA duplex stability. A common approach is to vary the temperature: higher relative temperatures result in more stringent reaction conditions. (Ausubel et al., 1987) provide an excellent explanation of stringency of hybridization reactions.

“Constitutive active promoter” means a promoter that allows permanent and stable expression of the gene of interest.

“Exogenous” when used in reference to a nucleic acid, for example, refers to any nucleic acid that has been introduced into a recipient cell, regardless of whether the same or similar nucleic acid is already present in such a cell. An “exogenous gene” can be a gene not normally found in the host genome in an identical context, or an extra copy of a host gene. The gene can be isolated from a different species than that of the host genome, or alternatively, isolated from the host genome but operably linked to one or more regulatory regions that differ from those found in the unaltered, native gene. The gene can also be synthesized in vitro.

“Farnesene” means a 15 carbon class of closely related chemical compounds, all of which are sesquiterpenes. α-farnesene (3,7,11-trimethyl-1,3,6,10-dodecatetraene) has the formula (I):

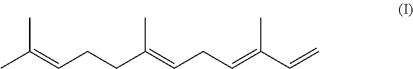

α-farnesene can exist as four isomers. β-farnesene (7,11-dimethyl-3-methylene-1,6,10-dodecatriene) has the formula (II):

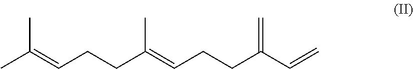

β-farnesene can exist as two isomers.

“Farnesol” means a 15 carbon acyclic sesquiterpene alcohol, (2E,6E)-3,7,11-trimethyldodeca-2,6,10-trien-1-ol, and having the formula (III):

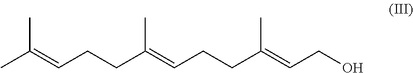 [SMILES: CC(C)=CCC/C(C)=C/CC/C(C)=C/CO]

“Guayule” means the desert shrub, Parthenium argentatum, native to the southwestern United States and northern Mexico and which produces polymeric isoprene essentially identical to that made by Hevea rubber trees (e.g., Hevea brasiliensis) in Southeast Asia.

“Hybridizes under low stringency, medium stringency, and high stringency conditions” describes conditions for hybridization and washing. Hybridization is a well-known technique (Ausubel, 1987). Low stringency hybridization conditions means, for example, hybridization in 6× sodium chloride/sodium citrate (SSC) at about 45° C., followed by two washes in 0.5×SSC, 0.1% SDS, at least at 50° C.; medium stringency hybridization conditions means, for example, hybridization in 6×SSC at about 45° C., followed by one or more washes in 0.2×SSC, 0.1%) SDS at 55° C.; and high stringency hybridization conditions means, for example, hybridization in 6×SSC at about 45° C., followed by one or more washes in 0.2×SSC, 0.1% SDS at 65° C. Another non limiting example of stringent hybridization conditions are hybridization in a high salt buffer comprising 6×SSC, 50 mM Tris HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. Another non limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Another non limiting example of low stringency hybridization conditions are hybridization in 35% formamide, 5×SSC, 50 mM Tris HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris HCl (pH 7.4), 5 mM EDTA, and 0.1% SDS at 50° C. Other conditions of low stringency that may be used are well known in the art (e.g., as employed for cross species hybridizations).

Polynucleotides can include other appended groups such as peptides (e.g., for targeting host cell receptors in vivo), or agents facilitating transport across the cell membrane. In addition, oligonucleotides can be modified with hybridization-triggered cleavage agents (van der Krol et al., 1988) or intercalating agents (Zon, 1988). The oligonucleotide can be conjugated to another molecule, e.g., a peptide, a hybridization triggered cross-linking agent, a transport agent, a hybridization-triggered cleavage agent, and the like.

Useful polynucleotide analogues include polymers having modified backbones or non-natural inter-nucleoside linkages. Modified backbones include those retaining a phosphorus atom in the backbone, such as phosphorothioates, chiral phosphorothioates, phosphorodithioates, phosphotriesters, aminoalkylphosphotriesters, methyl and other alkyl phosphonates, as well as those no longer having a phosphorus atom, such as backbones formed by short chain alkyl or cycloalkyl inter-nucleoside linkages, mixed heteroatom and alkyl or cycloalkyl inter-nucleoside linkages, or one or more short chain heteroatomic or heterocyclic inter-nucleoside linkages. Modified nucleic acid polymers (analogues) can contain one or more modified sugar moieties.

Analogs that are RNA or DNA mimetics, in which both the sugar and the inter-nucleoside linkage of the nucleotide units are replaced with novel groups, are also useful. In these mimetics, the base units are maintained for hybridization with the target sequence. An example of such a mimetic, which has been shown to have excellent hybridization properties, is a peptide nucleic acid (PNA) (Buchardt et al., 1992; Petrasovits, 2007).

The realm of nucleotides includes derivatives wherein the nucleic acid molecule has been covalently modified by substitution, chemical, enzymatic, or other appropriate means with a moiety other than a naturally occurring nucleotide.

The polynucleotides disclosed in the present invention can be prepared by conventional techniques, such as solid-phase synthesis using commercially available equipment, such as that available from Applied Biosystems USA Inc. (Foster City, Calif.; USA), DuPont, (Wilmington, Del.; USA), or Milligen (Bedford, Mass.; USA). Modified polynucleotides, such as phosphorothioates and alkylated derivatives, can also be readily prepared by similar methods known in the art (Fino, 1995; Mattingly, 1995; Ruth, 1990).

“Inducible promoter” means a promoter induced by the presence or absence of a biotic or an abiotic factor.

“Operably linked” is defined as a configuration in that a control sequence, e.g., a promoter sequence, directs transcription or translation of another sequence, for example a coding sequence. For example, a promoter sequence could be appropriately placed at a position relative to a coding sequence such that the control sequence directs the production of a polypeptide encoded by the coding sequence.

“Percent (%) amino acid sequence identity” is defined as the percentage of amino acid residues that are identical with amino acid residues in a reference sequence in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Publicly available computer software such as BLAST, BLAST2, ALIGN2 or Megalign (DNASTAR) can be used to align polypeptide sequences. Those skilled in the art will determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:

% amino acid sequence identity=X/Y·100

where

X is the number of amino acid residues scored as identical matches by the sequence alignment program's or algorithm's alignment of A and B

and

Y is the total number of amino acid residues in B.

If the length of amino acid sequence A is not equal to the length of amino acid sequence B, the % amino acid sequence identity of A to B will not equal the % amino acid sequence identity of B to A.

“Percent (%) nucleic acid sequence identity” can be obtained by the comparison of sequences and determination of percent identity between two nucleotide sequences can be accomplished using a mathematical algorithm. For example, the percent identity between two amino acid sequences can be determined using the Needleman and Wunsch algorithm that has been incorporated into the GAP program in the GCG software package (Needleman and Wunsch, 1970), using either a Blossum 62 matrix or a PAM250 matrix. Parameters are set so as to maximize the percent identity. As further exemplification, and with respect to nucleic acid sequences is defined as the percentage of nucleotides in a candidate sequence that are identical with the nucleotides in the sequence of interest, after aligning the sequences and introducing gaps, if necessary, to achieve the maximum percent sequence identity. Alignment for purposes of determining % nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

When nucleotide sequences are aligned, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) can be calculated as follows:

% nucleic acid sequence identity=W/Z·100

where

W is the number of nucleotides cored as identical matches by the sequence alignment program's or algorithm's alignment of C and D

and

Z is the total number of nucleotides in D.

When the length of nucleic acid sequence C is not equal to the length of nucleic acid sequence D, the % nucleic acid sequence identity of C to D will not equal the % nucleic acid sequence identity of D to C.

The term “plant,” as used herein, refers to any type of plant. Exemplary types of plants are listed below, but other types of plants will be known to those of skill in the art and could be used with the invention. Modified plants of the invention include, for example, dicots, gymnosperm, monocots, mosses, ferns, horsetails, club mosses, liver worts, homworts, red algae, brown algae, gametophytes and sporophytes of pteridophytes, and green algae.

A common class of plants exploited in agriculture are vegetable crops, including artichokes, kohlrabi, arugula, leeks, asparagus, lettuce (e.g., head, leaf, romaine), bok choy, malanga, broccoli, melons (e.g., muskmelon, watermelon, crenshaw, honeydew, cantaloupe), brussels sprouts, cabbage, cardoni, carrots, napa, cauliflower, okra, onions, celery, parsley, chick peas, parsnips, chicory, Chinese cabbage, peppers, collards, potatoes, cucumber plants (marrows, cucumbers), pumpkins, cucurbits, radishes, dry bulb onions, rutabaga, eggplant, salsify, escarole, shallots, endive, garlic, spinach, green onions, squash, greens, beet (sugar beet or fodder beet), sweet potatoes, swiss chard, horseradish, tomatoes, kale, turnips, or spices.

Other types of plants frequently finding commercial use include fruit and vine crops such as apples, grapes, apricots, cherries, nectarines, peaches, pears, plums, prunes, quince, almonds, chestnuts, filberts, pecans, pistachios, walnuts, citrus, blueberries, boysenberries, cranberries, currants, loganberries, raspberries, strawberries, blackberries, grapes, avocados, bananas, kiwi, persimmons, pomegranate, pineapple, tropical fruits, pomes, melon, mango, papaya, or lychee.

Modified wood and fiber or pulp plants of particular interest include, but are not limited to maple, oak, cherry, mahogany, poplar, aspen, birch, beech, spruce, fir, kenaf, pine, walnut, cedar, redwood, chestnut, acacia, bombax, alder, eucalyptus, catalpa, mulberry, persimmon, ash, honeylocust, sweetgum, privet, sycamore, magnolia, sourwood, cottonwood, mesquite, buckthorn, locust, willow, elderberry, teak, linden, bubinga, basswood or elm.

Modified flowers and ornamental plants of particular interest, include roses, petunias, pansy, peony, olive, begonias, violets, phlox, nasturtiums, irises, lilies, orchids, vinca, philodendron, poinsettias, opuntia, cyclamen, magnolia, dogwood, azalea, redbud, boxwood, Viburnum, maple, elderberry, hosta, agave, asters, sunflower, pansies, hibiscus, morning glory, alstromeria, zinnia, geranium, Prosopis, artemesia, clematis, delphinium, dianthus, gallium, coreopsis, iberis, lamium, poppy, lavender, leucophyllum, sedum, salvia, verbascum, digitalis, penstemon, savory, pythrethrum, or oenolhera. Modified nut-bearing trees of particular interest include, but are not limited to pecans, walnuts, macadamia nuts, hazelnuts, almonds, or pistachios, cashews, pignolas or chestnuts.

Many of the most widely grown plants are field crop plants such as evening primrose, meadow foam, corn (field, sweet, popcorn), hops, jojoba, peanuts, rice, safflower, small grains (barley, oats, rye, wheat, etc.), sorghum, tobacco, kapok, leguminous plants (beans, lentils, peas, soybeans), oil plants (rape, mustard, poppy, olives, sunflowers, coconut, castor oil plants, cocoa beans, groundnuts, oil palms), fibre plants (cotton, flax, hemp, jute), lauraceae (cinnamon, camphor), or plants such as coffee, sugarcane, cocoa, tea, or natural rubber plants.

Still other examples of plants include bedding plants such as flowers, cactus, succulents or ornamental plants, as well as trees such as forest (broad-leaved trees or evergreens, such as conifers), fruit, ornamental, or nut-bearing trees, as well as shrubs or other nursery stock.

Modified crop plants include soybean (Glycine max), cotton, canola (also known as rape), wheat, sunflower, sorghum, alfalfa, barley, safflower, millet, rice, tobacco, fruit and vegetable crops or turfgrasses. Exemplary cereals include maize, wheat, barley, oats, rye, millet, sorghum, rice triticale, secale, einkorn, spelt, emmer, teff, milo, flax, gramma grass, Tripsacum sp., or teosinte. Oil-producing plants include plant species that produce and store triacylglycerol in specific organs, primarily in seeds. Such species include soybean (Glycine max), rapeseed or canola (including Brassica napus, Brassica rapa or Brassica campestris), Brassica juncea, Brassica carinata, sunflower (Helianthus annuus), cotton (including Gossypium hirsutum), com (Zea mays), cocoa (Theobroma cacao), safflower (Carthamus tinctorius), oil palm (Elaeis guineensis), coconut palm (Cocos nucifera), flax {Linum usitatissimum), castor (Ricinus communis) or peanut (Arachis hypogaea).

“Plant part” includes pollen, silk, endosperm, ovule, seed, embryo, pods, roots, cuttings, tubers, stems, stalks, fiber (lint), square, boll, fruit, berries, nuts, flowers, leaves, bark, wood, whole plant, plant cell, plant organ, epidermis, vascular tissue, protoplast, cell culture, crown, callus culture, petiole, petal, sepal, stamen, stigma, style, bud, meristem, cambium, cortex, pith, sheath, or any group of plant cells organized into a structural and functional unit. In one preferred embodiment, the exogenous nucleic acid is expressed in a specific location or tissue of a plant, for example, epidermis, vascular tissue, meristem, cambium, cortex, pith, leaf, sheath, flower, root or seed.

A “polynucleotide” is a nucleic acid polymer of ribonucleic acid (RNA), deoxyribonucleic acid (DNA), modified RNA or DNA, or RNA or DNA mimetics (such as PNAs), and derivatives thereof, and homologues thereof. Thus, polynucleotides include polymers composed of naturally occurring nucleobases, sugars and covalent inter-nucleoside (backbone) linkages as well as polymers having non-naturally-occurring portions that function similarly. Such modified or substituted nucleic acid polymers are well known in the art and for the purposes of the present invention, are referred to as “analogues.” Oligonucleotides are generally short polynucleotides from about 10 to up to about 160 or 200 nucleotides.

“Polypeptide” does not refer to a specific length of the encoded product and, therefore, encompasses peptides, oligopeptides, and proteins. “Exogenous polypeptide” means a polypeptide that is not native to the plant cell, a native polypeptide in that modifications have been made to alter the native sequence, or a native polypeptide whose expression is quantitatively altered as a result of a manipulation of the plant cell by recombinant DNA techniques.

“Promoter” is a DNA sequence that allows the binding of RNA polymerase (including but not limited to RNA polymerase I, RNA polymerase II and RNA polymerase Ill from eukaryotes), and optionally other accessory or regulatory factors, and directs the polymerase to a downstream transcriptional start site of a nucleic acid sequence encoding a polypeptide to initiate transcription. RNA polymerase effectively catalyzes the assembly of messenger RNA complementary to the appropriate DNA strand of the coding region.

A “promoter operably linked to a heterologous gene” is a promoter that is operably linked to a gene or other nucleic acid sequence that is different from the gene to that the promoter is normally operably linked in its native state. Similarly, an “exogenous nucleic acid operably linked to a heterologous regulatory sequence” is a nucleic acid that is operably linked to a regulatory control sequence to that it is not normally linked in its native state.

“Regulatory sequence” refers to any DNA sequence that influences the efficiency of transcription or translation of any gene. The term includes sequences comprising promoters, enhancers and terminators.

“Screenable marker” is a gene whose presence results in an identifiable phenotype. This phenotype can be observed under standard conditions, altered conditions such as elevated temperature, or in the presence of certain chemicals used to detect the phenotype. The use of a screenable marker allows for the use of lower, sub-killing antibiotic concentrations and the use of a visible marker gene to identify clusters of transformed cells, and then manipulation of these cells to homogeneity. Examples of screenable markers include genes that encode fluorescent proteins that are detectable by a visual microscope such as the fluorescent reporter genes DsRed, ZsGreen, ZsYellow, AmCyan, Green Fluorescent Protein (GFP). An additional preferred screenable marker gene is lac.

“Sorghum” means Sorghum bicolor (primary cultivated species), Sorghum almum, Sorghum amplum, Sorghum angustum, Sorghum rundinaceum, Sorghum brachypodum, Sorghum bulbosum, Sorghum burmahicum, Sorghum controversum, Sorghum drummondii, Sorghum carinatum, Sorghum exstans, Sorghum grande, Sorghum halepense, Sorghum interjectum, Sorghum intrans, Sorghum laxiflorum, Sorghum leiocladum, Sorghum macrospermum, Sorghum matarankense, Sorghum miliaceum, Sorghum nigrum, Sorghum nitidum, Sorghum plumosum, Sorghum propinquum, Sorghum purpureosericeum, Sorghum stipoideum, Sorghum timorense, Sorghum trichocladum, Sorghum versicolor, Sorghum virgatum, and Sorghum vulgare (including but not limited to the variety Sorghum vulgare var. sudanens also known as sudangrass). Hybrids of these species are also of interest in the present invention as are hybrids with other members of the Family Poaceae.

“Structural gene” is a sequence that codes for a polypeptide or RNA and includes 5′ and 3′ ends. The structural gene can be from the host into which the structural gene is transformed or from another species. A structural gene usually includes one or more regulatory sequences that modulate the expression of the structural gene, such as a promoter, terminator or enhancer. Structural genes often confer some useful phenotype upon an organism comprising the structural gene, for example, herbicide resistance. A structural gene can encode an RNA sequence that is not translated into a protein, for example a tRNA or rRNA gene.

“Sugar cane” refers to any species or hybrid of the genus Saccharum, including: S. acinaciforme, S. aegyptiacum, S. alopecuroides (Silver Plume Grass), S. alopecuroideum, S. alopecuroidum (Silver Plumegrass), S. alopecurus, S. angustifolium, S. antillarum, S. arenicola, S. argenteum, S. arundinaceum (Hardy Sugar Cane (USA)), S. arundinaceum var. trichophyllum, S. asper, S. asperum, S. atrorubens, S. aureum, S. balansae, S. baldwini, S. baldwinii (Narrow Plumegrass), S. barberi (Cultivated sugar cane), S. barbicostatum, S. beccarii, S. bengalense (Munj Sweetcane), S. benghalense, S. bicorne, S. biflorum, S. boga, S, brachypogon, S. bracteatum, S. brasilianum, S. brevibarbe (Short-Beard Plume Grass), S. brevibarbe var. brevibarbe (Shortbeard Plumegrass), S. brevibarbe var. contortum (Shortbeard Plumegrass), S. brevifolium, S. brunneum, S. caducam, S. canaliculatum, S. capense, S. casi, S. caudatum, S. cayennense, S. cayennense var. gemiimim, S. cayennense var. laxiusculum, S. chinense, S. ciliare, S. coarctatum (Compressed Plumegrass), S. confertum, S. conjugatun, S. contortum, S. contortum var. contortum, S. contractum, S. cotuliferum, S. cylindricum, S. cylindricum var. contractum, S. cylindricum var. longifolium, S. deciduum, S. densum, S. diandrum, S. dissitiflorum, S. distichophyllum, S. dubium, S. ecklonii, S. edule, S. elegans, S. elephantinum, S. erianthoides, S. europaeum, S. exaltatum, S. fasciculatum, S. fastigiatum, S. fatuum, S. filifolium, S. filiforme, S. floridulun, S. formosanum, S. fragile, S. fulvum, S. fuscum, S. giganteum (sugar cane Plume Grass), S. glabrum, S. glaga, S. glaucum, S. glaza, S. grandiflorum, S. griffit ii, S. hildebrandtii, S. hirsutum, S. holcoides, S. holcoides var. warmingianum, S. hookeri, S. hybrid, S. hybridum, S. indum, S. infirmum, S. insulare, S. irritans, S. jaculatorium, S. jamaicense, S. japonicum, S. juncifolium, S. kajkaiense, S. kanashiroi, S. klagha, S. koenigii, S. laguroides, S. longifolium, S. longisetosum, S. longisetosum var. hookeri, S. longisetum, S. Iota, S. luzonicum, S. macilentum, S. macrantherum, S. maximum, S. mexicanum, S. modhara, S. monandrum, S. moonja, S. munja, S. munroanum, S. muticum, S. narenga (arenga sugar cane), S. negrosense, S. obscurum, S. occidentale, S. officinale, S. officinalis, S. officinarum (Cultivated sugar cane), S. officinarum ‘Cheribon’, S. officinarum Otaheite’, S. officinarum Tele's Smoke’(Black Magic Repellent Plant), S. officinarum L. ‘Laukona’, S. officinarum L. ‘Violaceum’, S, officinarum var. brevipedicellatum, S. officinarum var. officinarum, S. officinarum var. violaceum (Burgundy-Leaved sugar cane), S. pallidum, S. paniceum, S. panicosum, S. pappiferum, S. parviflorum, S. pedicellare, S. perrieri, S. polydactylum, S. polystachyon, S. polystachyum, S. porphyrocomum, S. procerum, S. propinquum, S. punctatum, S. rara, S. rarum, S. ravennae (Hardy Pampas Plume Grass), S. repens, S. reptans, S. ridleyi, S. robustum (Wild New Guinean Cane), S. roseum, S. rubicundum, S. rufum, S. sagittatum, S. sanguineum, S. sape, S. sara, S. scindicus, S. semidecumbens, S. sibiricum, S. sikkhnense, S. sinense (Cultivated sugar cane), S. sisca, S. sorghum, S. speciosissimum, S. sphacelatum, S. spicatum, S. spontaneum (Wild Sugar Cane), S. spontaneum var. insulare, S. spontanum, S. stenophyllum, S. stewartii, S. strictum, S. teneriffae, S. ternatum, S. thunbergii, S. tinctorium, S. tridentatum, S. trinii, S. tristachyum, S. velutinum, S. versicolor, S. viguieri, S. villosum, S. violaceum, S. wardii, S. warmingianum, S. williamsii.

“Synthetic,” when used in the context of a polynucleotide or polypeptide, refers to a molecule that is made using standard synthetic techniques, e.g., using an automated DNA or peptide synthesizer. Synthetic sequence can be a native sequence, or a modified sequence.

“Trait” refers either to the altered phenotype of interest or the nucleic acid that causes the altered phenotype of interest.

“Transformed,” “transgenic,” “modified,” and “recombinant” refer to a host organism such as a plant into which an exogenous or heterologous nucleic acid molecule has been introduced, and includes whole plants, meiocytes, seeds, zygotes, embryos, endosperm, or progeny of such plants that retain the exogenous or heterologous nucleic acid molecule but that have not themselves been subjected to the transformation process.

A “variant polynucleotide” or “variant nucleic acid sequence” means a polynucleotide having at least about 60% nucleic acid sequence identity, more preferably at least about 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% nucleic acid sequence identity and yet more preferably at least about 99% nucleic acid sequence identity with a nucleic acid sequence, such as to those disclosed in the present invention. Variants do not encompass the native nucleotide sequence.

Ordinarily, variant polynucleotides are at least about 8 nucleotides in length, often at least about 9, 10, 11, 12, 13, 14, 15, 16, 17, 18, 19, 20, 21, 22, 23, 24, 25, 26, 27, 28, 29 30, 35, 40, 45, 50, 55, 60 nucleotides in length, or even about 75-200 nucleotides in length, or more.

### EXAMPLE

The following example is meant to only exemplify the invention, not to limit it in any way. One of skill in the art can envision many variations and methods to practice the invention.

### Example 1

### Constructs for Producing Farnesol

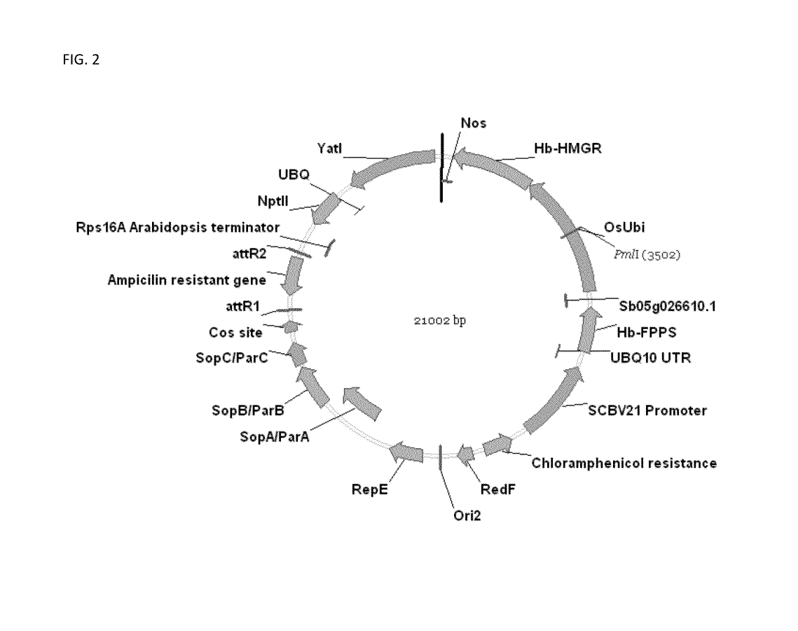

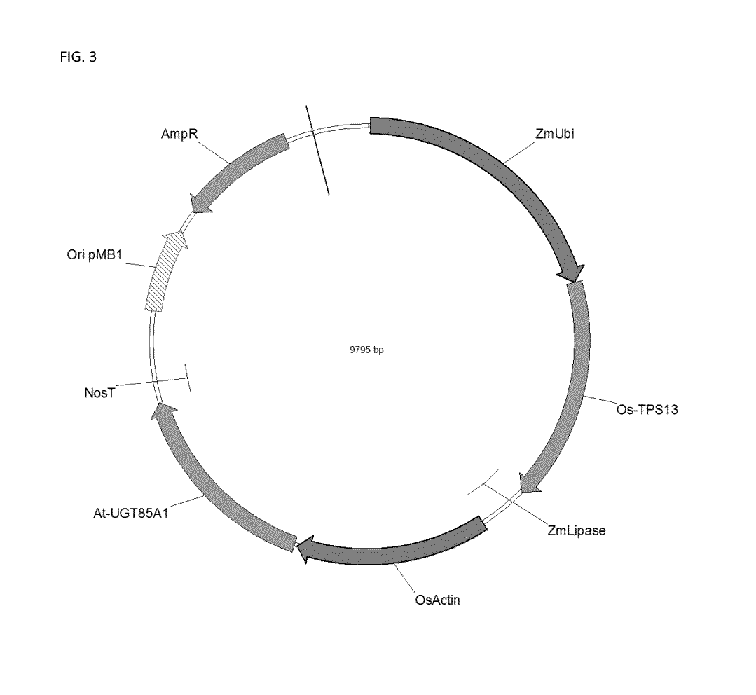

We have identified genes necessary to produce farnesol glycosides in plants. For this purpose, we will use two plasmids to transform plants according to the methods disclosed previously. The first plasmid (“Construct 1,” Table 4) will contain the rate limiting MVA pathway enzyme HMG CoA reductase (HMGR) and FPP synthase (FPPS) to increase the FPP pool. The second plasmid (“Construct 2,” Table 4) contains a farnesol synthase that will convert the FPP pool into either farnesol. This plasmid will also carry a glycosyl transferase that has broad substrate specificity to convert farnesol into farnesol glycoside. An example of Construct 1 is shown in FIG. 2, and an example of Construct 2 is shown in FIG. 3.

The constructs will be transformed into sorghum cells, and transformed cells will be selected using suitable drug selection. The selected events will be then characterized for gene expression using, for example, PCR analyses. Events showing gene expression are then analyzed for the production of farnesol glucoside.

TABLE 4
Constructs

| | Construct 1 | | Construct 2 | |
|---|---|---|---|---|
| Description | Promoter | Gene | Promoter | Gene |
| Constitutive expression | Os-UBI | HbHMGR | Zm-UBI | Os-TPS13 |
| of farnesol synthase | ScBV | HbFPPS | Os-ACT | At- |
| and glycosyltransferase | YAT1 | NPTII | | UGT85A1 |
| to sequester farnesol | | | | |
| in vacuoles | | | | |

TABLE OF SELECTED ABBREVIATIONS

| Abbreviation | Term |
|---|---|
| ACC | 1-aminocyclopropane-1- |
| | carboxylic acid |
| AMV | alfalfa mosaic virus |
| AS | acetosyringone |
| BDS | bovine serum albumin |
| BION ® | benzo (1,2,3) thiadiazole-7- |
| | carbothioic acid S-methyl |
| | ester |
| CaMV | cauliflower mosaic virus |
| DMAPP | dimethylallyl pyrophosphate |
| EDTA | ethylenediaminetetraacetic |
| | acid |
| ELISA | enzyme-linked |
| | immunosorbant assay |
| EMCV | encephalomyocarditis virus |
| FPP | farnesyl pyrophosphate |
| G418 | GENETICIN ® |
| GC-MS | gas chromatography-mass |
| | spectrometry |
| GFG | green fluorescent protein |
| GGPP | geranylgeranyl |
| | pyrophosphate |
| HMG-CoA | 3-hydroxy-3-methyl-glutaryl- |
| reductase | CoA reductase |
| IPP | isopentenyl pyrophosphate |
| MCMV | maize chlorotic mottle virus |
| MDMV | maize dwarf mosaic virus |
| MEP | methylerythritol phosphate |
| | pathway |
| MS | Murashige and Skoog |
| MVA | mevalonic acid pathway |
| NAA | α-naphthaleneacetic acid |
| NIR | near infrared |
| NMR | nuclear magnetic resonance |
| PEG | polyethylene glycol |
| PGR | plant growth regulator |
| PVP | polyvinylpyrrolidone |
| SDS | sodium dodecyl sulfate |
| SPME | solid phase microextraction |
| SRM | shoot regeneration medium |
| SSC | sodium chloride/sodium citrate |
| TEV | tobacco etch virus |
| TMV | tobacco mosaic virus |
| Tris | tris(hydroxymethyl)aminomethane |
| UDPG:glucosyl | uridine diphosphate |
| transferase | glucose:glucosyl transferase |

### BIBLIOGRAPHY

Aharoni, A., A. P. Giri, S. Deuerlein, F. Griepink, W. J. de Kogel, F. W. Verstappen, H. A. Verhoeven, M. A. Jongsma, W. Schwab, and H. J. Bouwmeester. 2003. Terpenoid metabolism in wild-type and transgenic Arabidopsis plants. Plant Cell. 15:2866-2884.
Ananda, N., and P. V. Vadlani. 2010a. Fiber Reduction and Lipid Enrichment in Carotenoid-Enriched Distillers Dried Grain with Solubles Produced by Secondary Fermentation of Phaffia rhodozyma and Sporobolomyces roseus. Journal of Agricultural and Food Chemistry. 58:12744-12748.
Ananda, N., and P. V. Vadlani. 2010b. Production and optimization of carotenoid-enriched dried distiller's grains with solubles by Phaffia rhodozyma and Sporobolomyces roseus fermentation of whole stillage. Journal of industrial microbiology & biotechnology. 37:1183-1192.
Aoyama, T., and N. H. Chua. 1997. A glucocorticoid-mediated transcriptional induction system in transgenic plants. Plant J. 11:605-612.
Arce, A., M. J. Earle, H. Rodriguez, K. R. Seddon, and A. Soto. 2008. 1-Ethyl-3-methylimidazolium bis{(trifluoromethyl)sulfonyl}amide as solvent for the separation of aromatic and aliphatic hydrocarbons by liquid extraction-extension to C-7- and C-8-fractions. Green Chemistry. 10:1294-1300.
Arce, A., A. Pobudkowska, O. Rodriguez, and A. Soto. 2007. Citrus essential oil terpenless by extraction using 1-ethyl-3-methylimidazolium ethylsulfate ionic liquid: Effect of the temperature. Chemical Engineering Journal. 133:213-218.
Ausubel, F. M. 1987. Current protocols in molecular biology. Greene Publishing Associates; J. Wiley, order fulfillment, Brooklyn, N.Y. Media, Pa. 2 v. (loose-leaf)
Ausubel, F. M., R. Brent, R. E. Kingston, D. D. Moore, J. G. Seidman, J. A. Smith, and K. Struhl. 1987. Current protocols in molecular biology. John Wiley & Sons, New York.
Blakeslee, J., K. Cornish, O. Crasta, O. Folkerts, D. Jessen, and R. B. Nair. 2013. ENGINEERING PLANTS WITH RATE-LIMITING FARNESENE METABOLIC GENES, PCT.
Board, N. B. 2011. BioDiesel.
Bohlmann, J., and C. I. Keeling. 2008. Terpenoid biomaterials. Plant J. 54:656-669.
Bonner, J. 1943. Effects of temperature on rubber accumulation by the Guayule plant. Bot Gaz. 105:233-243.
Brijwani, K., H. S. Oberoi, and P. V. Vadlani. 2010. Production of a cellulolytic enzyme system in mixed-culture solid-state fermentation of soybean hulls supplemented with wheat bran. Process Biochemistry. 45:120-128.
Buchardt, O., P. Nielsen, and R. Berg. 1992. PEPTIDE NUCLEIC ACIDS.
Burgess-Brown, N., S. Sharma, F. Sobott, C. Loenarz, U. Oppermann, and O. Gileadi. 2008. Codon optimization can improve expression of human genes in Escherichia coli: A multi-gene study. Protein Expr. Purif. 59:94-102.
Callis, J., M. Fromm, and V. Walbot. 1987. Introns increase gene expression in cultured maize cells. Genes Dev. 1:1183-1200.
Capecchi, M. R. 1980. High efficiency transformation by direct microinjection of DNA into cultured mammalian cells. Cell. 22:479-488.
Cheng, A. X., Y. G. Lou, Y. B. Mao, S. Lu, L. J. Wang, and X. Y. Chen. 2007a. Plant terpenoids: Biosynthesis and ecological functions. J Integr Plant Biol. 49:179-186.
Cheng, A. X., C. Y. Xiang, J. X. Li, C. Q. Yang, W. L. Hu, L. J. Wang, Y. G. Lou, and X. Y. Chen. 2007b. The rice (E)-beta-caryophyllene synthase (OsTPS3) accounts for the major inducible volatile sesquiterpenes. Phytochemistry. 68:1632-1641.
Clapp, D. W. 1993. Somatic gene therapy into hematopoietic cells. Current status and future implications. Clinics in perinatology. 20:155-168.
Coffelt, T. A., F. S, Nakayama, D. T. Ray, K. Cornish, C. M. McMahan, and C. F. Williams. 2009. Plant population, planting date, and germplasm effects on guayule latex, rubber, and resin yields. Ind Crop Prod. 29:255-260.
Connor, M. R., and S. Atsumi. 2010. Synthetic biology guides biofuel production. J Biomed Biotechnol. 2010.
Cornish, K., and R. A. Backhaus. 2003. Induction of rubber transferase activity in guayule (Parthenium argentatum Gray) by low temperatures. Ind Crop Prod. 17:83-92.
Curiel, D. T., S. Agarwal, E. Wagner, and M. Cotten. 1991. Adenovirus enhancement of transferrin-polylysine-mediated gene delivery. Proceedings of the National Academy of Sciences of the United States of America. 88:8850-8854.
Curiel, D. T., E. Wagner, M. Cotten, M. L. Birnstiel, S. Agarwal, C. M. Li, S. Loechel, and P. C. Hu. 1992. High-efficiency gene transfer mediated by adenovirus coupled to DNA-polylysine complexes. Human gene therapy. 3:147-154.
Dafny-Yelin, M., and T. Tzfira. 2007. Delivery of multiple transgenes to plant cells. Plant physiology. 145:1118-1128.
Demyttenaere, J. C. R., R. M. Morina, N. De Kimpe, and P. Sandra. 2004. Use of headspace solid-phase microextraction and headspace sorptive extraction for the detection of the volatile metabolites produced by toxigenic Fusarium species. Journal of Chromatography a. 1027:147-154.
Dierig, D. A., D. T. Ray, T. A. Coffelt, F. S, Nakayama, G. S. Leake, and G. Lorenz. 2001. Heritability of height, width, resin, rubber, and latex in guayule (Parthenium argentatum). Ind Crop Prod. 13:229-238.
Edris, A. E., R. Chizzola, and C. Franz. 2008. Isolation and characterization of the volatile aroma compounds from the concrete headspace and the absolute of Jasminum sambac (L.) Ait. (Oleaceae) flowers grown in Egypt. European Food Research and Technology. 226:621-626.
Eglitis, M. A., and W. F. Anderson. 1988. Retroviral vectors for introduction of genes into mammalian cells. BioTechniques. 6:608-614.
Eglitis, M. A., P. W. Kantoff, D. B. Kohn, E. Karson, R. C. Moen, C. D. Lothrop, Jr., R. M. Blaese, and W. F. Anderson. 1988. Retroviral-mediated gene transfer into hemopoietic cells. Advances in experimental medicine and biology. 241:19-27.
Fino, J. 1995. HAPTENS, TRACERS, IMMUNOGENS AND ANTIBODIES FOR
CARBAZOLE AND DIBENZOFURAN DERIVATIVES.
Fischer, C. R., D. Klein-Marcuschamer, and G. Stephanopoulos. 2008. Selection and optimization of microbial hosts for biofuels production. Metabolic Engineering. 10:295-304.
Fromm, M., L. P. Taylor, and V. Walbot. 1985. Expression of genes transferred into monocot and dicot plant cells by electroporation. Proceedings of the National Academy of Sciences of the United States of America. 82:5824-5828.
Fynan, E. F., R. G. Webster, D. H. Fuller, J. R. Haynes, J. C. Santoro, and H. L. Robinson. 1993. DNA vaccines: protective immunizations by parenteral, mucosal, and gene-gun inoculations. Proceedings of the National Academy of Sciences of the United States of America. 90:11478-11482.
Graham, F. L., and A. J. van der Eb. 1973. Transformation of rat cells by DNA of human adenovirus 5. Virology. 54:536-539.
Hammond, B., Polhamus, L G. 1965. Research on guayule (Parthenium argentatum): 1942-1959. Vol. Technical Bulletin 1327. USDA-ARS, editor. 157.
Hernanz, D., V. Gallo, A. F. Recamales, A. J. Melendez-Martinez, and F. J. Heredia. 2008. Comparison of the effectiveness of solid-phase and ultrasound-mediated liquid-liquid extractions to determine the volatile compounds of wine. Talanta. 76:929-935.
Johnston, S. A., and D. C. Tang. 1994. Gene gun transfection of animal cells and genetic immunization. Methods in cell biology. 43 Pt A:353-365.
Jorgensen, R., C. Snyder, and J. Jones. 1987. T-DNA is organized predominatly in inverted repeat structures in plants transformed with Agrobacterium tumefaciens C58 derivatives. Mol. Gen. Genetics. 207:471-477.
Lai, S. M., I. W. Chen, and M. J. Tsai. 2005. Preparative isolation of terpene trilactones from Ginkgo biloba leaves. Journal of Chromatography a. 1092:125-134.
Lanot, A., D. Hodge, E. K. Lim, F. E. Vaistij, and D. J. Bowles. 2008. Redirection of flux through the phenylpropanoid pathway by increased glycosylation of soluble intermediates. Planta. 228:609-616.
Lim, E. K. 2005. Plant glycosyltransferases: their potential as novel biocatalysts. Chemistry. 11:5486-5494.
Lin, Y., and S. Tanaka. 2006. Ethanol fermentation from biomass resources: current state and prospects. Appl Microbiol Biotechnol. 69:627-642.
Lu, L., M. Xiao, D. W. Clapp, Z. H. Li, and H. E. Broxmeyer. 1993. High efficiency retroviral mediated gene transduction into single isolated immature and replatable CD34(3+) hematopoietic stem/progenitor cells from human umbilical cord blood. The Journal of experimental medicine. 178:2089-2096.
Lucker, J., H. J. Bouwmeester, W. Schwab, J. Blaas, L. H. van der Plas, and H. A. Verhoeven. 2001. Expression of Clarkia S-linalool synthase in transgenic petunia plants results in the accumulation of S-linalyl-beta-D-glucopyranoside. Plant J. 27:315-324.
Mattingly, P. 1995. HAPTENS, TRACERS, IMMUNOGENS AND ANTIBODIES FOR 3-PHENYL-A-ADAMANTANEACETIC ACIDS.
Mookdasanit, J., H. Tamura, T. Yoshizawa, T. Tokunaga, and K. Nakanishi. 2003. Trace volatile components in essential oil of Citrus sudachi by means of modified solvent extraction method. Food Science and Technology Research. 9:54-61.
Needleman, S. B., and C. D. Wunsch. 1970. A general method applicable to the search for similarities in the amino acid sequence of two proteins. Journal of molecular biology. 48:443-453.
Newell, R. 2011. Annual Energy Outlook 2011, Reference Case.
Nigam, P. S., and A. Singh. 2011. Production of liquid biofuels from renewable resources. Progress in Energy and Combustion Science. 37:52-68.
Oberoi, H. S., P. V. Vadlani, R. L. Madl, L. Saida, and J. P. Abeykoon. 2010. Ethanol Production from Orange Peels: Two-Stage Hydrolysis and Fermentation Studies Using Optimized Parameters through Experimental Design. Journal of Agricultural and Food Chemistry. 58:3422-3429.
Omirulleh, S., M. Abraham, M. Golovkin, I. Stefanov, M. K. Karabaev, L. Mustardy, S. Morocz, and D. Dudits. 1993. Activity of a chimeric promoter with the doubled CaMV 35S enhancer element in protoplast-derived cells and transgenic plants in maize. Plant molecular biology. 21:415-428.
Pearson, C. H., K. Cornish, C. M. McMahan, D. J. Rath, and M. Whalen. 2010. Natural rubber quantification in sunflower using an automated solvent extractor. Ind Crop Prod. 31:469-475.
Pechous, S. W., C. B. Watkins, and B. D. Whitaker. 2005. Expression of alpha-farnesene synthase gene AFS1 in relation to levels of alpha-farnesene and conjugated trienols in peel tissue of scald-susceptible ‘Law Rome’ and scald-resistant ‘Idared’ apple fruit. Postharvest Biology and Technology. 35:125-132.
Peralta-Yahya, P., and J. Keasling. 2010. Advanced biofuel production in microbes. Biotechnol J. 5:147-162.
Petrasovits, L. A. P., M. P.; Nielsen, L. K.; Brumbley, S. M. 2007. Production of polyhydroxybutyrate in sugarcane. Plant Biotechnology Journal. 5:162-172.
Pourbafrani, M., G. Forgacs, I. S. Horvath, C. Niklasson, and M. J. Taherzadeh. 2010. Production of biofuels, limonene and pectin from citrus wastes. Bioresour Technol. 101:4246-4250.
Pulido, P., C. Perello, and M. Rodriguez-Concepcion. 2012. New insights into plant isoprenoid metabolism. Mol. Plant. 5:964-967.
RFA. 2011. Renewable Fuels Association—ethanol facts.
Rogers, S. G., H. J. Klee, R. B. Horsch, and R. T. Fraley. 1987. Improved vectors for plant transformation: expression cassette vectors and new selectable markers. Methods Enzymol. 153:253-277.
Rout, P. K., S. N. Naika, and Y. R. Rao. 2008. Subcritical CO2 extraction of floral fragrance from Quisqualis indica. Journal of Supercritical Fluids. 45:200-205.
Ruth, J. 1990. Ruth, J. 1990. SINGLE-STRANDED LABELED OLIGONUCLEOTIDES, REACTIVE
MONOMERS AND METHODS OF SYNTHESIS.
Salvucci, M. E., T. A. Coffelt, and K. Cornish. 2009. Improved methods for extraction and quantification of resin and rubber from guayule. Ind Crop Prod. 30:9-16.
Serrano, A., and M. Gallego. 2006. Continuous microwave-assisted extraction coupled on-line with liquid-liquid extraction: Determination of aliphatic hydrocarbons in soil and sediments. Journal of Chromatography a. 1104:323-330.
Spielmann, A., and R. Simpson. 1986. T-DNA structure in transgenic tobacco plants with multiple independent integration sites Mol. Gen. Genetics. 205:34-41.
Tipton, J. L., and E. C. Gregg. 1982. Variation in Rubber Concentration of Native Texas Guayule. Hortscience. 17:742-743.
Unger, E. A., J. M. Hand, A. R. Cashmore, and A. C. Vasconcelos. 1989. Isolation of a cDNA encoding mitochondrial citrate synthase from Arabidopsis thaliana. Plant Mol. Biol. 13:411-418.
van der Krol, A. R., J. N. Mol, and A. R. Stuitje. 1988. Modulation of eukaryotic gene expression by complementary RNA or DNA sequences. Biotechniques. 6:958-976.
Veatch, M. E., D. T. Ray, C. J. D. Mau, and K. Cornish. 2005. Growth, rubber, and resin evaluation of two-year-old transgenic guayule. Ind Crop Prod. 22:65-74.
Wagner, E., K. Zatloukal, M. Cotten, H. Kirlappos, K. Mechtler, D. T. Curiel, and M. L. Birnstiel. 1992. Coupling of adenovirus to transferrin-polylysine/DNA complexes greatly enhances receptor-mediated gene delivery and expression of transfected genes. Proceedings of the National Academy of Sciences of the United States of America. 89:6099-6103.
Wang, C., S. H. Yoon, A. A. Shah, Y. R. Chung, J. Y. Kim, E. S. Choi, J. D. Keasling, and S. W. Kim. 2010. Farnesol production from Escherichia coli by harnessing the exogenous mevalonate pathway. Biotechnol Bioeng. 107:421-429.
Whitworth, J. W., EE. 1991. Guayule natural rubber: a technical publication with emphasis on recent findings. USDA-ARS, editor. Office of Arid Land Studies, The University of Arizona, Tuscon. 445.
Wong, T. K., and E. Neumann. 1982. Electric field mediated gene transfer. Biochemical and biophysical research communications. 107:584-587.
Wu, S., M. Schalk, A. Clark, R. B. Miles, R. Coates, and J. Chappell. 2006. Redirection of cytosolic or plastidic isoprenoid precursors elevates terpene production in plants. Nat. Biotechnol. 24:1441-1447.
Zatloukal, K., E. Wagner, M. Cotten, S. Phillips, C. Plank, P. Steinlein, D. T. Curiel, and M. L. Birnstiel. 1992. Transferrinfection: a highly efficient way to express gene constructs in eukaryotic cells. Annals of the New York Academy of Sciences. 660:136-153.
Zhan, X., D. Wang, M. R. Tuinstra, S. Bean, P. A. Seib, and X. S. Sun. 2003. Ethanol and lactic acid production as affected by sorghum genotype and location. Ind Crop Prod. 18:245-255.
Zheng, C. H., T. H. Kim, K. H. Kim, Y. H. Leem, and H. J. Lee. 2004. Characterization of potent aroma compounds in Chrysanthemum coronarium L. (Garland) using aroma extract dilution analysis. Flavour and Fragrance Journal. 19:401-405.
Zini, C. A., K. D. Zanin, E. Christensen, E. B. Caramao, and J. Pawliszyn. 2003. Solid-phase microextraction of volatile compounds from the chopped leaves of three species of Eucalyptus. Journal of Agricultural and Food Chemistry. 51:2679-2686.
Zon, G. 1988. Oligonucleotide analogues as potential chemotherapeutic agents. Pharm Res. 5:539-549.

1. A method of accumulating at least one sesquiterpene in a plant cell comprising
a) expressing a transgene encoding an exogenous polypeptide that hydroxylates at least one sesquiterpene in the plant cell, and
b) accumulating the hydroxylated sesquiterpene within the plant cell, wherein the hydroxylated sesquiterpene is less volatile than at least one unhydroxylated sesquiterpene and thereby accumulates within the plant cell.

2. The method of claim 1, wherein the plant cell produces a greater amount of the at least one sesquiterpene when compared to that produced by a non-transgenic cell of the same genotype that does not express the transgene.

3. The method of claim 1, wherein the plant cell is a transgenic plant cell engineered to produce elevated amounts of the at least one sesquiterpene when compared to the amount of at least one sesquiterpene produced by a non-transgenic cell of the same genotype in the absence of expression of the exogenous polypeptide.

4. The method of claim 2, wherein the at least one sesquiterpene is farnesene.

5. The method of claim 4, wherein the hydroxylated sesquiterpene is farnesol.

6. The method of claim 5, wherein the exogenous polypeptide is a farnesol synthase or a cytochrome P450 enzyme.

7. The method of claim 6, wherein the exogenous polypeptide comprises an amino acid sequence having at least 70% sequence identity to SEQ ID NO:2 or SEQ ID NO:10, or an active fragment thereof.

8. (canceled)

9. The method of claim 6, wherein the exogenous polypeptide is encoded by the nucleic acid sequence of SEQ ID NO:1 or SEQ ID NO:9 respectively.

10. A method of accumulating farnesol in a plant cell comprising
a) expressing a transgene encoding an exogenous farnesyl diphosphate synthase polypeptide, and
b) accumulating the farnesol within the plant cell.

11. The method of claim 10, wherein the exogenous farnesyl diphosphate synthase polypeptide comprises an amino acid sequence having at least 70% sequence identical to SEQ ID NO:4, or an active fragment thereof.

12. (canceled)

13. The method of claim 10, wherein the exogenous farnesyl diphosphate synthase polypeptide is encoded by a polynucleotide of SEQ ID NO:3.

14. A method of accumulating farnesol glycoside in a plant cell comprising
a) expressing a first transgene encoding an exogenous farnesyl diphosphate synthase polypeptide to accumulate farnesol,
b) expressing a second transgene encoding an exogenous glycosyl transferase that modifies the farnesol to farnesol glycoside,
c) accumulating the farnesol glycoside within the plant cell, wherein the farnesol glycoside is less volatile than farnesol.

15. The method of claim 14, wherein the exogenous glycosyl transferase is a UDPG:glucosyl transferase polypeptide, and the farnesol glycoside is a farnesol glucoside.

16. The method of claim 15, wherein the UDPG:glucosyl transferase polypeptide comprises an amino acid sequence having at least 70% sequence identity to SEQ ID NOs:6, 12, 14, 16, 18, 20, 22, or 24, or an active fragment thereof.

17. (canceled)

18. The method of claim 15, wherein the UDPG:glucosyl transferase polypeptide is encoded by the nucleic acid sequence of SEQ ID NOs:5, 11, 13, 15, 17, 19, 21, or 23.

19. The method of claim 14, further comprising expressing in the transgenic plant cell a third transgene encoding an exogenous linalool synthase.

20. The method of claim 19, wherein the exogenous linalool synthase polypeptide comprises an amino acid sequence having at least 70% sequence identity to SEQ ID NO:8, or an active fragment thereof.

21. (canceled)

22. The method of claim 19, wherein the exogenous linalool synthase polypeptide is encoded by a nucleic acid sequence of SEQ ID NO:7.

23-89. (canceled)

90. The method of claim 1, wherein the plant cell is selected from the group consisting of a sorghum plant cell, a sugar cane plant cell, and a guayule plant cell.

91. The method of claim 10, wherein the plant cell is selected from the group consisting of a sorghum plant cell, a sugar cane plant cell, and a guayule plant cell.

92. The method of claim 14, wherein the plant cell is selected from the group consisting of a sorghum plant cell, a sugar cane plant cell, and a guayule plant cell.


SEQUENCE LISTING (24 sequences)

SEQ ID NO: 1 - DNA, 1623 bp, Oryza sativa
atggcgccggctttccaccctgcgatattcggtgacttcttcatcaacaacgtccaaccatcacccaaagagtcagatgaatggatggaagagagagtagatcagttggttgaggaggtaggtagaatgcttgaggtttgcaaggatgatgtggtaaagcaaatgaacctggtggatgtgctccaacgtttgggaatagatcatcattttgaggagcagatcgacaccattctaaaaaatattcatagagctgagttcaatagctctgacctttacgaggttgcccttcggtttcgcttacttaggaaacaagggtattgggtctcgccagatgaattcaacaaattcaaagctgaagatgggagctttagtagtgatgacataactaatgatccgaagggcttgttaagtttatacaacgcagctcaccttctaactcacaacgagaaagcacttgaagaagctatattgtttgcaaggcatcacctacaattattgagaggcaacctcgcgtacccattggatgaacaagtaacacgtgcccttgagataccgttaccaaggaccatgaagagagtagaggtgctaaattatatcttcgagtatagcgctgaagagaaaatgttcaatccctctattttggagctagccgtgcttgatttcaatattctacaaaaagttcaccaaaatgaactcaaggaaatttgtcagtggtgggagaatctttcaagtgacattagactcgactacgtccgtgaacgtgtggttgagtgctatttttgtgcatacgctgcctactatgaaaaagagcacgcacgggcccgtatgatattcgccaagaggtgtatgctattttcactgctcgatgatacatacgatgtgcgtgcaactttagaggaggctcgcaagtttaacgatgctctgcaaagatgggacaagagtgacgtttcacttctaccagaggacctgaagagattttttctgagcataataagtaactttagggagtttgaggatgaattggaaccacatgagaaatatcgtaattcttacaacattaaagcgtttcaaatactatcgagcaatttcctccaagaagcagaatggtttcatcaaaactatattccatgctttactgatcatgtgactgtttccctccagaccggaggtgcaatagagttacctgttagtttaattgttggcatgggtgatatagcaactaaggaggtgttggattgggccttggctaaccctgatgctggtagggcctttgcagaggtggcacgattcatggatgacttagctgcatcacacagtgggagggacaagatggatgtggcgagcactgtggagtgttacatgaacgagcacggggtgacgagagaggtcgccgaggcgaagatcgctggaatggctgaggacgggtggaaaagcatgaaccagatacgcttcaagcaccgcgcgttcctcccgttcgtgcagcggatcgccaacctgtgcatgtccgccaccctcctataccatggcaagaagaatggcttcagcaatagcctggagctaaaggatatgttcgagagccattttgttaacccaatcccgcttaatcatatagattacgattaa

SEQ ID NO: 2 - protein, 540 aa, Oryza sativa
MAPAFHPAIFGDFFINNVQPSPKESDEWMEERVDQLVEEVGRMLEVCKDDVVKQMNLVDVLQRLGIDHHFEEQIDTILKNIHRAEFNSSDLYEVALRFRLLRKQGYWVSPDEFNKFKAEDGSFSSDDITNDPKGLLSLYNAAHLLTHNEKALEEAILFARHHLQLLRGNLAYPLDEQVTRALEIPLPRTMKRVEVLNYIFEYSAEEKMFNPSILELAVLDFNILQKVHQNELKEICQWWENLSSDIRLDYVRERVVECYFCAYAAYYEKEHARARMIFAKRCMLFSLLDDTYDVRATLEEARKFNDALQRWDKSDVSLLPEDLKRFFLSIISNFREFEDELEPHEKYRNSYNIKAFQILSSNFLQEAEWFHQNYIPCFTDHVTVSLQTGGAIELPVSLIVGMGDIATKEVLDWALANPDAGRAFAEVARFMDDLAASHSGRDKMDVASTVECYMNEHGVTREVAEAKIAGMAEDGWKSMNQIRFKHRAFLPFVQRIANLCMSATLLYHGKKNGFSNSLELKDMFESHFVNPIPLNHIDYD

SEQ ID NO: 3 - DNA, 900 bp, Escherichia coli
atggactttccgcagcaactcgaagcctgcgttaagcaggccaaccaggcgctgagccgttttatcgccccactgccctttcagaacactcccgtggtcgaaaccatgcagtatggcgcattattaggtggtaagcgcctgcgacctttcctggtttatgccactggtcatatgttcggcgttagcacaaacacactggacgcacccgctgccgccgtagagtgtatccacgcttactcattaattcatgatgatttaccggcgatggatgatgacgatctgcgtcgcggtttgccgacctgccatgtgaagtttggcgaagcaaacgcgattctcgctggtgacgctttacaaacgctggcgttctcgattctaagcgatgccgatatgccggaagtgtcagatcgcgacagaatttcgatgatttctgagctggcgagcgccagcggtattgccggaatgtgcggtggtcaggcattagatttagacgctgaaggcaaacacgtacctctggacgcgcttgagcgtattcatcgtcataaaaccggcgcattgattcgcgccgccgttcgccttggtgcattaagcgccggagataaaggacgtcgtgctctgccagtactcgacaagtacgcagagagcatcggccttgccttccaggttcaggatgacatcctggatgtggtgggagatactgcaacgttgggaaaacgccagggtgccgaccagcaacttggtaaaagtacctaccctgcacttctgggtcttgagcaagcccggaagaaagcccgggatctgatcgacgatgcccgtcagtcgctgaaacaactggctgaacagtcactcgatacctcggcactggaagcgctagcggactacatcatccagcgtaataaataa

SEQ ID NO: 4 - protein, 299 aa, Escherichia coli
MDFPQQLEACVKQANQALSRFIAPLPFQNTPVVETMQYGALLGGKRLRPFLVYATGHMFGVSTNTLDAPAAAVECIHAYSLIHDDLPAMDDDDLRRGLPTCHVKFGEANAILAGDALQTLAFSILSDADMPEVSDRDRISMISELASASGIAGMCGGQALDLDAEGKHVPLDALERIHRHKTGALIRAAVRLGALSAGDKGRRALPVLDKYAESIGLAFQVQDDILDVVGDTATLGKRQGADQQLGKSTYPALLGLEQARKKARDLIDDARQSLKQLAEQSLDTSALEALADYIIQRNK

SEQ ID NO: 5 - DNA, 1724 bp, Arabidopsis thaliana
aaaccgacacaataacaacaacaacaacaacaaaaaaaaaaaaaaaaaaggccaacgttcagtgatctaggctatgaagattacaaaaccacatgtggccatgttcgctagccccggaatgggccacatcatcccggtgatcgagctcggaaaacgcttagctggttcccacggcttcgatgtcaccattttcgtccttgaaaccgacgcagcctcagctcaatctcaattccttaactcaccaggctgcgacgcggcccttgttgatatcgttggcctcccaacgcccgatatctccggtttagtcgacccatcagccttttttgggatcaagctcttggtcatgatgcgtgagaccattcctaccatccggtcaaagatagaggagatgcaacacaaaccaacggctctgatcgtagacttgtttggtttggacgcgataccgctcggtggtgagttcaacatgttgacttatatcttcatcgcttcaaacgcacgttttctcgcggtggctttgtttttcccaacgttggacaaagacatggaagaagagcacataatcaagaagcaacctatggttatgcctggatgtgaaccggttcggtttgaagatacacttgaaacattccttgacccaaacagccaactctaccgggaatttgttcctttcggttcggttttcccaacgtgtgatggtattattgtgaatacatgggatgatatggagcccaaaactttgaaatctcttcaagacccaaagctcttgggtcgaattgctggtgtaccggtttatccaattggtcctttgtctagaccggttgatccatctaaaactaatcatccggttttggattggttaaacaaacagccggacgagtcggtactttacatttcatttggaagcggtggctctctctcggctaaacaactaaccgaattggcttggggacttgagatgagtcagcaacggttcgtttgggtggttcgacccccggtggacggttcagcttgcagtgcatatttatccgctaacagtggtaaaatacgagacggtacacctgattatctcccggaaggttttgttagccggactcatgagagaggctttatggtctcttcttgggctccccaagcggagatcttggcccaccaagccgtaggtgggtttctaactcactgcggttggaattcgattctcgagagcgtcgttggtggcgttccgatgatcgcgtggccactttttgcggagcagatgatgaacgcgacactcctcaacgaagagcttggcgttgccgtccgctctaagaaactaccgtcggagggagtgattacgagggcggagatcgaggcgttggtgagaaagatcatggtggaggaggaaggtgctgagatgagaaagaagataaagaagctgaaagagaccgctgccgaatcgctgagttgcgacggtggagtggcgcatgaatcgttgtcaagaatcgccgacgagagcgagcatcttttggagcgtgtcaggtgcatggcacgtggtgcctaggaacgcttaccgtttatagtatgttacttataaactgcgtgtcgttttacccttttgggtatcttgtattatatagtttccgagtcttggttgccacatggtatcacgtggttttgttttggtcaatgctagtccctatcagtaatgtattttacaatgtaaaataaataattagtgtttaatttgt

SEQ ID NO: 6 - protein, 487 aa, Arabidopsis thaliana
MKITKPHVAMFASPGMGHIIPVIELGKRLAGSHGFDVTIFVLETDAASAQSQFLNSPGCDAALVDIVGLPTPDISGLVDPSAFFGIKLLVMMRETIPTIRSKIEEMQHKPTALIVDLFGLDAIPLGGEFNMLTYIFIASNARFLAVALFFPTLDKDMEEEHIIKKQPMVMPGCEPVRFEDTLETFLDPNSQLYREFVPFGSVFPTCDGIIVNTWDDMEPKTLKSLQDPKLLGRIAGVPVYPIGPLSRPVDPSKTNHPVLDWLNKQPDESVLYISFGSGGSLSAKQLTELAWGLEMSQQRFVWVVRPPVDGSACSAYLSANSGKIRDGTPDYLPEGFVSRTHERGFMVSSWAPQAEILAHQAVGGFLTHCGWNSILESVVGGVPMIAWPLFAEQMMNATLLNEELGVAVRSKKLPSEGVITRAEIEALVRKIMVEEEGAEMRKKIKKLKETAAESLSCDGGVAHESLSRIADESEHLLERVRCMARGA

SEQ ID NO: 7 - DNA, 1710 bp, Arabidopsis thaliana
atggccttaatagctaccaaaataagttcacgttcttgttttgtttctgcttatccaaacaattctccaacgtttctgatatccaaatttcccaatacggtcgattctttgtcgcctgcaaatactgctaaaagatcgatcttgagaaacgtccatgcttcagtttctaacccttcgaagcagtttcataataagacatccttagaatattcacacgagttgaatatcaagaagatcaagaatatactaagtgcaaacgtagatgttccatccgagaacctggagatgatcgatgtcattcaaagtttaggcactgatctccattttcgacaagggatcgagcaaacccttcacatgatttacaaagaaggcctccaattcaacggtgatctccatgagatagcgcttcgctttcgattgctgagacaagagggtcactatgttcaagaaagtattttcaaaaacatcctagacaagaagggtggatttaaagacgtagtaaaaaatgacgtcaagggtctaacagaattgtttgaagcttctgagctccgtgtagaaggtgaagaaacactcgacggtgccagagaattcacatatagccgccttaatgagctttgctcaggtagagaaagtcatcaaaagcaagagataatgaagtctttggcgcaacctcgccacaaaaccgtaagaggattaacgtccaagaggttcacaagcatgatcaaaatcgcgggtcaagaagatccagaatggttacagtctctattacgagtggcggagatcgattccattaggctaaagtcattgactcaaggagaaatgtctcaaacatttaaatggtggacagaacttggtttagaaaaagatgtggagaaggcaagaagccagccgttaaaatggcatacgtggtccatgaaaattcttcaagatccgaccttaaccgaacaaaggcttgatcttaccaaaccaatatcgcttgtttatgttatagatgacattttcgatgtctatggggagctagaagaactaaccatcttcacacgagttgttgagagatgggatcataaggggcttaagacgctacccaaatacatgagggtttgttttgaagctctagatatgatcacaacggagattagcatgaagatctacaaatcacatggttggaacccgacatacgctcttcgacaatcgtgggcaagtttgtgtaaagcattcttggtagaagcaaagtggtttaattcgggttacttacccaccactgaagagtatatgaagaatggggttgtgagttcaggtgttcatttagtgatgcttcatgcctatatcttgttaggcgaagaactaacaaaagagaaagtcgaactaatagagagtaacccggggattgtatcatctgcagctacaattctcaggctctgggatgatctcggaagtgccaaggatgagaaccaagatggaactgatggatcatatgtagagtgttacctgaacgagtacaagggatcaactgttgatgaagcaagaacacatgttgcccagaagatatctagagcatggaaacgcttgaacagggagtgtctgaatccatgtccattctcaagatcattctcaaaagcttgtctcaacattgcaagaacagttcctttaatgtacagctatgatgatgatcaacgacttcccgacgaatatctcaagtctctaatgtaa

SEQ ID NO: 8 - protein, 569 aa, Arabidopsis thaliana
MALIATKISSRSCFVSAYPNNSPTFLISKFPNTVDSLSPANTAKRSILRNVHASVSNPSKQFHNKTSLEYSHELNIKKIKNILSANVDVPSENLEMIDVIQSLGTDLHFRQGIEQTLHMIYKEGLQFNGDLHEIALRFRLLRQEGHYVQESIFKNILDKKGGFKDVVKNDVKGLTELFEASELRVEGEETLDGAREFTYSRLNELCSGRESHQKQEIMKSLAQPRHKTVRGLTSKRFTSMIKIAGQEDPEWLQSLLRVAEIDSIRLKSLTQGEMSQTFKWWTELGLEKDVEKARSQPLKWHTWSMKILQDPTLTEQRLDLTKPISLVYVIDDIFDVYGELEELTIFTRVVERWDHKGLKTLPKYMRVCFEALDMITTEISMKIYKSHGWNPTYALRQSWASLCKAFLVEAKWFNSGYLPTTEEYMKNGVVSSGVHLVMLHAYILLGEELTKEKVELIESNPGIVSSAATILRLWDDLGSAKDENQDGTDGSYVECYLNEYKGSTVDEARTHVAQKISRAWKRLNRECLNPCPFSRSFSKACLNIARTVPLMYSYDDDQRLPDEYLKSLM

SEQ ID NO: 9 - DNA, 1773 bp, Zea mays
atggccatgccagtgaagctgactcctgcctccctctcgctgaaggcggtctgctgccgcttcagctccggagggcatgcgctgcgcttcggctcgtcgctaccgtgctggaggaggacgccgacgcaacggagcacgtcgtcgtctacgacgcgccctgcggctgaggttagctctggcaaaagcaagcagcacgatcaagaagcatcggaggctacgataagacagcagctccagctagtcgatgtgcttgagaacatggggatttctcggcattttgctgctgaaatcaaatgcatccttgacaggacatacagaagttggttacagagacatgaggaaattatgctggacacaatgacctgtgcgatggcatttcgtattctaaggttgaatggatacaatgtctcttctgatgagttgtatcatgttgttgaagcttccggactccataattcacttggaggatatctcaatgatacaagaaccttgttagaattacacaaggcctcgacagttagtatctctgaagatgagtctatcctggatagcataggctcaaggtcacgtaccttactgagggaacaactagagtctggtggtgctctacgaaaaccttcactctttaaagaggtggaacatgctctggacggtcccttctacaccacattggaccgtctacaccataggtggaacatcgaaaatttcaatattatagagcagcacatgctagagacaccatacttgtcaaatcaacataccagtagagatattctagcgttgagtattagagacttcagttcctctcagtttacttaccagcaagaacttcaacatcttgaaagctgggtgaaagagtgcaggttagaccagctacaatttgcgcgacagaagttggcatacttctacttgtctgctgctggcaccatgttctctcctgagctgtctgatgctcgaactttgtgggccaaaaatggtgtgctcacaactattgttgacgacttctttgatgttgcgggatcaaaagaagaacttgaaaaccttgtcatgttggttgagatgtgggacgagcatcacaaagttgagttctactcagaacaagtagagattatattttcttcaatttatgactcagttaaccaacttggtgaaaaggcttctttggtacaagaccgcagtattaccaaacacctagtagaaatatggttggatttgctaaagtctatgatgacagaggtagagtggcgtttgagcaaatatgtgccaacagagaaggaatacatgataaatgcatctttaatatttggactaggccccattgtattgccagcattatattttgttgggccaaagatctcagagtctattgttaaagatccagaatatgatgaattgttcaaactgatgagcacatgtggtcgcctcttgaatgatgttcagacttttgagagggagtacaacgagggcaagttgaatagtgtttctctcctcgttcttcatggtggccccatgtccatatcagacgccaaaaggaaattacagaagcccatagacacatgcagaagagacctcctaagtttagttcttcgtgaagaaagtgttgttcctaggccctgcaaggaattattttggaaaatgtgcaaggtgtgctacttcttctactcgacgacggatgggtttagctcacaagtggagagggctaaagaagtggatgcggtgatcaatgagccactaaagctacaaggaagtcatacgctggtgtctgatgtgtga

SEQ ID NO: 10 - protein, 590 aa, Zea mays
MAMPVKLTPASLSLKAVCCRFSSGGHALRFGSSLPCWRRTPTQRSTSSSTTRPAAEVSSGKSKQHDQEASEATIRQQLQLVDVLENMGISRHFAAEIKCILDRTYRSWLQRHEEIMLDTMTCAMAFRILRLNGYNVSSDELYHVVEASGLHNSLGGYLNDTRTLLELHKASTVSISEDESILDSIGSRSRTLLREQLESGGALRKPSLFKEVEHALDGPFYTTLDRLHHRWNIENFNIIEQHMLETPYLSNQHTSRDILALSIRDFSSSQFTYQQELQHLESWVKECRLDQLQFARQKLAYFYLSAAGTMFSPELSDARTLWAKNGVLTTIVDDFFDVAGSKEELENLVMLVEMWDEHHKVEFYSEQVEIIFSSIYDSVNQLGEKASLVQDRSITKHLVEIWLDLLKSMMTEVEWRLSKYVPTEKEYMINASLIFGLGPIVLPALYFVGPKISESIVKDPEYDELFKLMSTCGRLLNDVQTFEREYNEGKLNSVSLLVLHGGPMSISDAKRKLQKPIDTCRRDLLSLVLREESVVPRPCKELFWKMCKVCYFFYSTTDGFSSQVERAKEVDAVINEPLKLQGSHTLVSDV

SEQ ID NO: 11 - DNA, 1534 bp, Arabidopsis thaliana
aaccttaggaagagccatgggtgaagaagctatagttctgtatcctgcaccaccaataggtcacttagtgtccatggttgagttaggtaaaaccatcctctccaaaaacccatctctctccatccacattatcttagttccaccgccttatcagccggaatcaaccgccacttacatctcctccgtctcctcctccttcccttcaataaccttccaccatcttcccgccgtcacaccgtactcctcctcctccacctctcgccaccaccacgaatctctcctcctagagatcctctgttttagcaacccaagtgtccaccgaactcttttctcactctctcggaatttcaatgtccgagcaatgatcatcgatttcttctgcaccgccgttttagacatcaccgctgacttcacgttcccggtttacttcttctacacctctggagccgcatgtctcgccttttccttctatctcccgaccatcgacgaaacaacccccggaaaaaacctcaaagacattcctacagttcatatccccggcgttcctccgatgaagggctccgatatgcctaaggcggtgctcgaacgagacgatgaggtctacgatgtttttataatgttcggtaaacagctctcgaagtcgtcagggattattatcaatacgtttgatgctttagaaaacagagccatcaaggccataacagaggagctctgttttcgcaatatttatccaattggaccgctcattgtaaacggaagaatcgaagatagaaacgacaacaaggcagtttcttgtctcaattggctggattcgcagccggaaaagagtgttgtgtttctctgttttggaagcttaggtttgttctcaaaagaacaggtgatagagattgctgttggtttagagaaaagtgggcagagattcttgtgggtggtccgtaatccacccgagttagaaaagacagaactggatttgaaatcactcttaccagaaggattcttaagccgaaccgaagacaaagggatggtcgtgaaatcatgggctccgcaagttccggttctgaatcataaagcagtcgggggattcgtcactcattgcggttggaattcaattcttgaagctgtttgtgctggtgtgccgatggtggcttggccgttgtacgctgagcagaggtttaatagagtgatgattgtggatgagatcaagattgcgatttcgatgaatgaatcagagacgggtttcgtgagctctacagaggtggagaaacgagtccaagagataattggggagtgtccggttagggagcgaaccatggctatgaagaacgcagccgaattagccttgacagaaactggttcgtctcataccgcattaactactttactccagtcgtggagcccaaagtgatctttgaaattaagtcttgcatcaattaataactttgtagtctctcagaatcatgtctttgggggttattcgttttactagttcaaagttatgtctctgagaccgcagatggaaagaacaatttatacc

SEQ ID NO: 12 - protein, 462 aa, Arabidopsis thaliana
MGEEAIVLYPAPPIGHLVSMVELGKTILSKNPSLSIHIILVPPPYQPESTATYISSVSSSFPSITFHHLPAVTPYSSSSTSRHHHESLLLEILCFSNPSVHRTLFSLSRNFNVRAMIIDFFCTAVLDITADFTFPVYFFYTSGAACLAFSFYLPTIDETTPGKNLKDIPTVHIPGVPPMKGSDMPKAVLERDDEVYDVFIMFGKQLSKSSGIIINTFDALENRAIKAITEELCFRNIYPIGPLIVNGRIEDRNDNKAVSCLNWLDSQPEKSVVFLCFGSLGLFSKEQVIEIAVGLEKSGQRFLWVVRNPPELEKTELDLKSLLPEGFLSRTEDKGMVVKSWAPQVPVLNHKAVGGFVTHCGWNSILEAVCAGVPMVAWPLYAEQRFNRVMIVDEIKIAISMNESETGFVSSTEVEKRVQEIIGECPVRERTMAMKNAAELALTETGSSHTALTTLLQSWSPK

SEQ ID NO: 13 - DNA, 1534 bp, Arabidopsis thaliana
atggaacaacatggcggttctagctcacagaaacctcacgcaatgtgcataccttatccagcacaaggccacatcaacccaatgctgaaactagccaagctcctccacgctagaggcttccacgtcactttcgtcaacaccgactacaaccaccgccgtatcctccaatcacgtggccctcacgctctcaacggtctcccctcgtttcgcttcgagactatccccgacggtcttccttggacagacgtcgacgctaagcaagacatgctcaagcttattgactccacaataaacaactgtttagctccattcaaagacctcatcctccggttaaactccggttctgatataccaccggttagctgtatcatctccgacgcttcaatgagcttcacaattgacgcagcggaggagcttaaaattccggtagttctcctctggaccaacagtgctactgctttaatcttgtatctccattaccaaaaactcatcgagaaagagataattcccctcaaagattcgagtgacttgaagaagcatttagagacggagattgattggataccgtcgatgaagaagattaagcttaaggattttccagatttcgtcaccacgacgaatcctcaagatccgatgattagtttcatccttcatgtaaccggaagaatcaaaagagcttctgcgatcttcatcaacactttcgaaaaactcgagcataacgttctcttatctctgcgatctcttctccctcagatctactccgttggaccgttccagattctggagaatcgcgaaatcgataagaacagcgaaatcagaaagctaggattgaatctctgggaagaagagacggagtctttggattggctagatactaaagctgagaaagctgtgatttacgtcaacttcgggagtctaacggttttgactagtgagcagatcttagagttcgcttggggtttagcgaggagcgggaaagagtttctctgggtggtgagatctggtatggtcgacggagatgattcgattcttccggcggagtttttatcggagacgaagaatcgaggaatgttaattaaaggatggtgttctcaggagaaggtactttcgcatccggcgattggaggatttttgactcactgtggatggaattcgacgttggagagtttgtacgccggtgttccgatgatctgttggccattttttgctgatcagttgacgaatcgaaagttctgttgcgaggattgggggattgggatggagatcggcgaggaggtgaagagggagagagtggagacggtggttaaagagctcatggacggagagaagggaaagaggttaagagagaaggtggtggagtggcggcgcttggcggaagaagcttcggcgccaccgttgggatcatcgtacgtgaattttgaaacggtggttaataaagtccttacatgtcacacgattagatcgacctaattgggctttccgtatccaaataggcccattagaaaaggcctgtttgtttccactatcatttggg

SEQ ID NO: 14 - protein, 489 aa, Arabidopsis thaliana
MEQHGGSSSQKPHAMCIPYPAQGHINPMLKLAKLLHARGFHVTFVNTDYNHRRILQSRGPHALNGLPSFRFETIPDGLPWTDVDAKQDMLKLIDSTINNCLAPFKDLILRLNSGSDIPPVSCIISDASMSFTIDAAEELKIPVVLLWTNSATALILYLHYQKLIEKEIIPLKDSSDLKKHLETEIDWIPSMKKIKLKDFPDFVTTTNPQDPMISFILHVTGRIKRASAIFINTFEKLEHNVLLSLRSLLPQIYSVGPFQILENREIDKNSEIRKLGLNLWEEETESLDWLDTKAEKAVIYVNFGSLTVLTSEQILEFAWGLARSGKEFLWVVRSGMVDGDDSILPAEFLSETKNRGMLIKGWCSQEKVLSHPAIGGFLTHCGWNSTLESLYAGVPMICWPFFADQLTNRKFCCEDWGIGMEIGEEVKRERVETVVKELMDGEKGKRLREKVVEWRRLAEEASAPPLGSSYVNFETVVNKVLTCHTIRST

SEQ ID NO: 15 - DNA, 1478 bp, Arabidopsis thaliana
atgggatctcatgtcgcacaaaaacaacacgtagtttgcgttccttatccggctcaaggccacatcaacccaatgatgaaagtggctaaactcctttacgccaaaggcttccatattaccttcgtcaacaccgtctacaaccacaaccgtctcctccggtcccgtgggcctaacgccgttgacgggcttccttctttccggtttgagtccatccctgacggtctacccgagactgacgtagacgtcactcaggacatccctactctttgcgagtccacaatgaagcactgtctcgctccattcaaggagcttctccggcagatcaacgcaagggatgatgttcctcctgtgagctgtatcgtatccgacggttgtatgagcttcacacttgatgctgcggaggagctcggtgtcccggaggttcttttttggacaactagtgcttgtggcttcttggcttacctttactactatcgcttcatcgagaagggattatcaccaataaaagatgagagttacttaaccaaggaacacttggacacaaaaatagactggataccatcgatgaaggacctaagactaaaagacatccctagcttcatccgaacgactaatcctgacgacatcatgctcaactttatcatccgtgaggctaaccgagccaaacgcgcttcagctatcattctcaacacgtttgatgatctcgaacacgacgttatccaatctatgaaatccattgtacctccggtttattctattggaccgttacatttactagagaaacaagagagcggcgagtatagtgaaatcggacggacaggatcgaatctttggagagaggagactgagtgtctggactggctaaacacgaaagctagaaacagtgttgtgtacgttaacttcgggagtataactgttttgagcgcaaaacagcttgtggagtttgcatggggtttggctgcaacggggaaagagtttttgtgggtgatccggccggatttagtagccggggatgaggcaatggttccaccggagtttttaacggctacggcggaccggaggatgttggcaagttggtgtcctcaagagaaagtcctttctcatccggccattggagggttcttgacgcattgcgggtggaactcgacgttggaaagtctatgcggtggagttccaatggtgtgttggccgttttttgcagagcaacaaactaattgtaagttttctcgtgacgaatgggaggttgggattgagattggtggagatgtgaagagagaagaggttgaggcggtggttagggagttgatggatgaagagaagggaaagaatatgagagagaaggcggaagagtggcggcgcttggcgaatgaagcgacggagcataagcatggttcttctaaattgaactttgagatgctcgttaataaggttcttttaggggagtagagactagtaaatatggatttcaagaagaaagg

SEQ ID NO: 16 - protein, 481 aa, Arabidopsis thaliana
MGSHVAQKQHVVCVPYPAQGHINPMMKVAKLLYAKGFHITFVNTVYNHNRLLRSRGPNAVDGLPSFRFESIPDGLPETDVDVTQDIPTLCESTMKHCLAPFKELLRQINARDDVPPVSCIVSDGCMSFTLDAAEELGVPEVLFWTTSACGFLAYLYYYRFIEKGLSPIKDESYLTKEHLDTKIDWIPSMKDLRLKDIPSFIRTTNPDDIMLNFIIREANRAKRASAIILNTFDDLEHDVIQSMKSIVPPVYSIGPLHLLEKQESGEYSEIGRTGSNLWREETECLDWLNTKARNSVVYVNFGSITVLSAKQLVEFAWGLAATGKEFLWVIRPDLVAGDEAMVPPEFLTATADRRMLASWCPQEKVLSHPAIGGFLTHCGWNSTLESLCGGVPMVCWPFFAEQQTNCKFSRDEWEVGIEIGGDVKREEVEAVVRELMDEEKGKNMREKAEEWRRLANEATEHKHGSSKLNFEMLVNKVLLGE

SEQ ID NO: 17 - DNA, 1605 bp, Arabidopsis thaliana
aaaactcacatttgttcattacaaaaagaggaaactgacaactgatttcgcgcaagagaaagaaaaatgggatctcagatcattcataactcacaaaaaccacatgtagtttgtgttccatatccggctcaaggccacatcaaccctatgatgagagtggctaaactcctccacgccagaggcttctacgtcaccttcgtcaacaccgtctacaaccacaatcgtttccttcgttctcgtgggtccaatgccctagatggacttccttcgttccgatttgagtccattgctgacggtctaccagagacagacatggatgccacgcaggacatcacagctctttgcgagtccaccatgaagaactgtctcgctccgttcagagagcttctccagcggatcaacgctggagataatgttcctccggtaagctgtattgtatctgacggttgtatgagctttactcttgatgttgcggaggagcttggagtcccggaggttcttttttggacaaccagtggctgtgcgttcctggcttatctacacttttatctcttcatcgagaagggcttatgtccgctaaaagatgagagttacttgacgaaggagtacttagaagacacggttatagattttataccaaccatgaagaatgtgaaactaaaggatattcctagcttcatacgtaccactaatcctgatgatgttatgattagtttcgccctccgcgagaccgagcgagccaaacgtgcttctgctatcattctaaacacatttgatgaccttgagcatgatgttgttcatgctatgcaatctatcttacctccggtttattcagttggaccgcttcatctcttagcaaaccgggagattgaagaaggtagtgagattggaatgatgagttcgaatttatggaaagaggagatggagtgtttggattggcttgatactaagactcaaaatagtgtcatttatatcaactttgggagcataacggttttgagtgtgaagcagcttgtggagtttgcttggggtttggcgggaagtgggaaagagtttttatgggtgatccggccagatttagtagcgggagaggaggctatggttccgccggactttttaatggagactaaagaccgcagtatgctagcgagttggtgtcctcaagagaaagtactttctcatcctgctattggagggtttttgacgcattgcgggtggaactcgatattggaaagtctttcgtgtggagttccgatggtgtgttggccattttttgctgaccagcaaatgaattgtaagttttgttgtgacgagtgggatgttgggattgagataggtggagatgtgaagagagaggaagttgaggcggtggttagagagctcatggatggagagaagggaaagaaaatgagagaaaaggcggtagagtggcagcgcttagccgagaaagcgacggaacataaacttggttcttccgttatgaattttgagacggttgttagcaagtttcttttgggacaaaaatcacaggattaactaaaatataagatgatcatcgaaatgtattattttttctataatgattactaagtaagtgtttatttt

SEQ ID NO: 18 - protein, 489 aa, Arabidopsis thaliana
MGSQIIHNSQKPHVVCVPYPAQGHINPMMRVAKLLHARGFYVTFVNTVYNHNRFLRSRGSNALDGLPSFRFESIADGLPETDMDATQDITALCESTMKNCLAPFRELLQRINAGDNVPPVSCIVSDGCMSFTLDVAEELGVPEVLFWTTSGCAFLAYLHFYLFIEKGLCPLKDESYLTKEYLEDTVIDFIPTMKNVKLKDIPSFIRTTNPDDVMISFALRETERAKRASAIILNTFDDLEHDVVHAMQSILPPVYSVGPLHLLANREIEEGSEIGMMSSNLWKEEMECLDWLDTKTQNSVIYINFGSITVLSVKQLVEFAWGLAGSGKEFLWVIRPDLVAGEEAMVPPDFLMETKDRSMLASWCPQEKVLSHPAIGGFLTHCGWNSILESLSCGVPMVCWPFFADQQMNCKFCCDEWDVGIEIGGDVKREEVEAVVRELMDGEKGKKMREKAVEWQRLAEKATEHKLGSSVMNFETVVSKFLLGQKSQD

SEQ ID NO: 19 - DNA, 1464 bp, Arabidopsis thaliana
atggaatctcatgttgttcataacgcacaaaagccacacgtagtttgcgtgccttacccggctcaaggccacatcaatccgatgctgaaagtggctaaactcctctacgctaaaggctttcacgtcaccttcgttaacactctctacaaccacaaccgtctcctccggtcccgtggtcccaacgcgctcgacgggtttccttcattccggttcgagtccatccctgacggtctaccggagactgatggcgataggacgcagcatactcctaccgtttgcatgtccattgagaaaaactgtctcgctccattcaaagagattctgcgccggatcaacgataaagatgatgttcctccagtgagttgtattgtatcggacggtgtgatgagttttactcttgacgcagccgaggaactaggtgtcccagaggttattttttggaccaatagtgcttgtggtttcatgactattctacacttttatcttttcatcgagaagggtctatctccttttaaagacgaaagttacatgtcaaaggagcatctagacacagttatagattggataccatcaatgaagaatcttaggttaaaggacatccctagctatatacgtaccacaaatcctgacaacataatgcttaatttcctcattcgagaagttgagcgatctaaacgcgctagtgctatcattctcaacacgtttgatgaactcgagcatgatgttatccagtctatgcaatctattttacctccggtttattctattgggccactccatctccttgtgaaggaagaaataaacgaggctagtgaaataggacagatgggattaaatttgtggagagaggagatggaatgtttggattggctcgatacaaaaactccaaacagtgttctttttgttaactttggatgcataacggtgatgagtgcaaaacagcttgaagaatttgcttggggtttggcggcaagtaggaaagagtttttatgggtgatccgtcctaatttagtggtgggagaggcgatggtggttcttccacaagagtttttagcggagacgatagaccggagaatgttagctagttggtgtcctcaggagaaagttctttctcatcccgcgataggagggttcttgacgcattgcgggtggaactcaacattggagagtctcgctggtggtgttccgatgatatgttggccatgtttttcggagcaaccgacgaattgtaagttttgttgtgacgagtggggagtgggtatagagattggtaaagatgtgaagagagaggaggtcgagacggtggttagagaacttatggatggagaaaaggggaaaaagctgagagaaaaggcggaagagtggcggcggttggccgaggaagcgacgaggtataaacatggttcgtcggtcatgaatcttgagacgcttatacataaagttttcttagaaaatcttagatga

SEQ ID NO: 20 - protein, 487 aa, Arabidopsis thaliana
MESHVVHNAQKPHVVCVPYPAQGHINPMLKVAKLLYAKGFHVTFVNTLYNHNRLLRSRGPNALDGFPSFRFESIPDGLPETDGDRTQHTPTVCMSIEKNCLAPFKEILRRINDKDDVPPVSCIVSDGVMSFTLDAAEELGVPEVIFWTNSACGFMTILHFYLFIEKGLSPFKDESYMSKEHLDTVIDWIPSMKNLRLKDIPSYIRTTNPDNIMLNFLIREVERSKRASAIILNTFDELEHDVIQSMQSILPPVYSIGPLHLLVKEEINEASEIGQMGLNLWREEMECLDWLDTKTPNSVLFVNFGCITVMSAKQLEEFAWGLAASRKEFLWVIRPNLVVGEAMVVLPQEFLAETIDRRMLASWCPQEKVLSHPAIGGFLTHCGWNSTLESLAGGVPMICWPCFSEQPTNCKFCCDEWGVGIEIGKDVKREEVETVVRELMDGEKGKKLREKAEEWRRLAEEATRYKHGSSVMNLETLIHKVFLENLR

SEQ ID NO: 21 - DNA, 1612 bp, Arabidopsis thaliana
gaaacaaaacttgagaggttcttactaaagttgcatcgtcatggctttcgaaaaaaacaacgaaccttttcctcttcactttgttctcttccctttcatggctcaaggccacatgattcccatggttgatattgcaaggctcttggctcagcgaggtgtgcttataacaattgtcacgacgcctcacaatgcagcaaggttcaagaatgtcctaaaccgtaccattgagtctggtttgcccatcaacctagtgcaagtcaagtttccatatcaagaagctggtctgcaagaaggacaagaaaatatggatttgcttaccacgatggagcagataacatctttctttaaagcggttaacttactcaaagaaccagtccagaaccttattgaagagatgagcccgcgaccaagctgtctaatctctgatatgtgtttgtcgtatacaagcgaaatcgccaagaagttcaaaataccaaagatcctcttccatggcatgggttgcttttgtcttctgtgtgttaacgttctgcgcaagaaccgtgagatcttggacaatttaaagtctgataaggagtacttcattgttccttattttcctgatagagttgaattcacaagacctcaagttccggtggaaacatatgttcctgcaggctggaaagagatcttggaggatatggtagaagcggataagacatcttatggtgttatagtcaactcatttcaagagctcgaacctgcgtatgccaaagacttcaaggaggcaaggtctggtaaagcatggaccattggacctgtttccttgtgcaacaaggtaggagtagacaaagcagagaggggaaacaaatcagatattgatcaagatgagtgccttgaatggctcgattctaaggaaccgggatctgtgctctacgtttgccttggaagtatttgtaatcttcctctgtctcagctccttgagctgggactaggcctagaggaatcccaaagacctttcatctgggtcataagaggttgggagaaatacaaagagttagttgagtggttctcggaaagcggctttgaagatagaatccaagatagaggacttctcatcaaaggatggtcccctcaaatgcttatcctttcacatccttctgttggagggttcttaacgcactgcggatggaactcgactcttgaggggataactgctggtctaccaatgcttacatggccactatttgcagaccaattctgcaacgagaaactggtcgtacaaatactaaaagtcggtgtaagtgccgaggttaaagaggtcatgaaatggggagaagaagagaagataggagtgttggtggataaagaaggagtgaagaaggcagtggaagaactaatgggtgagagtgatgatgcaaaagagagaagaagaagagccaaagagcttggagaatcagctcacaaggctgtggaagaaggaggctcctctcattctaatatcactttcttgctacaagacataatgcaactagcacagtccaataattgagtatatgtcatattttcaaaggaatttaaacattctatagtttttgttttctgtatttgtgaaatttaaaacagagtcttagtt

SEQ ID NO: 22 - protein, 495 aa, Arabidopsis thaliana
MAFEKNNEPFPLHFVLFPFMAQGHMIPMVDIARLLAQRGVLITIVTTPHNAARFKNVLNRTIESGLPINLVQVKFPYQEAGLQEGQENMDLLTTMEQITSFFKAVNLLKEPVQNLIEEMSPRPSCLISDMCLSYTSEIAKKFKIPKILFHGMGCFCLLCVNVLRKNREILDNLKSDKEYFIVPYFPDRVEFTRPQVPVETYVPAGWKEILEDMVEADKTSYGVIVNSFQELEPAYAKDFKEARSGKAWTIGPVSLCNKVGVDKAERGNKSDIDQDECLEWLDSKEPGSVLYVCLGSICNLPLSQLLELGLGLEESQRPFIWVIRGWEKYKELVEWFSESGFEDRIQDRGLLIKGWSPQMLILSHPSVGGFLTHCGWNSTLEGITAGLPMLTWPLFADQFCNEKLVVQILKVGVSAEVKEVMKWGEEEKIGVLVDKEGVKKAVEELMGESDDAKERRRRAKELGESAHKAVEEGGSSHSNITFLLQDIMQLAQSNN

SEQ ID NO: 23 - DNA, 1550 bp, Arabidopsis thaliana
atggtttccgaaacaaccaaatcttctccacttcactttgttctcttccctttcatggctcaaggccacatgattcccatggttgatattgcaaggctcttggctcagcgtggtgtgatcataacaattgtcacgacgcctcacaatgcagcgaggttcaagaatgtcctaaaccgtgccattgagtctggcttgcccatcaacttagtgcaagtcaagtttccatatctagaagctggtttgcaagaaggacaagagaatatcgattctcttgacacaatggagcggatgatacctttctttaaagcggttaactttctcgaagaaccagtccagaagctcattgaagagatgaaccctcgaccaagctgtctaatttctgatttttgtttgccttatacaagcaaaatcgccaagaagttcaatatcccaaagatcctcttccatggcatgggttgcttttgtcttctgtgtatgcatgttttacgcaagaaccgtgagatcttggacaatttaaagtcagataaggagcttttcactgttcctgattttcctgatagagttgaattcacaagaacgcaagttccggtagaaacatatgttccagctggagactggaaagatatctttgatggtatggtagaagcgaatgagacatcttatggtgtgatcgtcaactcatttcaagagctcgagcctgcttatgccaaagactacaaggaggtaaggtccggtaaagcatggaccattggacccgtttccttgtgcaacaaggtaggagccgacaaagcagagaggggaaacaaatcagacattgatcaagatgagtgccttaaatggctcgattctaagaaacatggctcggtgctttacgtttgtcttggaagtatctgtaatcttcctttgtctcaactcaaggagctgggactaggcctagaggaatcccaaagacctttcatttgggtcataagaggttgggagaagtacaaagagttagttgagtggttctcggaaagcggctttgaagatagaatccaagatagaggacttctcatcaaaggatggtcccctcaaatgcttatcctttcacatccatcagttggagggttcctaacacactgtggttggaactcgactcttgaggggataactgctggtctaccgctacttacatggccgctattcgcagaccaattctgcaatgagaaattggtcgttgaggtactaaaagccggtgtaagatccggggttgaacagcctatgaaatggggagaagaggagaaaataggagtgttggtggataaagaaggagtgaagaaggcagtggaagaattaatgggtgagagtgatgatgcaaaagagagaagaagaagagccaaagagcttggagattcagctcacaaggctgtggaagaaggaggctcttctcattctaacatctctttcttgctacaagacataatggaactggcagaacccaataattgagtatacgtcatctttttaaaggaatttaaaaattaaatagttttgttttctgtatttgtgaa

SEQ ID NO: 24 - protein, 495 aa, Arabidopsis thaliana
MVSETTKSSPLHFVLFPFMAQGHMIPMVDIARLLAQRGVIITIVTTPHNAARFKNVLNRAIESGLPINLVQVKFPYLEAGLQEGQENIDSLDTMERMIPFFKAVNFLEEPVQKLIEEMNPRPSCLISDFCLPYTSKIAKKFNIPKILFHGMGCFCLLCMHVLRKNREILDNLKSDKELFTVPDFPDRVEFTRTQVPVETYVPAGDWKDIFDGMVEANETSYGVIVNSFQELEPAYAKDYKEVRSGKAWTIGPVSLCNKVGADKAERGNKSDIDQDECLKWLDSKKHGSVLYVCLGSICNLPLSQLKELGLGLEESQRPFIWVIRGWEKYKELVEWFSESGFEDRIQDRGLLIKGWSPQMLILSHPSVGGFLTHCGWNSTLEGITAGLPLLTWPLFADQFCNEKLVVEVLKAGVRSGVEQPMKWGEEEKIGVLVDKEGVKKAVEELMGESDDAKERRRRAKELGDSAHKAVEEGGSSHSNISFLLQDIMELAEPNN